SEQUENCE LISTING (22 sequences)

SEQ ID NO: 1 - protein, 20 aa, Homo sapiens
PQQRPYGYDQIMPKKPAEDE

SEQ ID NO: 2 - protein, 9 aa, Homo sapiens
SLLMWITQC

SEQ ID NO: 3 - protein, 9 aa, Homo sapiens
LLMWITQCF

SEQ ID NO: 4 - protein, 13 aa, Homo sapiens
APRGPHGGAASGL

SEQ ID NO: 5 - protein, 9 aa, Homo sapiens
LEFYLAMPF

SEQ ID NO: 6 - protein, 9 aa, Homo sapiens
ARGPESRLL

SEQ ID NO: 7 - protein, 9 aa, Homo sapiens
EVDPASNTY

SEQ ID NO: 8 - protein, 10 aa, Homo sapiens
GVYDGREHTV

SEQ ID NO: 9 - protein, 9 aa, Homo sapiens
NYKRCFPVI

SEQ ID NO: 10 - protein, 10 aa, Homo sapiens
ALYVDSLFFL

SEQ ID NO: 11 - protein, 9 aa, Homo sapiens
SSYGQQSEK

SEQ ID NO: 12 - DNA, 42 bp, synthetic construct
  source 1..42: mol_type = other DNA
ggatccaccatggagtcgcgcggtcgccgttgtcccgaaatg

SEQ ID NO: 13 - protein, 5 aa, synthetic construct
GSSSS

SEQ ID NO: 14 - protein, 4 aa, synthetic construct
GSSS

SEQ ID NO: 15 - protein, 5 aa, synthetic construct
GPGPG

SEQ ID NO: 16 - protein, 5 aa, synthetic construct
EAAAK

SEQ ID NO: 17 - protein, 12 aa, synthetic construct
HEYGAEALERAG

SEQ ID NO: 18 - protein, 4 aa, synthetic construct
RVRR

SEQ ID NO: 19 - protein, 9 aa, Homo sapiens
YGYDQIMPK

SEQ ID NO: 20 - protein, 9 aa, Homo sapiens
RPYGYDQIM

SEQ ID NO: 21 - protein, 9 aa, Homo sapiens
GYDQIMPKK

SEQ ID NO: 22 - protein, 9 aa, Human immunodeficiency virus
AVDLSHFLK

(19) United States
(12) Patent Application Publication
(10) Pub. No.: US 2026/0306600 A1
(43) Pub. Date: Oct. 8, 2026

(54) SARCOMA CANCER VACCINES AND USES THEREOF

(71) Applicants: MEMORIAL SLOAN-KETTERING CANCER CENTER, New York, NY (US); MEMORIAL HOSPITAL FOR CANCER AND ALLIED DISEASES, New York, NY (US); SLOAN-KETTERING INSTITUTE FOR CANCER RESEARCH, New York, NY (US)

(72) Inventors: Christopher A. Klebanoff, New York, NY (US); Sandra D'Angelo, New York, NY (US)

(21) Appl. No.: 19/481,504

(22) PCT Filed: May 3, 2024
(86) PCT No.: PCT/US2024/027836

§ 371 Date: Nov. 4, 2025

Related U.S. Application Data
(60) Provisional application No. 63/500,375, filed on May 5, 2023.

(51) Int. Cl. A61K 39/00; A61K 39/395; A61P 37/04

(52) U.S. Cl. CPC A61K 39/001184; A61K 39/39558; A61P 37/04; A61K 2039/53; A61K 2039/80

(57) ABSTRACT

The present disclosure provides vaccine compositions comprising recombinant SS 18::SSX fusion peptide epitopes and at least one cancer-testis antigen (CTA) epitope, or nucleic acids (e.g., mRNA, cDNA) encoding the same, and methods for using the same to treat sarcoma (e.g., synovial sarcoma) in a subject in need thereof.

### CROSS-REFERENCE TO RELATED APPLICATIONS

This application is the U.S. national phase entry under 35 U.S.C. § 371 of International Application No. PCT/US2024/027836 filed May 3, 2024, which claims the benefit of and priority to U.S. Provisional Patent Application No. 63/500,375, filed May 5, 2023, the contents of which are incorporated herein by reference in their entireties.

### SEQUENCE LISTING

The contents of the .xml file submitted electronically herewith are incorporated herein by reference in their entirety, filename: 115872-3375_SL.xml; Size: 20,480 bytes; and Date of Creation: Nov. 3, 2025.

### TECHNICAL FIELD

The present technology relates generally to vaccine compositions comprising recombinant SS18::SSX fusion peptide epitopes and at least one cancer-testis antigen (CTA) epitope, or nucleic acids (e.g., mRNA, cDNA) encoding the same, and methods for using the same to treat sarcoma (e.g., synovial sarcoma) in a subject in need thereof.

### BACKGROUND

The following description of the background of the present technology is provided simply as an aid in understanding the present technology and is not admitted to describe or constitute prior art to the present technology.

Synovial sarcoma (SS) accounts for 10% to 20% of soft tissue sarcomas in adolescents and young adults. While localized disease can be cured through surgery with or without radiation and chemotherapy, patients with metastatic disease, which is present in up to 13% of patients at diagnosis and develops in 30-70% of patients with initially localized tumors, face a dismal prognosis. Response rates to standard chemotherapy range from 10-30%; tyrosine kinase inhibitors such as pazopanib offer response rates of about 10% and median progression-free survival of 4 months. Adoptive transfer of T cells genetically engineered to express TCRs targeting peptides derived from the cancer-testis antigens (CTAs) NY-ESO-1 and MAGE-A4 cause cancer regression in a subset of SS patients. Their limited efficacy results at least part from the non-universal or heterogeneous expression of these CTAs in SS. Because both TCRs are restricted by human leukocyte antigen (HLA)-A*02, these treatments are only applicable to ~30% of North American patients. Moreover, despite a relatively high overall response rate (ORR: 36%-61%), most patients experience disease relapse within months of treatment.

Therefore, there is an urgent need for more effective therapies for patients with metastatic SS. Moreover, there is an urgent need to develop novel immunotherapy approaches that simultaneously: 1) expand the number of SS patients eligible for TCR-based therapies by targeting peptides presented by prevalent HLA alleles besides HLA-A*02, and 2) increase the frequency and durability of responses.

### SUMMARY OF THE PRESENT TECHNOLOGY

In one aspect, the present disclosure provides a vaccine comprising a polypeptide comprising at least one SS18::SSX fusion junction epitope and an oligomer of at least one cancer-testis antigen (CTA) epitope, wherein the at least one SS18::SSX fusion junction sequence comprises the amino acid sequence of PQQRPYGYDQIMPKKPAEDE (SEQ ID NO: 1), optionally wherein the at least one SS18::SSX fusion junction epitope is operably linked to the oligomer. In another aspect, the present disclosure provides a vaccine comprising a nucleic acid molecule encoding a polypeptide comprising at least one SS18::SSX fusion junction epitope and an oligomer of at least one cancer-testis antigen (CTA) epitope, wherein the at least one SS18::SSX fusion junction sequence comprises the amino acid sequence of PQQRPYGYDQIMPKKPAEDE (SEQ ID NO: 1), optionally wherein the at least one SS18::SSX fusion junction epitope is operably linked to the oligomer. In some embodiments of the vaccine disclosed herein, the at least one SS18::SSX fusion junction epitope comprises the amino acid sequence of SEQ ID NO: 1 with 1, 2, 3, 4, 5, 6, 7, 8, 9, or 10 amino acid residues inserted at the N-terminus, the C-terminus, or both. In other embodiments of the vaccine disclosed herein, the at least one SS18::SSX fusion junction epitope comprises the amino acid sequence of SEQ ID NO: 1 with 1, 2, 3, 4, 5, 6, 7, or 8 amino acid residues deleted at the N-terminus, or the C-terminus. Additionally or alternatively, in some embodiments, the at least one SS18::SSX fusion junction epitope comprises an amino acid sequence that has about 90%, about 91%, about 92%, about 93%, about 94%, about 95%, about 96%, about 97%, about 98%, or about 99% identity to SEQ ID NO: 1.

Additionally or alternatively, in some embodiments, the nucleic acid molecule comprises RNA or DNA. The at least one SS18::SSX fusion junction epitope may be located at the N-terminus or the C-terminus of the oligomer. Additionally or alternatively, in certain embodiments, the polypeptide further comprises a leader peptide at its N-terminus and an MHC class I trafficking signal (MITD) at its C terminus. In some embodiments, the at least one SS18::SSX fusion junction epitope comprises exon 10 or exon 11 of SS18 and exon 5 or exon 11 of SSX1 or SSX2.

Additionally or alternatively, in some embodiments of the vaccine disclosed herein, the at least one CTA epitope is derived from one or more of NY-ESO-1, PRAME, or MAGEA4. In some embodiments, the at least one CTA epitope comprises a NY-ESO-1 epitope comprising one or more amino acid sequences selected from among SLLMWITQC (SEQ ID NO: 2), LLMWITQCF (SEQ ID NO: 3), APRGPHGGAASGL (SEQ ID NO: 4), LEFYLAMPF (SEQ ID NO: 5) and ARGPESRLL (SEQ ID NO: 6). In certain embodiments, the at least one CTA epitope comprises a MAGE-A4 epitope comprising one or more amino acid sequences selected from among EVDPASNTY (SEQ ID NO: 7), GVYDGREHTV (SEQ ID NO: 8), and NYKRCFPVI (SEQ ID NO: 9). Additionally or alternatively, in some embodiments, the at least one CTA epitope comprises a PRAME epitope comprising the amino acid sequence of ALYVDSLFFL (SEQ ID NO: 10).

Additionally or alternatively, in some embodiments, the oligomer of the at least one CTA epitope may include any one or more of the peptides comprising the amino acid sequence of any one or more of SEQ ID NOs: 2-10 with 1, 2, 3, 4, 5, 6, 7, 8, 9, or 10 amino acid residues inserted at the N-terminus, the C-terminus, or both. Additionally or alternatively, in certain embodiments, the oligomer of the at least one CTA epitope may include any one or more of the peptides comprising the amino acid sequence of any one or more of SEQ ID NOs: 2-10 with 1, 2, 3, 4, or 5 amino acid residues deleted at the N-terminus, or the C-terminus. In some embodiments, the oligomer of the at least one CTA epitope may include one or more peptides having about 90%, about 91%, about 92%, about 93%, about 94%, about 95%, about 96%, about 97%, about 98%, or about 99% identity to any one of the sequences of SEQ ID NOs: 2-10.

Additionally or alternatively, in certain embodiments of the vaccine disclosed herein, the oligomer is a dimer, a trimer, a tetramer, a pentamer, a hexamer, a heptamer, an octamer, a nonamer, a decamer, an 11-mer, a 12-mer, a 13-mer, a 14-mer, a 15-mer, a 16-mer, a 17-mer, an 18-mer, a 19-mer, a 20-mer, a 21-mer, a 22-mer, a 23-mer, a 24-mer, a 25-mer, a 26-mer, a 27-mer, a 28-mer, a 29-mer, or a 30-mer.

In any of the preceding embodiments of the vaccine disclosed herein, the oligomer of the at least one CTA epitope comprises tandem repeats of a single CTA epitope, optionally wherein the tandem repeats of the single CTA epitope are linked via a linker. In other embodiments, the oligomer of the at least one CTA epitope comprises at least 2 distinct CTA epitopes, optionally wherein the at least 2 distinct CTA epitopes are linked via a linker. The linker may be a cleavable peptide linker or a rigid peptide linker. Examples of linker include, but are not limited to, a GS4 linker (SEQ ID NO: 13), a GS3 linker (SEQ ID NO: 14), a P2A linker, a T2A linker, an E2A linker, a F2A linker, a BmCPV2A linker, an AAY linker, a GPGPG linker (SEQ ID NO: 15), an EAAAK linker (SEQ ID NO: 16), a HEYGAEALERAG linker (SEQ ID NO: 17), a KK linker, or an RVRR linker (SEQ ID NO: 18). Additionally or alternatively, in certain embodiments, the oligomer of the at least one CTA epitope further comprises one or more sequences of any one of SEQ ID NOs: 2-10.

In any and all embodiments of the vaccine disclosed herein, the nucleic acid molecule comprises RNA, and wherein each uracil residue in the RNA comprises a chemical modification and/or at least one 5′ terminal cap. In some embodiments, the chemical modification is in the 5-position of each uracil residue. In certain embodiments, the chemical modification is selected from the group consisting of pseudouridine, N1-methylpseudouridine, 2-thiouridine, 4′-thiouridine, 5-methylcytosine, 2-thio-1-methyl-1-deaza-pseudouridine, 2-thio-l-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine, and 2′-O-methyl uridine. Additionally or alternatively, in some embodiments, the at least one 5′ terminal cap is a naturally occurring cap, a synthetic cap or an optimized cap.

Additionally or alternatively, in some embodiments, the vaccine further comprises a pharmaceutically acceptable carrier selected from the group consisting of a cream, emulsion, gel, liposome, nanoparticle, or ointment. In certain embodiments, the vaccine is formulated within a nanoparticle, wherein the nanoparticle has a mean diameter of 50-200 nm. In further embodiments, the nanoparticle is a lipid nanoparticle. In certain embodiments, the nanoparticle has a polydiversity value of less than 0.4 or a net neutral charge at a neutral pH.

In any of the above embodiments of the vaccine disclosed herein, the nanoparticle is a cationic lipid nanoparticle comprising a cationic lipid, a PEG-modified lipid, a sterol and a non-cationic lipid. The cationic lipid may be selected from the group consisting of 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319). In some embodiments, the cationic lipid nanoparticle has a molar ratio of about 20-60% cationic lipid, about 5-25% non-cationic lipid, about 25-55% sterol; and about 0.5-15% PEG-modified lipid.

In any and all embodiments of the vaccine disclosed herein, the SS18::SSX fusion junction epitope and/or the at least one CTA epitope are presented by one or more of HLA-A*02:01, HLA-A*03:01, HLA-A*11:01, HLA-B*07:02, HLA-C*04:01, HLA-C*12:03, HLA-A *01:01, HLA-A *24:02, HLA-A *24:10, HLA-A *01:02, HLA-A *68:01, HLA-A *26:01, HLA-B*57:02, HLA-B*14:02, HLA-B* 35:01, HLA-B*38:01, HLA-B*41:01, HLA-B*49:01, HLA-B*39:06, HLA-B* 18:02, HLA-B*40:02, HLA-B*35:02, HLA-B*51:01, HLA-B*57:01, HLA-B*18:01, HLA-B*44:05, HLA-C*17:01, HLA-C*15:04, HLA-C*06:02, HLA-C*07:01, HLA-C* 02:02, HLA-C*03:04, HLA-C*07:02, HLA-C*08:02, or HLA-C*07:02.

In one aspect, the present disclosure provides a method for preventing or treating sarcoma in a patient in need thereof comprising administering to the patient an effective amount of any and all embodiments of the vaccine described herein. The sarcoma may be a soft tissue sarcoma, a synovial sarcoma, a myxoid-round cell liposarcoma, a malignant peripheral nerve sheath tumor, or a undifferentiated pleiomorphic sarcoma. In some embodiments, the sarcoma is recurrent or metastatic. In certain embodiments, the patient has received at least one prior anti-cancer therapy, optionally wherein the at least one prior anti-cancer therapy is surgery, radiotherapy, or chemotherapy.

Additionally or alternatively, in some embodiments, the method further comprises sequentially, simultaneously or separately administering to the subject an effective amount of an immune checkpoint inhibitor. In certain embodiments, the immune checkpoint inhibitor comprises one or more of an anti-PD-1 antibody, an anti-PD-L1 antibody, an anti-PD-L2 antibody, an anti-CTLA-4 antibody, an anti-TIM3 antibody, an anti-4-1BB antibody, an anti-CD73 antibody, an anti-GITR antibody, and an anti-LAG-3 antibody. Examples of anti-PD-L1 antibodies include envafolimab, atezolizumab, avelumab, or durvalumab. Examples of anti-PD-1 antibodies include pembrolizumab, nivolumab, or cemiplimab. In certain embodiments, the CTLA-4 antibody comprises ipilimumab or tremelimumab.

In any and all embodiments of the methods disclosed herein, the patient harbors a loss-of-function in SETD2. In some embodiments, the vaccine and/or the immune checkpoint inhibitor is administered pleurally, topically, parenterally, intramuscularly, intravenously, subcutaneously, intranodally, intratumorally, intrathecally, intrapleurally or intraperitoneally.

### BRIEF DESCRIPTION OF THE DRAWINGS

FIG. 1: Example of copy number loss at the SETD2 locus (arrow) in a synovial sarcoma (SS) patient in the absence of widespread copy number changes.

FIG. 2: SETD2 loss of function in SS is detectable by IHC and correlates with complete loss of its catalytic product, H3K36me3, while H3K36mez2 is preserved.

FIG. 3: H3K36me3 is lost from chromatin in a SETD2^{-/-} (vs. WT) HS-SY-II human SS cell line expressing FLAG-HA-SS18::SSX. Metaplot shoring normalized H3K36me3 signal over gene bodies.

FIGS. 4A-4C: SS is characterized by a pathognomic t(X;18) chromosomal translocation that generates recurrent SS18::SSX1 and $S18::SSX2 fusion proteins containing a highly conserved junctional amino acid sequence. FIG. 4A: Schematic of the protein sequences for wildtype (WT) SS18, WT SSX1/2, and the resulting chimeric proteins. FIG. 4B: Conserved junctional amino acid sequences. Figure discloses SEQ ID NOs: 1 and 1, respectively in order of appearance. FIG. 4C: Predicted (NetMHC 4.0) and experimentally validated fusion-derived peptide binders to prevalent HLA-I alleles. Figure discloses SEQ ID NOs: 19, 19, 20, 21, and 19, respectively, in order of appearance.

FIGS. 5A-5F: An HLA-immunoprecipitation/mass spectrometry (HLA-IP/MS) screen identified two immunogenic “public” neoantigens resulting from the EWSR1-WT1 fusion protein. FIG. 5A: Overview of an HLA-IP/MS screen including the most common (≥10%) HLA-A, -B, and -C alleles among North American patients. FIG. 5B: Discovered public neoantigens derived from the EWSR1-WT1 exon 7/8 fusion protein. Figure discloses SEQ ID NOs: 11 and 11, respectively in order of appearance. FIG. 5C: Validation of peptide sequence by overlay of experimental and synthetic MS spectra. FIGS. 5D-5E: Establishment of a peripheral blood mononuclear cell (PBMC) biobank from Desmoplastic small round cell tumor (DSCRT) patients whose cancer expresses EWSR1=WT17/s (fusion⁺) and either HLA-A*03 or HLA-A*11 (HLA-I⁺) using MSK-IMPACT. FIG. 5F: FACS plot from PBMCs of a fusion⁺/HLA-A*11′ DSRCT patient using dual color HLA-A*11 dextramers (Dex) loaded with the fusion-derived peptide. Figure discloses SEQ ID NOs: 11 and 22, respectively, in order of appearance.

FIG. 6: Repository of HLA-typed, SS18::SSX(1/2)⁺ SS patient derived xenografts (PDXs).

FIGS. 7A-7B: SSmRNA vaccine design. FIG. 7A: Schematic of the concatenated SSmRNA vaccine. The vaccine contains n=10 immunogenic epitopes derived from NY-ESO-1, MAGE-A4, and PRAME in addition to the conserved junctional amino sequence resulting from the SS18-SSX(1/2) fusion proteins. FIG. 7B: Empirically validated epitopes resulting from CTAs commonly expressed by SS (top) and the shared junctional amino acid sequence resulting from the SS18::SSX(1/2) chimeric proteins (bottom). All epitopes listed are restricted by prevalent (>10%) HLA-I alleles. UTR, untranslated region; Poly-A, polyadenylation. Figure discloses SEQ ID NOs: 2-10 and 1, respectively, in order of appearance.

FIGS. 8A-8C: Overview of SSmRNA vaccine schedule and immune monitoring. Schedule of SSmRNA vaccine prime/boost administration (FIG. 8A) and immune monitoring (FIG. 8B). FIG. 8C: Immune monitoring studies to assess and quantify a vaccine-induced CD8+ and CD4+ T cell response.

FIG. 9 shows exemplary nucleic acid modifications.

FIG. 10 shows exemplary additional nucleic acid modifications.

### DETAILED DESCRIPTION

It is to be appreciated that certain aspects, modes, embodiments, variations and features of the present methods are described below in various levels of detail in order to provide a substantial understanding of the present technology. It is to be understood that the present disclosure is not limited to particular uses, methods, reagents, compounds, compositions or biological systems, which can, of course, vary. It is also to be understood that the terminology used herein is for the purpose of describing particular embodiments only, and is not intended to be limiting.

In practicing the present methods, many conventional techniques in molecular biology, protein biochemistry, cell biology, immunology, microbiology and recombinant DNA are used. See, e.g., Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology. Methods to detect and measure levels of polypeptide gene expression products (i.e., gene translation level) are well-known in the art and include the use of polypeptide detection methods such as antibody detection and quantification techniques. (See also, Strachan & Read, Human Molecular Genetics, Second Edition. (John Wiley and Sons, Inc., NY, 1999)).

Synovial sarcoma (SS) is driven by SS18::SSX-dependent chromatin dysregulation, suggesting that elucidating key aspects of this deregulation might point to therapeutic epigenetic vulnerabilities. At the same time, driver fusions such as SS18::SSX may generate a unique class of shared, or public, neoantigens (NeoAgs) because they create novel peptide sequences that diverge significantly from self-proteins. Specifically, the junctional amino acid sequence of the chimeric SS18::SSX proteins is unique to SS cells. In addition, there is evidence that epigenetic modulation may potentiate immunotherapy approaches.

Cancer vaccines often target multiple antigens to increase their immunogenicity. Investigators first reported prominent expression of CTAs, including NY-ESO-1 and MAGEA4 family, in SS over 20 years ago. The CTAs are each expressed in approximately 80% of SS. Without wishing to be bound by theory, it is hypothesized that co-targeting of the SS18::SSX fusion and SS-expressed CTAs should yield strong, effective antitumor immune responses. The present disclosure identifies epigenetic and immuno-oncologic vulnerabilities in SS and potential synergies between them towards long-term responses in metastatic SS.

### Definitions

Before describing the disclosed embodiments in detail, it is to be understood that the present disclosure is not limited to particular compositions or biological systems, which can, of course, vary. It is also to be understood that the terminology used herein is for the purpose of describing particular embodiments only and is not intended to be limiting.

Unless defined otherwise, all technical and scientific terms used herein generally have the same meaning as commonly understood by one of ordinary skill in the art to which this technology belongs. As used in this specification and the appended claims, the singular forms “a”, “an” and “the” include plural referents unless the content clearly dictates otherwise. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art. It is understood that embodiments of the present application described herein include “consisting of” and/or “consisting essentially of” embodiments.

As used herein, reference to “not” a value or parameter generally means and describes “other than” a value or parameter. For example, the method is not used to treat cancer of type X means the method is used to treat cancer of types other than X.

As used herein, the term “about” in reference to a number is generally taken to include numbers that fall within a range of 1%, 5%, or 10% in either direction (greater than or less than) of the number unless otherwise stated or otherwise evident from the context (except where such number would be less than 0% or exceed 100% of a possible value).

As used herein, the “administration” of an agent or drug to a subject includes any route of introducing or delivering to a subject a compound to perform its intended function. Administration can be carried out by any suitable route, including but not limited to, orally, intranasally, parenterally (intravenously, intramuscularly, intraperitoneally, or subcutaneously), rectally, intrathecally, intratumorally or topically. Administration includes self-administration and the administration by another. “Administration” of a cell or vector or other agent and compositions containing same can be performed in one dose, continuously or intermittently throughout the course of treatment. Methods of determining the most effective means and dosage of administration are known to those of skill in the art and will vary with the composition used for therapy, the purpose of the therapy, the target cell being treated, and the subject being treated. Single or multiple administrations can be carried out with the dose level and pattern being selected by the treating physician or in the case of animals, by the treating veterinarian. In some embodiments, administering or a grammatical variation thereof also refers to more than one doses with certain interval. In some embodiments, the interval is 1 day, 2 days, 3 days, 4 days, 5 days, 6 days, 1 week, 10 days, 2 weeks, 3 weeks, 1 month, 2 months, 3 months, 4 months, 5 months, 6 months, 1 year or longer. In some embodiments, one dose is repeated for once, twice, three times, four times, five times, six times, seven times, eight times, nine times, ten times or more. Suitable dosage formulations and methods of administering the agents are known in the art. Route of administration can also be determined and method of determining the most effective route of administration are known to those of skill in the art and will vary with the composition used for treatment, the purpose of the treatment, the health condition or disease stage of the subject being treated, and target cell or tissue. Non-limiting examples of route of administration include oral administration, intraperitoneal, infusion, nasal administration, inhalation, injection, and topical application. In some embodiments, the administration is an infusion (for example to peripheral blood of a subject) over a certain period of time, such as about 30 minutes, about 1 hour, about 2 hours, about 3 hours, about 4 hours, about 5 hours, about 6 hours, about 7 hours, about 8 hours, about 9 hours, about 10 hours, about 11 hours, about 12 hours, about 24 hours or longer.

An “adjuvant” refers to one or more substances that cause stimulation of the immune system. In this context, an adjuvant is used to enhance an immune response to one or more vaccine antigens or antibodies. An adjuvant may be administered to a subject before, in combination with, or after administration of the vaccine. Examples of chemical compounds used as adjuvants include aluminum compounds, oils, block polymers, immune stimulating complexes, vitamins and minerals (e.g., vitamin E, vitamin A, selenium, and vitamin B12), Quil A (saponins), bacterial and fungal cell wall components (e.g., lipopolysaccharides, lipoproteins, and glycoproteins), hormones, cytokines, and costimulatory factors.

The term “amino acid” refers to naturally occurring and non-naturally occurring amino acids, as well as amino acid analogs and amino acid mimetics that function in a manner similar to the naturally occurring amino acids. Naturally encoded amino acids are the 20 common amino acids (alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine) and pyrolysine and selenocysteine. Amino acid analogs refer to agents that have the same basic chemical structure as a naturally occurring amino acid, i.e., an a carbon that is bound to a hydrogen, a carboxyl group, an amino group, and an R group, such as, homoserine, norleucine, methionine sulfoxide, methionine methyl sulfonium. Such analogs have modified R groups (such as, norleucine) or modified peptide backbones, but retain the same basic chemical structure as a naturally occurring amino acid. In some embodiments, amino acids forming a polypeptide are in the D form. In some embodiments, the amino acids forming a polypeptide are in the L form. In some embodiments, a first plurality of amino acids forming a polypeptide are in the D form, and a second plurality of amino acids are in the L form.

Amino acids are referred to herein by either their commonly known three letter symbols or by the one-letter symbols recommended by the IUPAC-IUB Biochemical Nomenclature Commission. Nucleotides, likewise, are referred to by their commonly accepted single-letter code.

The terms “cancer” or “tumor” are used interchangeably and refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells can exist alone within an animal, or can be a non-tumorigenic cancer cell. As used herein, the term “cancer” includes premalignant, as well as malignant cancers.

As used herein, “complementary” sequences refer to two nucleotide sequences which, when aligned anti-parallel to each other, contain multiple individual nucleotide bases which pair with each other. Paring of nucleotide bases forms hydrogen bonds and thus stabilizes the double strand structure formed by the complementary sequences. It is not necessary for every nucleotide base in two sequences to pair with each other for sequences to be considered “complementary”. Sequences may be considered complementary, for example, if at least 30%, 40%, 50%, 55%, 60%, 65%, 70%, 75%, 80%, 85%, 90%, 95%, 98%, 99%, or 100% of the nucleotide bases in two sequences pair with each other. In some embodiments, the term complementary refers to 100% of the nucleotide bases in two sequences pair with each other. In addition, sequences may still be considered “complementary” when the total lengths of the two sequences are significantly different from each other. For example, a primer of 15 nucleotides may be considered “complementary” to a longer polynucleotide containing hundreds of nucleotides if multiple individual nucleotide bases of the primer pair with nucleotide bases in the longer polynucleotide when the primer is aligned anti-parallel to a particular region of the longer polynucleotide. Nucleotide bases paring is known in the field, such as in DNA, the purine adenine (A) pairs with the pyrimidine thymine (T) and the pyrimidine cytosine (C) always pairs with the purine guanine (G); while in RNA, adenine (A) pairs with uracil (U) and guanine (G) pairs with cytosine (C). Further, the nucleotide bases aligned anti-parallel to each other in two complementary sequences, but not a pair, are referred to herein as a mismatch.

As used herein, a “control” is an alternative sample used in an experiment for comparison purpose. A control can be “positive” or “negative.” For example, where the purpose of the experiment is to determine a correlation of the efficacy of a therapeutic agent for the treatment for a particular type of disease, a positive control (a compound or composition known to exhibit the desired therapeutic effect) and a negative control (a subject or a sample that does not receive the therapy or receives a placebo) are typically employed.

As used herein, the term “effective amount” refers to a quantity sufficient to achieve a desired therapeutic and/or prophylactic effect, e.g., an amount which results in the prevention of, or a decrease in a disease or condition described herein or one or more signs or symptoms associated with a disease or condition described herein. In the context of therapeutic or prophylactic applications, the amount of a composition administered to the subject will vary depending on the composition, the degree, type, and severity of the disease and on the characteristics of the individual, such as general health, age, sex, body weight and tolerance to drugs. The skilled artisan will be able to determine appropriate dosages depending on these and other factors. The compositions can also be administered in combination with one or more additional therapeutic compounds. In the methods described herein, the therapeutic compositions may be administered to a subject having one or more signs or symptoms of a disease or condition described herein. As used herein, a “therapeutically effective amount” of a composition refers to composition levels in which the physiological effects of a disease or condition are ameliorated or eliminated. A therapeutically effective amount can be given in one or more administrations.

As used herein, “expression” includes one or more of the following: transcription of the gene into precursor mRNA; splicing and other processing of the precursor mRNA to produce mature mRNA; mRNA stability; translation of the mature mRNA into protein (including codon usage and tRNA availability); and glycosylation and/or other modifications of the translation product, if required for proper expression and function.

As used herein, an “expression vector” includes vectors capable of expressing DNA that is operably linked with regulatory sequences, such as promoter regions, that are capable of effecting expression of such DNA fragments. Such additional segments can include promoter and terminator sequences, and optionally can include one or more origins of replication, one or more selectable markers, an enhancer, a polyadenylation signal, and the like. Expression vectors are generally derived from plasmid or viral DNA, or can contain elements of both. Thus, an expression vector refers to a recombinant DNA or RNA construct, such as a plasmid, a phage, recombinant virus or other vector that, upon introduction into an appropriate host cell, results in expression of the cloned DNA. Appropriate expression vectors are well known to those of skill in the art and include those that are replicable in eukaryotic cells and/or prokaryotic cells and those that remain episomal or those which integrate into the host cell genome.

As used herein, the term “gene” means a segment of DNA that contains all the information for the regulated biosynthesis of an RNA product, including promoters, exons, introns, and other untranslated regions that control expression. Specifically, a gene refers to a DNA sequence that comprises regulatory and coding sequences necessary for the production of an RNA, which may have a non-coding function (e.g., a ribosomal or transfer RNA) or which may include a polypeptide or a polypeptide precursor. The RNA or polypeptide may be encoded by a full length coding sequence or by any portion of the coding sequence so long as the desired activity or function is retained. Although a sequence of the nucleic acids may be shown in the form of DNA, a person of ordinary skill in the art recognizes that the corresponding RNA sequence will have a similar sequence with the thymine being replaced by uracil, i.e., “T” is replaced with “U.”

As used herein, the term “heterologous nucleic acid molecule or polypeptide” refers to a nucleic acid molecule (e.g., a cDNA, DNA or RNA molecule) or polypeptide that is either not normally expressed or is expressed at an aberrant level in a cell or sample obtained from a cell. This nucleic acid can be from another organism, or it can be, for example, an mRNA molecule that is not normally expressed in a cell or sample.

As used herein, a “host cell” is a cell that is used to receive, maintain, reproduce and amplify an expression vector. A host cell also can be used to express the polypeptide encoded by the expression vector. The nucleic acid contained in the expression vector is replicated when the host cell divides, thereby amplifying the nucleic acids.

“Homology” or “identity” or “percent identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. A polynucleotide or polynucleotide region (or a polypeptide or polypeptide region) has a certain percentage (for example, at least 60%, 65%, 70%, 75%, 80%, 85%, 90%, 95%, 98% or 99%) of “sequence identity” to another sequence means that, when aligned, that percentage of bases (or amino acids) are the same in comparing the two sequences. This alignment and the percent homology or sequence identity can be determined using software programs known in the art. In some embodiments, default parameters are used for alignment. One alignment program is BLAST, using default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the National Center for Biotechnology Information. The percent homology between two amino acid sequences can be also determined using the algorithm of E. Meyers and W. Miller (Comput. Appl. Biosci., 4: 1 1-17 (1988)) which has been incorporated into the ALIGN program (version 2.0), using a PAM120 weight residue table, a gap length penalty of 12 and a gap penalty of 4. In addition, the percent homology between two amino acid sequences can be determined using the Needleman and Wunsch (J. Mol. Biol. 48:444-453 (1970)) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6. Biologically equivalent polynucleotides are those having the specified percent homology and encoding a polypeptide having the same or similar biological activity. Two sequences are deemed “unrelated” or “non-homologous” if they share less than 40% identity, or less than 25% identity, with each other.

As used herein, “immune checkpoint inhibitor” or “immune checkpoint blocking agent” or “immune checkpoint blockade inhibitor” refers to molecules that completely or partially reduce, inhibit, interfere with, or modulate the activity of one or more checkpoint proteins. Checkpoint proteins regulate T-cell activation or function. Checkpoint proteins include, but are not limited to, CD28 receptor family members, CTLA-4 and its ligands CD80 and CD86; PD-1 and its ligands PD-L1 and PD-L2; LAG3, B7-H3, B7-H4, TIM3, ICOS, II DLBCL, BTLA or any combination of two or more of the foregoing. Non-limiting examples of immune checkpoint blocking agents contemplated for use herein include, but are not limited to, inhibitory antibodies against CD28 inhibitor such as CTLA-4 (cytotoxic T lymphocyte antigen 4) (e.g., ipilimumab), anti-PD-1 (programmed Death 1) inhibitory antibodies (e.g., nivolumab, pembrolizumab, pidilizumab, lambrolizumab), and anti-PD-L1 (Programmed death ligand 1) inhibitory antibodies (MPDL3280A, BMS-936559, MEDI4736, MSB 00107180), as well as inhibitory antibodies against LAG-3 (lymphocyte activation gene 3), TIM3 (T-cell immunoglobulin and mucin-3), B7-H3, TIGIT (T-cell immunoreceptor with Ig and ITIM domains), AMP-224, MDX-1105, arelumab, tremelimumab, IMP321, MGA271, BMS-986016, lirilumab, urelumab, PF-05082566, IPH2101, MEDI-6469, CP-870,893, Mogamulizumab, Varlilumab, Galiximab, AMP-514, AUNP 12, Indoximod, NLG-919, INCB024360, CD80, CD86, ICOS, DLBCL inhibitors, or BTLA, PDR001, and combinations thereof.

As used herein, “immune response” refers to the action of one or more of lymphocytes, antigen presenting cells, phagocytic cells, granulocytes, and soluble macromolecules produced by the above cells or the liver (including antibodies, cytokines, and complement) that results in selective damage to, destruction of, or elimination from the human body of cancerous cells, metastatic tumor cells, etc. An immune response may include a cellular response, such as a T-cell response that is an alteration (modulation, e.g., significant enhancement, stimulation, activation, impairment, or inhibition) of cellular, i.e., T-cell function. A T-cell response may include generation, proliferation or expansion, or stimulation of a particular type of T-cell, or subset of T-cells, for example, effector CD4⁺, CD4⁺ helper, effector CD8⁺, CD8⁺ cytotoxic, or natural killer (NK) cells. Such T-cell subsets may be identified by detecting one or more cell receptors or cell surface molecules (e.g., CD or cluster of differentiation molecules). A T-cell response may also include altered expression (statistically significant increase or decrease) of a cellular factor, such as a soluble mediator (e.g., a cytokine, lymphokine, cytokine binding protein, or interleukin) that influences the differentiation or proliferation of other cells. For example, Type I interferon (IFN-α/β) is a critical regulator of the innate immunity (Huber et al., Immunology 132(4):466-474 (2011)). Animal and human studies have shown a role for IFN-α/β in directly influencing the fate of both CD4⁺ and CD8⁺ T-cells during the initial phases of antigen recognition and anti-tumor immune response. IFN Type I is induced in response to activation of dendritic cells, in turn a sentinel of the innate immune system. An immune response may also include humoral (antibody) response.

As used herein, “operably linked” with reference to nucleic acid sequences, regions, elements or domains means that the nucleic acid regions are functionally related to each other. For example, a nucleic acid encoding a leader peptide can be operably linked to a nucleic acid encoding a polypeptide, whereby the nucleic acids can be transcribed and translated to express a functional fusion protein, wherein the leader peptide affects secretion of the fusion polypeptide. In some instances, the nucleic acid encoding a first polypeptide (e.g., a leader peptide) is operably linked to nucleic acid encoding a second polypeptide and the nucleic acids are transcribed as a single mRNA transcript, but translation of the mRNA transcript can result in one of two polypeptides being expressed. For example, an amber stop codon can be located between the nucleic acid encoding the first polypeptide and the nucleic acid encoding the second polypeptide, such that, when introduced into a partial amber suppressor cell, the resulting single mRNA transcript can be translated to produce either a fusion protein containing the first and second polypeptides, or can be translated to produce only the first polypeptide. In another example, a promoter can be operably linked to nucleic acid encoding a polypeptide, whereby the promoter regulates or mediates the transcription of the nucleic acid.

As used herein, by “pharmaceutically acceptable” or “pharmacologically compatible” is meant a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S. Food and Drug administration.

The terms “polynucleotide”, “nucleic acid” and “oligonucleotide” are used interchangeably and refer to a polymeric form of nucleotides of any length, either deoxyribonucleotides or ribonucleotides or analogs thereof, in modified or unmodified form. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. Unless otherwise specified or required, any embodiment of this disclosure that is a polynucleotide encompasses both the double-stranded form and each of two complementary single-stranded forms known or predicted to make up the double-stranded form. A polynucleotide is composed of a specific sequence of four nucleotide bases: adenine (A); cytosine (C); guanine (G); thymine (T); and uracil (U) for thymine when the polynucleotide is RNA. Thus, the term “polynucleotide sequence” is the alphabetical representation of a polynucleotide molecule. This alphabetical representation can be input into databases in a computer having a central processing unit and used for bioinformatics applications such as functional genomics and homology searching. Polynucleotides include, without limitation, single- and double-stranded DNA, DNA that is a mixture of single- and double-stranded regions, single- and double-stranded RNA, RNA that is mixture of single- and double-stranded regions, and hybrid molecules comprising DNA and RNA that may be single-stranded or, more typically, double-stranded or a mixture of single- and double-stranded regions. In addition, polynucleotide refers to triple-stranded regions comprising RNA or DNA or both RNA and DNA. The term polynucleotide also includes DNAs or RNAs containing one or more modified bases and DNAs or RNAs with backbones modified for stability or for other reasons.

The terms “polypeptide,” “peptide,” and “protein” are used interchangeably herein to refer to a polymer of amino acid residues. The terms apply to naturally occurring amino acid polymers as well as amino acid polymers in which one or more amino acid residues are a non-naturally occurring amino acid, e.g., an amino acid analog. The terms encompass amino acid chains of any length, including full length proteins, wherein the amino acid residues are linked by covalent peptide bonds.

As used herein, “prevention” or “preventing” of a disorder or condition refers to a compound that, in a statistical sample, reduces the occurrence of the disorder or condition in the treated sample relative to an untreated control sample, or delays the onset of one or more symptoms of the disorder or condition relative to the untreated control sample.

As used herein, the term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the material is derived from a cell so modified. Thus, for example, recombinant cells express genes that are not found within the native (non-recombinant) form of the cell or express native genes that are otherwise abnormally expressed, under expressed or not expressed at all.

As used herein, a “sample” refers to a substance that is being assayed for the presence of a mutation or alteration in a nucleic acid of interest. Processing methods to release or otherwise make available a nucleic acid for detection are well known in the art and may include steps of nucleic acid manipulation. A biological sample may be a body fluid or a tissue sample. In some cases, a biological sample may consist of or comprise blood, plasma, sera, urine, feces, epidermal sample, vaginal sample, skin sample, cheek swab, sperm, amniotic fluid, cultured cells, bone marrow sample, tumor biopsies, aspirate and/or chorionic villi, cultured cells, and the like. Fresh, fixed or frozen tissues may also be used. In one embodiment, the sample is preserved as a frozen sample or as formaldehyde- or paraformaldehyde-fixed paraffin-embedded (FFPE) tissue preparation. For example, the sample can be embedded in a matrix, e.g., an FFPE block or a frozen sample. Whole blood samples of about 0.5 to 5 ml collected with EDTA, ACD or heparin as anti-coagulant are suitable.

As used herein, the term “separate” therapeutic use refers to an administration of at least two active ingredients at the same time or at substantially the same time by different routes.

As used herein, the term “sequential” therapeutic use refers to administration of at least two active ingredients at different times, the administration route being identical or different. More particularly, sequential use refers to the whole administration of one of the active ingredients before administration of the other or others commences. It is thus possible to administer one of the active ingredients over several minutes, hours, or days before administering the other active ingredient or ingredients. There is no simultaneous treatment in this case.

As used herein, the term “simultaneous” therapeutic use refers to the administration of at least two active ingredients by the same route and at the same time or at substantially the same time.

As used herein, the terms “subject”, “patient”, or “individual” can be an individual organism, a vertebrate, a mammal, or a human. In some embodiments, the subject, patient or individual is a human.

“Treating” or “treatment” as used herein covers the treatment of a disease or disorder described herein, in a subject, such as a human, and includes: (i) inhibiting a disease or disorder, i.e., arresting its development; (ii) relieving a disease or disorder, i.e., causing regression of the disorder; (iii) slowing progression of the disorder; and/or (iv) inhibiting, relieving, or slowing progression of one or more symptoms of the disease or disorder. Therapeutic effects of treatment include, without limitation, inhibiting recurrence of disease, alleviation of symptoms, diminishment of any direct or indirect pathological consequences of the disease, preventing metastases, decreasing the rate of disease progression, amelioration or palliation of the disease state, and remission or improved prognosis. By “treating a cancer” is meant that the symptoms associated with the cancer are, e.g., alleviated, reduced, cured, or placed in a state of remission.

It is also to be appreciated that the various modes of treatment of diseases as described herein are intended to mean “substantial,” which includes total but also less than total treatment, and wherein some biologically or medically relevant result is achieved. The treatment may be a continuous prolonged treatment for a chronic disease or a single, or few time administrations for the treatment of an acute condition.

As used herein, “tumor immunity” refers to one or more processes by which tumors evade recognition and clearance by the immune system. Thus, as a therapeutic concept, tumor immunity is “treated” when such evasion is attenuated or eliminated, and the tumors are recognized and attacked by the immune system (the latter being termed herein “anti-tumor immunity”). An example of tumor recognition is tumor binding, and examples of tumor attack are tumor reduction (in number, size, or both) and tumor clearance.

### SS18::SSX Fusion Presents Epigenetic and Immune Vulnerabilities

The defining genetic event of SS is the translocation of the SS18 gene on chromosome 18q11 to SSX1 or SSX2 on chromosome Xp11.2. Expression of SS18::SSX is both necessary and sufficient for sarcomagenesis, establishing the fusion as the primary oncogenic driver in this sarcoma. The resulting SS18::SSX fusion protein retains the transcriptional activation domain of SS18 and the transcriptional repressor domain of SSX. The SS18::SSX fusion incorporates into the cBAF complex, mediating epigenetic changes. The resulting epigenomic alterations induce targetable dependencies on specific epigenetic regulators.

In >90% of cases, the genomic translocation breakpoints involve the intronic regions between SS18 exons 10 and 11 and exons 5 and 6 of the highly homologous SSX1 or SSX2 genes. The amino acid sequences of SSX1 and SSX2 immediately adjacent to the fusion point are identical, simplifying the use of the fusion junction as an immunotherapy target. Consequently, the resulting junctional region of the chimeric SS18::SSX(1/2) proteins is shared by the majority of SS patients and is unique to cancer cells. This defining feature of SS raises the possibility of an “off-the-shelf” T cell receptor (TCR) immunotherapy targeting this shared neoantigen derived from the SS18::SSX protein. Because the fusion protein is required for sarcoma cell fitness, its expression Is conserved across metastases and in recurrent or progressive disease.

SS18::SSX1 physically interacts with PRC1.1 and co-associates with SWI/SNF and KDM2B complexes on unmethylated CpG islands to activate expression of PRC-regulated genes controlling mesenchymal differentiation. The CRISPR/Cas9 screen of genes involved in epigenetic regulation that implicated KDM2B as a vulnerability in SS also identified other members of PRC1.1 (such as BCOR and PCGF1) and of PRC1.3 (PCGF3) as required for SS18::SSX-driven transformation. The PCGF1- and PCGF3-containing complexes are distinct. While both complexes are responsible for depositing the H2Aub repressive mark, they may occupy and regulate different target genes.

SETD2 H3K36 methyltransferase gene is the most commonly altered epigenetic regulatory gene in SS. Its positive selection suggests cooperativity with SS18::SSX and nominates H3K36 as a critical site of epigenetic dysregulation in SS.

### Vaccines of the Present Technology

In one aspect, the present disclosure provides a vaccine comprising a polypeptide comprising at least one SS18::SSX fusion junction epitope and an oligomer of at least one cancer-testis antigen (CTA) epitope, wherein the at least one SS18::SSX fusion junction sequence comprises the amino acid sequence of PQQRPYGYDQIMPKKPAEDE (SEQ ID NO: 1), optionally wherein the at least one SS18::SSX fusion junction epitope is operably linked to the oligomer. In another aspect, the present disclosure provides a vaccine comprising a nucleic acid molecule encoding a polypeptide comprising at least one SS18::SSX fusion junction epitope and an oligomer of at least one cancer-testis antigen (CTA) epitope, wherein the at least one SS18::SSX fusion junction sequence comprises the amino acid sequence of PQQRPYGYDQIMPKKPAEDE (SEQ ID NO: 1), optionally wherein the at least one SS18::SSX fusion junction epitope is operably linked to the oligomer. In some embodiments, the nucleic acid molecule comprises RNA or DNA. The at least one SS18::SSX fusion junction epitope may be located at the N-terminus or the C-terminus of the oligomer. In some embodiments, the at least one SS18::SSX fusion junction epitope comprises exon 10 or exon 11 of SS18 and exon 5 or exon 11 of SSX1 or SSX2.

In some embodiments of the vaccine disclosed herein, the at least one SS18::SSX fusion junction epitope comprises the amino acid sequence of SEQ ID NO: 1 with 1, 2, 3, 4, 5, 6, 7, 8, 9, or 10 amino acid residues inserted at the N-terminus, the C-terminus, or both. In other embodiments of the vaccine disclosed herein, the at least one SS18::SSX fusion junction epitope comprises the amino acid sequence of SEQ ID NO: 1 with 1, 2, 3, 4, 5, 6, 7, or 8 amino acid residues deleted at the N-terminus, or the C-terminus. Additionally or alternatively, in some embodiments, the at least one SS18::SSX fusion junction epitope comprises an amino acid sequence that has about 90%, about 91%, about 92%, about 93%, about 94%, about 95%, about 96%, about 97%, about 98%, or about 99% identity to SEQ ID NO: 1.

Additionally or alternatively, in some embodiments, the nucleic acid further comprises a 5′ untranslated region and a 3′ untranslated region that has been optimized for enhanced translation efficiency. Additionally or alternatively, in some embodiments, the polypeptide or nucleic acid encoding the same further comprises a leader peptide at its N-terminus and an MHC class I trafficking signal (MITD) at its C terminus. In some embodiments, the MITD comprises the nucleic acid sequence 5′-gga tcc acc atg gag tcg cgc ggt cgc cgt tgt ccc gaa atg-3′ (SEQ ID NO: 12).

Additionally or alternatively, in some embodiments of the vaccine disclosed herein, the at least one CTA epitope is derived from one or more of NY-ESO-1, PRAME, or MAGEA4. In some embodiments, the at least one CTA epitope comprises a NY-ESO-1 epitope comprising one or more amino acid sequences selected from among SLLMWITQC (SEQ ID NO: 2), LLMWITQCF (SEQ ID NO: 3), APRGPHGGAASGL (SEQ ID NO: 4), LEFYLAMPF (SEQ ID NO: 5) and ARGPESRLL (SEQ ID NO: 6). In certain embodiments, the at least one CTA epitope comprises a MAGE-A4 epitope comprising one or more amino acid sequences selected from among EVDPASNTY (SEQ ID NO: 7), GVYDGREHTV (SEQ ID NO: 8), and NYKRCFPVI (SEQ ID NO: 9). Additionally or alternatively, in some embodiments, the at least one CTA epitope comprises a PRAME epitope comprising the amino acid sequence of ALYVDSLFFL (SEQ ID NO: 10).

Additionally or alternatively, in some embodiments, the oligomer of the at least one CTA epitope may include any one or more of the peptides comprising the amino acid sequence of any one or more of SEQ ID NOs: 2-10 with 1, 2, 3, 4, 5, 6, 7, 8, 9, or 10 amino acid residues inserted at the N-terminus, the C-terminus, or both. Additionally or alternatively, in certain embodiments, the oligomer of the at least one CTA epitope may include any one or more of the peptides comprising the amino acid sequence of any one or more of SEQ ID NOs: 2-10 with 1, 2, 3, 4, or 5 amino acid residues deleted at the N-terminus, or the C-terminus. In some embodiments, the oligomer of the at least one CTA epitope may include one or more peptides having about 90%, about 91%, about 92%, about 93%, about 94%, about 95%, about 96%, about 97%, about 98%, or about 99% identity to any one of the sequences of SEQ ID NOs: 2-10.

Additionally or alternatively, in certain embodiments of the vaccine disclosed herein, the oligomer is a dimer, a trimer, a tetramer, a pentamer, a hexamer, a heptamer, an octamer, a nonamer, a decamer, an 11-mer, a 12-mer, a 13-mer, a 14-mer, a 15-mer, a 16-mer, a 17-mer, an 18-mer, a 19-mer, a 20-mer, a 21-mer, a 22-mer, a 23-mer, a 24-mer, a 25-mer, a 26-mer, a 27-mer, a 28-mer, a 29-mer, or a 30-mer. In other embodiments, the oligomer of the at least one CTA epitope comprises 2, 3, 4, 5, 6, 7, 8, or 9 distinct CTA epitopes.

In any of the preceding embodiments of the vaccine disclosed herein, the oligomer of the at least one CTA epitope comprises tandem repeats of a single CTA epitope, optionally wherein the tandem repeats of the single CTA epitope are linked via a linker. In other embodiments, the oligomer of the at least one CTA epitope comprises at least 2 distinct CTA epitopes, optionally wherein the at least 2 distinct CTA epitopes are linked via a linker. The linker may be a cleavable peptide linker or a rigid peptide linker. Examples of linker include, but are not limited to, a GS4 linker (SEQ ID NO: 13), a GS3 linker (SEQ ID NO: 14), a P2A linker, a T2A linker, an E2A linker, a F2A linker, a BmCPV2A linker, an AAY linker, a GPGPG linker (SEQ ID NO: 15), an EAAAK linker (SEQ ID NO: 16), a HEYGAEALERAG linker (SEQ ID NO: 17), a KK linker, or an RVRR linker (SEQ ID NO: 18). Additionally or alternatively, in certain embodiments, the oligomer of the at least one CTA epitope further comprises one or more sequences of any one of SEQ ID NOs: 2-10.

In any and all embodiments of the vaccine disclosed herein, the SS18::SSX fusion junction epitope and/or the at least one CTA epitope are presented by one or more of HLA-A*02:01, HLA-A*03:01, HLA-A*11:01, HLA-B*07:02, HLA-C*04:01, HLA-C*12:03, HLA-A *01:01, HLA-A *24:02, HLA-A *24:10, HLA-A *01:02, HLA-A *68:01, HLA-A *26:01, HLA-B*57:02, HLA-B*14:02, HLA-B* 35:01, HLA-B*38:01, HLA-B*41:01, HLA-B*49:01, HLA-B*39:06, HLA-B* 18:02, HLA-B*40:02, HLA-B*35:02, HLA-B*51:01, HLA-B*57:01, HLA-B*18:01, HLA-B*44:05, HLA-C*17:01, HLA-C*15:04, HLA-C*06:02, HLA-C*07:01, HLA-C* 02:02, HLA-C*03:04, HLA-C*07:02, HLA-C*08:02, or HLA-C*07:02.

Caps. Traditionally, the basic components of an mRNA molecule include at least a coding region, a 5′ UTR, a 3′ UTR, a 5′ capping region and a poly-A tail. The 5′ capping region may include a naturally occurring cap, a synthetic cap or an optimized cap. Non-limiting examples of optimized caps include the caps taught by Rhoads in U.S. Pat. No. 7,074,596 and International Patent Publication No. WO2008157668, WO2009149253 and WO2013103659, the contents of each of which are herein incorporated by reference in its entirety.

The capping region may comprise a single cap or a series of nucleotides forming the cap. In this embodiment the capping region may be from 1 to 10, e.g. 2-9, 3-8, 4-7, 1-5, 5-10, or at least 2, or 10 or fewer nucleotides in length. In some embodiments, the cap is absent.

The 5′ cap structure of a natural mRNA is involved in nuclear export, increasing mRNA stability and binds the mRNA Cap Binding Protein (CBP), which is responsible for mRNA stability in the cell and translation competency through the association of CBP with poly(A) binding protein to form the mature cyclic mRNA species. The cap further assists the removal of 5′ proximal introns removal during mRNA splicing.

Endogenous mRNA molecules may be 5′-end capped generating a 5′-ppp-5′-triphosphate linkage between a terminal guanosine cap residue and the 5′-terminal transcribed sense nucleotide of the mRNA molecule. This 5′-guanylate cap may then be methylated to generate an N7-methyl-guanylate residue. The ribose sugars of the terminal and/or anteterminal transcribed nucleotides of the 5′ end of the mRNA may optionally also be 2′-O-methylated. 5′-decapping through hydrolysis and cleavage of the guanylate cap structure may target a nucleic acid molecule, such as an mRNA molecule, for degradation.

In some embodiments, the nucleic acids encoding one or more of the recombinant SS18::SSX fusion peptide epitopes and at least one cancer-testis antigen (CTA) epitope of the present disclosure may be designed to incorporate a cap moiety. Modifications to the nucleic acids of the present disclosure may generate a non-hydrolyzable cap structure preventing decapping and thus increasing mRNA half-life. Because cap structure hydrolysis requires cleavage of 5′-ppp-5′ phosphorodiester linkages, modified nucleotides may be used during the capping reaction. For example, a Vaccinia Capping Enzyme from New England Biolabs (Ipswich, MA) may be used with a-thio-guanosine nucleotides according to the manufacturer's instructions to create a phosphorothioate linkage in the 5′-ppp-5′ cap. Additional modified guanosine nucleotides may be used such as a-methyl-phosphonate and seleno-phosphate nucleotides.

Additional modifications include, but are not limited to, 2′-O-methylation of the ribose sugars of 5′-terminal and/or 5′-anteterminal nucleotides of the polynucleotide on the 2′-hydroxyl group of the sugar ring. Multiple distinct 5′-cap structures can be used to generate the 5′-cap of a nucleic acid molecule, such as a polynucleotide which functions as an mRNA molecule.

Cap analogs, which herein are also referred to as synthetic cap analogs, chemical caps, chemical cap analogs, or structural or functional cap analogs, differ from natural (i.e. endogenous, wild-type or physiological) 5′-caps in their chemical structure, while retaining cap function. Cap analogs may be chemically (i.e. non-enzymatically) or enzymatically synthesized and/or linked to the nucleic acids disclosed herein.

For example, the Anti-Reverse Cap Analog (ARCA) cap contains two guanines linked by a 5′-5′-triphosphate group, wherein one guanine contains an N7 methyl group as well as a 3′-O-methyl group (i.e., N7,3′-O-dimethyl-guanosine-5′-triphosphate-5′-guanosine (m G-3′mppp-G; which may equivalently be designated 3′ O-Me-m7G(5′)ppp(5′)G). The 3′-0 atom of the other, unmodified, guanine becomes linked to the 5′-terminal nucleotide of the capped polynucleotide. The N7- and 3′-O-methylated guanine provides the terminal moiety of the capped polynucleotide.

Another exemplary cap is mCAP, which is similar to ARCA but has a 2′-O-methyl group on guanosine (i.e., N7,2′-O-dimethyl-guanosine-5′-triphosphate-5′-guanosine, m Gm-ppp-G).

In one embodiment, the cap is a dinucleotide cap analog. As a non-limiting example, the dinucleotide cap analog may be modified at different phosphate positions with a boranophosphate group or a phosphoroselenoate group such as the dinucleotide cap analogs described in U.S. Pat. No. 8,519,110, the contents of which are herein incorporated by reference in its entirety.

In another embodiment, the cap is a cap analog is a N7-(4-chlorophenoxyethyl) substituted dinucleotide form of a cap analog known in the art and/or described herein. Non-limiting examples of a N7-(4-chlorophenoxyethyl) substituted dinucleotide form of a cap analog include a N7-(4-chlorophenoxyethyl)-G(5′)ppp(5′)G and a N7-(4-chlorophenoxyethyl)-m^{3″}°G(5′)ppp(5′)G cap analog (See e.g., the various cap analogs and the methods of synthesizing cap analogs described in Kore et al. Bioorganic & Medicinal Chemistry 2013 21:4570-4574; the contents of which are herein incorporated by reference in its entirety). In another embodiment, a cap analog of the present disclosure is a 4-chloro/bromophenoxyethyl analog.

While cap analogs allow for the concomitant capping of a polynucleotide or a region thereof, in an in vitro transcription reaction, up to 20% of transcripts can remain uncapped. This, as well as the structural differences of a cap analog from an endogenous 5′-cap structures of nucleic acids produced by the endogenous, cellular transcription machinery, may lead to reduced translational competency and reduced cellular stability. Nucleic acids encoding one or more of the recombinant SS18::SSX fusion peptide epitopes and at least one cancer-testis antigen (CTA) epitope of the present disclosure may also be capped post-manufacture (whether IVT or chemical synthesis), using enzymes, in order to generate more authentic 5′-cap structures. As used herein, the phrase “more authentic” refers to a feature that closely mirrors or mimics, either structurally or functionally, an endogenous or wild type feature. That is, a “more authentic” feature is better representative of an endogenous, wild-type, natural or physiological cellular function and/or structure as compared to synthetic features or analogs, etc., of the prior art, or which outperforms the corresponding endogenous, wild-type, natural or physiological feature in one or more respects. Non-limiting examples of more authentic 5′cap structures of the present disclosure are those which, among other things, have enhanced binding of cap binding proteins, increased half life, reduced susceptibility to 5′ endonucleases and/or reduced 5′decapping, as compared to synthetic 5′cap structures known in the art (or to a wild-type, natural or physiological 5′cap structure). For example, recombinant Vaccinia Virus Capping Enzyme and recombinant 2′-O-methyltransferase enzyme can create a canonical 5′-5′-triphosphate linkage between the 5′-terminal nucleotide of a polynucleotide and a guanine cap nucleotide wherein the cap guanine contains an N7 methylation and the 5′-terminal nucleotide of the mRNA contains a 2′-O-methyl. Such a structure is termed the Capl structure. This cap results in a higher translational-competency and cellular stability and a reduced activation of cellular pro-inflammatory cytokines, as compared, e.g., to other 5′cap analog structures known in the art. Cap structures include, but are not limited to, 7mG(5′)ppp(5′)N,pN2p (cap 0), 7mG(5′)ppp(5′)NlmpNp (cap 1), and 7mG(5′)-ppp(5′)NlmpN2mp (cap 2).

As a non-limiting example, capping chimeric polynucleotides post-manufacture may be more efficient as nearly 100% of the chimeric polynucleotides may be capped. This is in contrast to −80% when a cap analog is linked to a chimeric polynucleotide in the course of an in vitro transcription reaction.

According to the present disclosure, 5′ terminal caps may include endogenous caps or cap analogs. According to the present disclosure, a 5′ terminal cap may comprise a guanine analog. Useful guanine analogs include, but are not limited to, inosine, Nl-methyl-guanosine, 2′fluoro-guanosine, 7-deaza-guanosine, 8-oxo-guanosine, 2-amino-guanosine, LNA-guanosine, and 2-azido-guanosine.

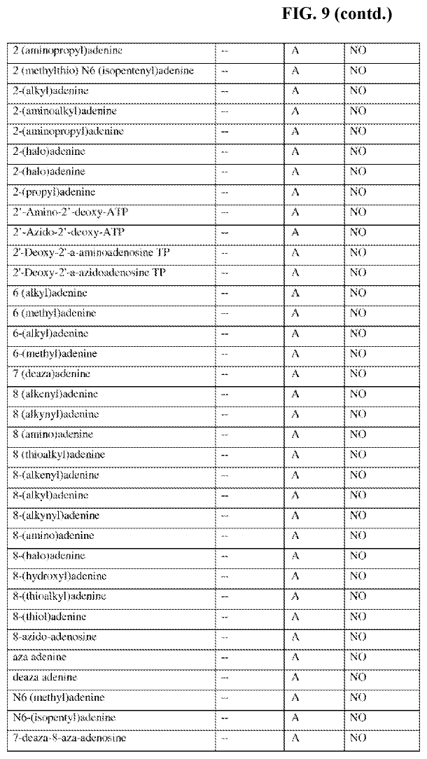

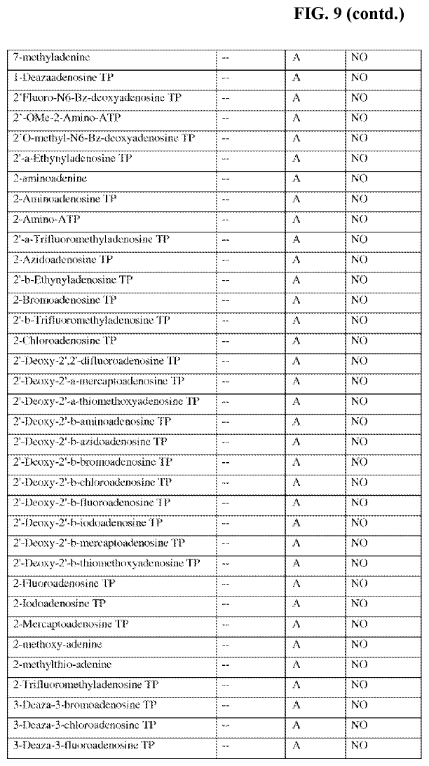

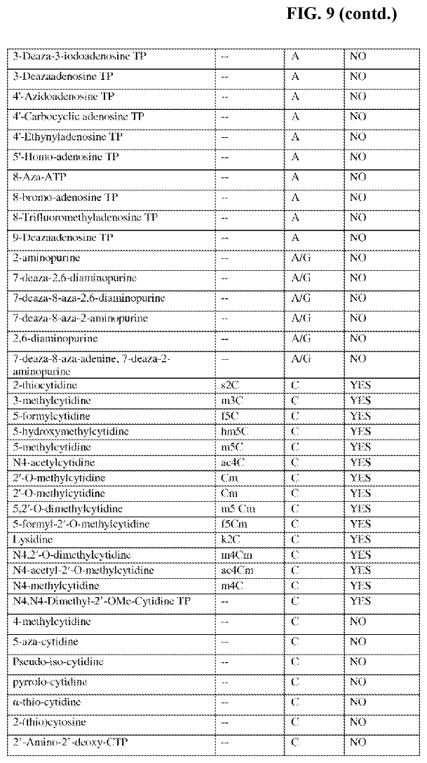

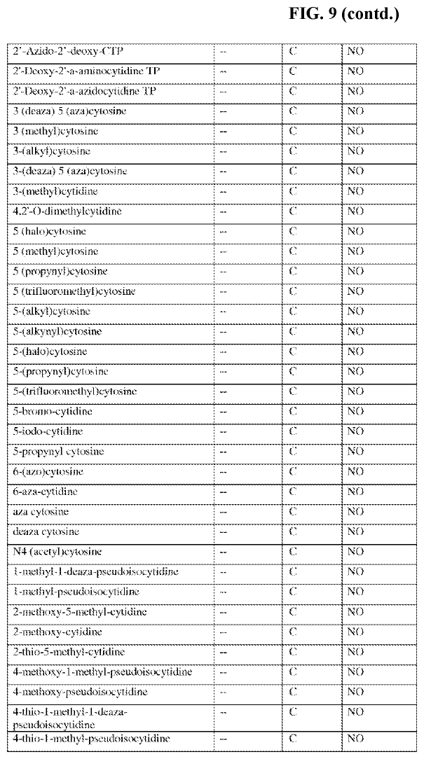

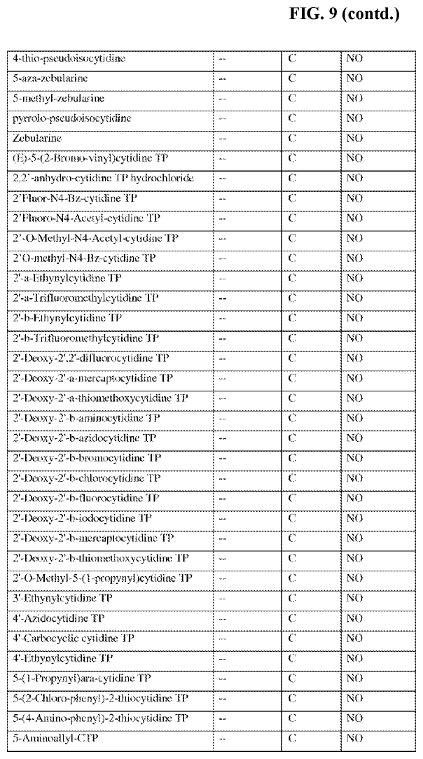

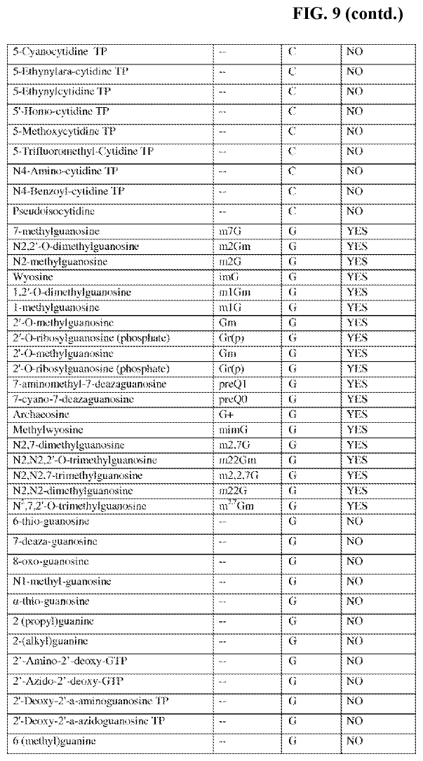

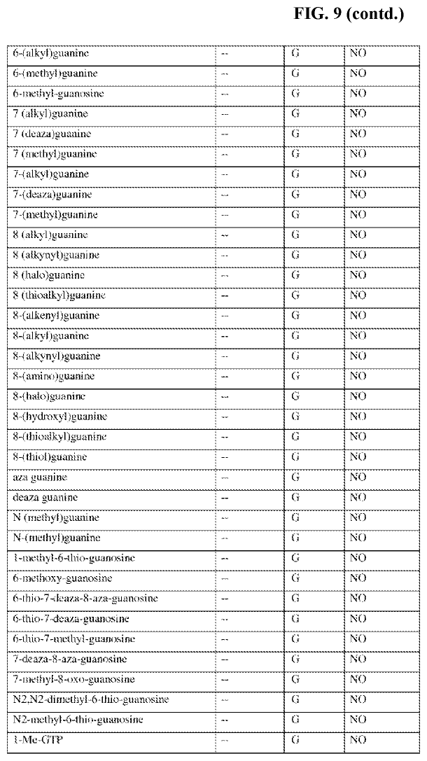

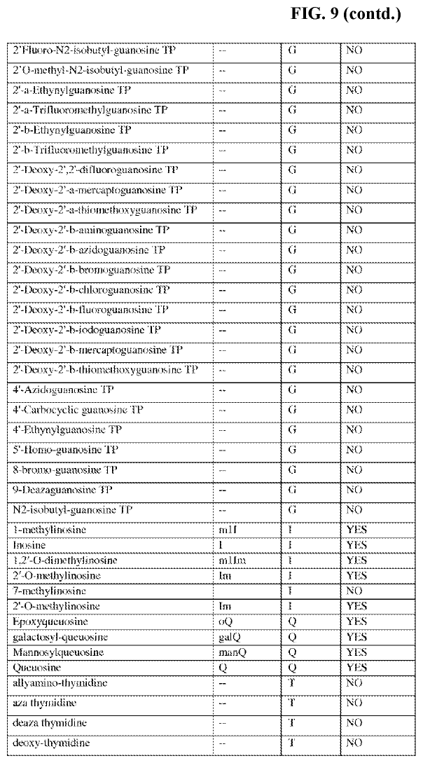

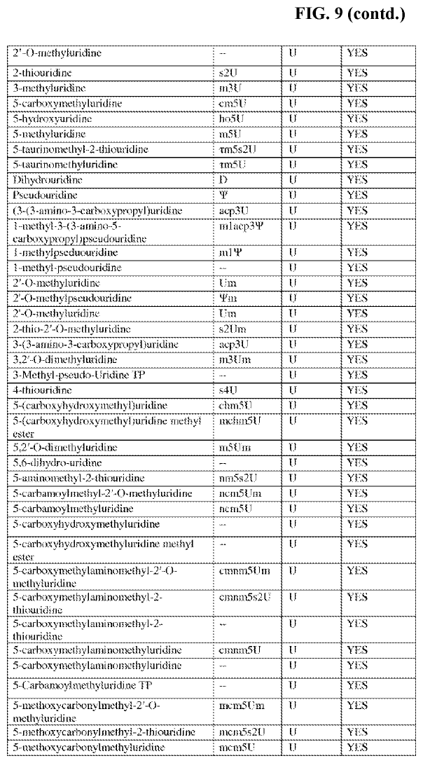

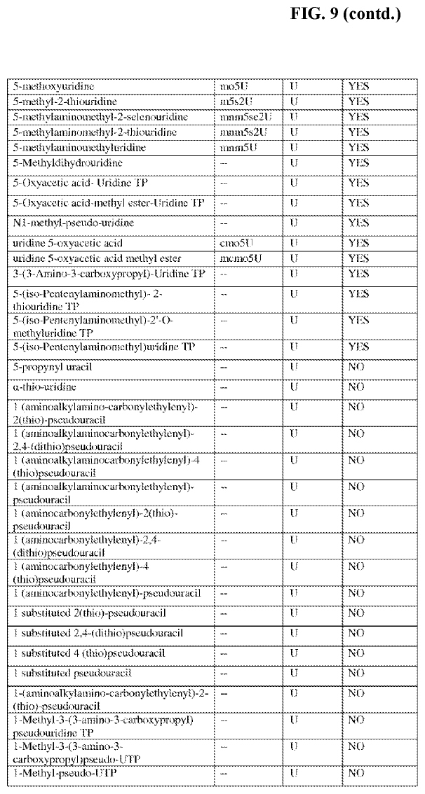

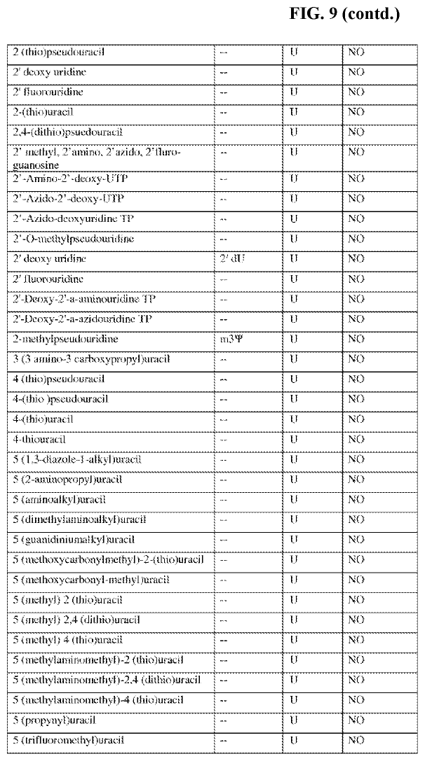

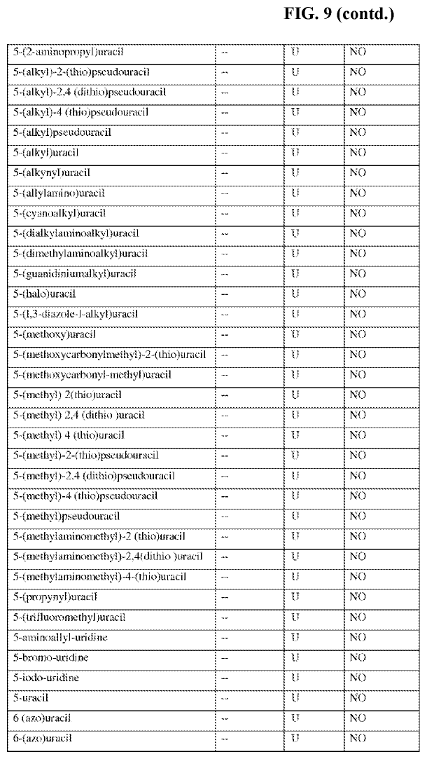

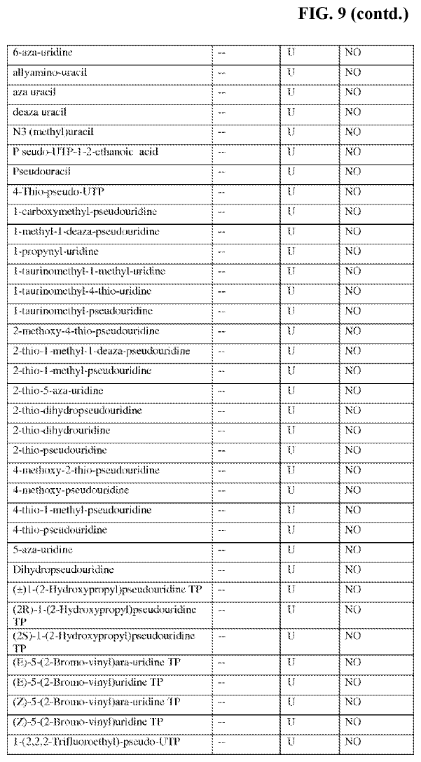

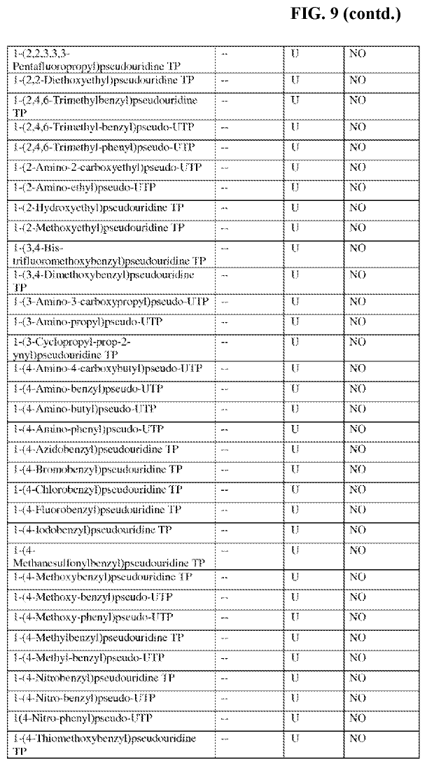

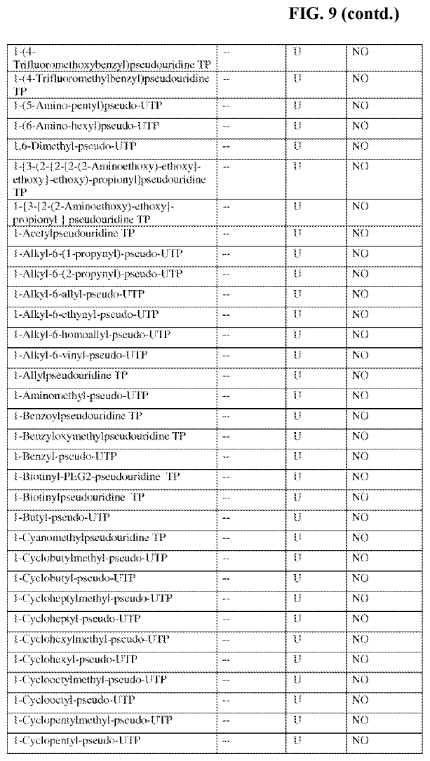

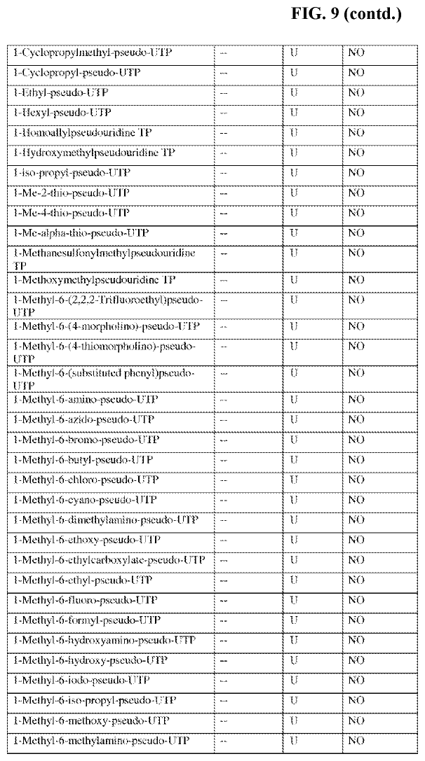

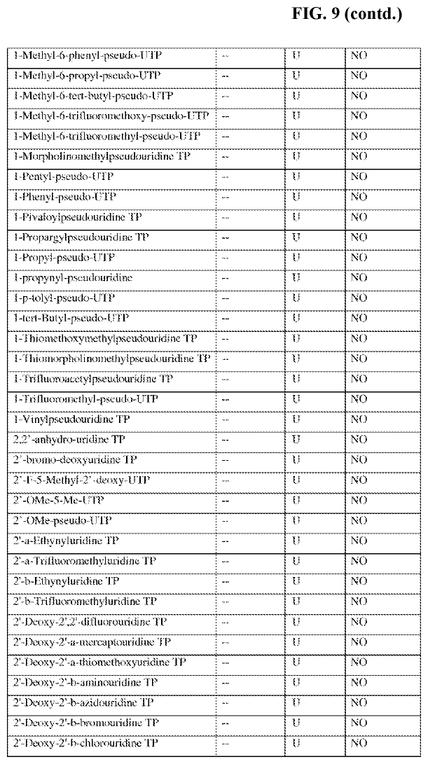

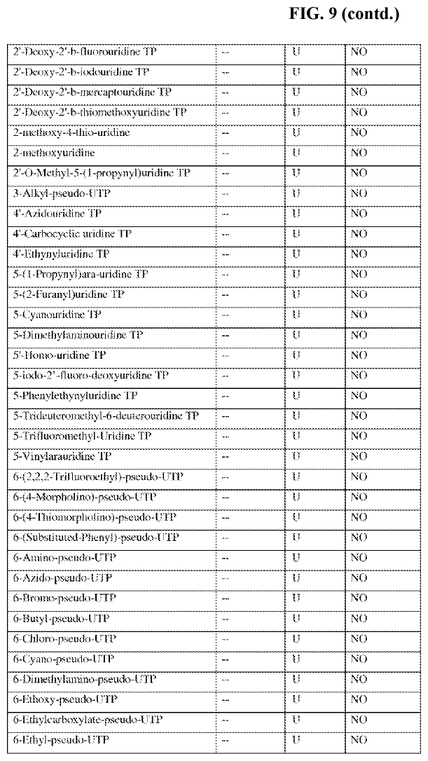

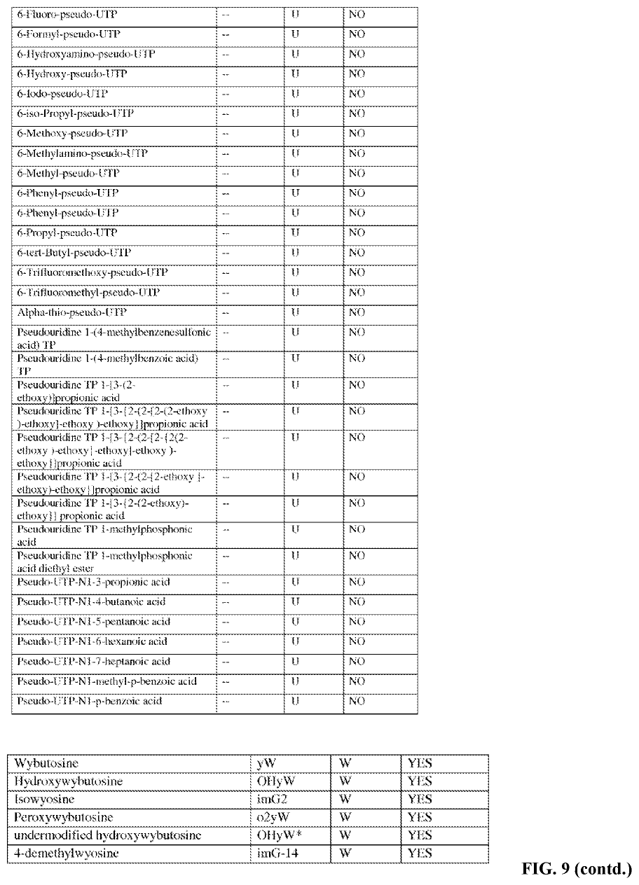

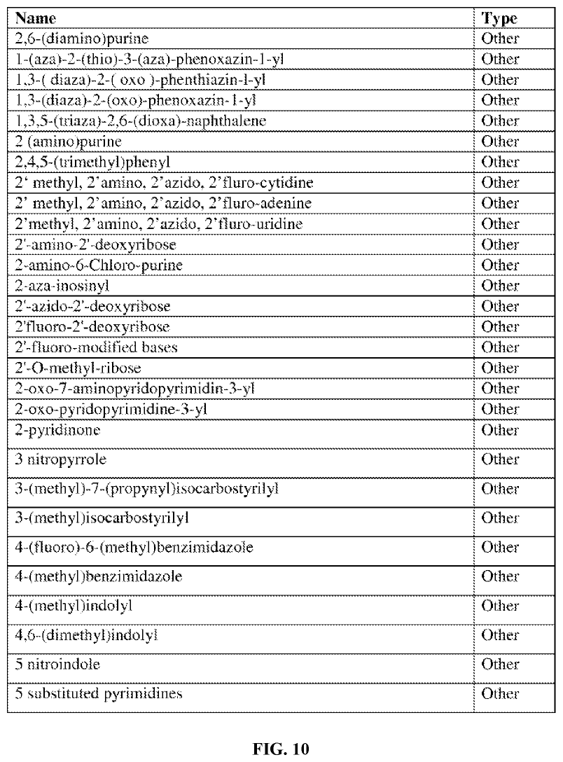

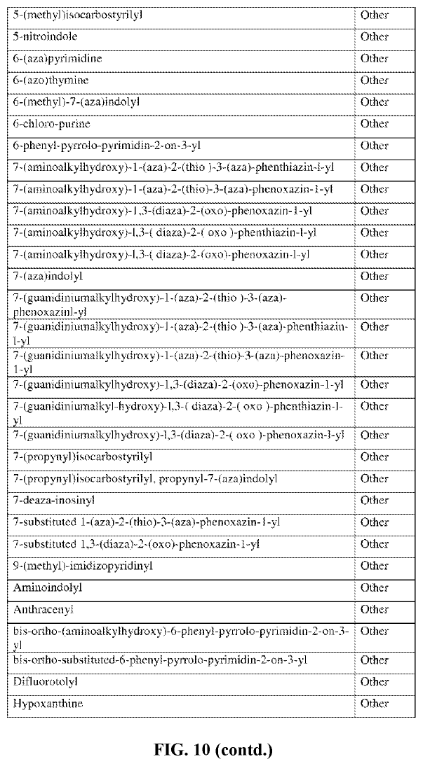

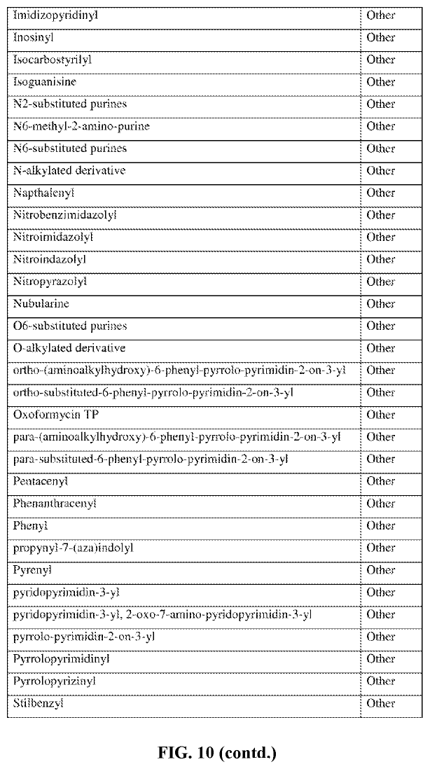

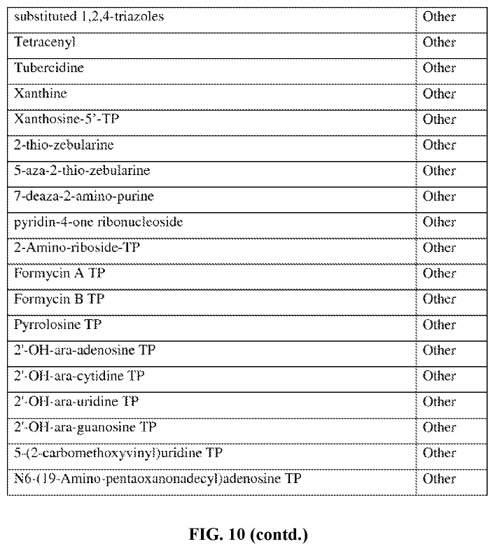

Chemical Modifications. In some embodiments, the nucleic acids (e.g. mRNA, cDNA) encoding one or more of the recombinant SS18::SSX fusion peptide epitopes and at least one cancer-testis antigen (CTA) epitope of the present disclosure include a chemical modification and the chemical modification is selected from any of those listed in FIGS. 9-10. In some embodiments, the chemical modification is selected from the group consisting of pseudouridine, N1-methylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, Dihydropseudouridine, 5-methoxyuridine and 2′-O-methyl uridine. In some embodiments, the RNA polynucleotide further comprises a second chemical modification wherein said second chemical modification is selected from any of those listed in FIGS. 9-10. In some embodiments, the combination of first and second chemical modification is selected from those listed in Table A.

TABLE A

| Modified | |
|---|---|
| Nucleotide | Modified Nucleotide Combination |
| α-thio-cytidine | α-thio-cytidine/5-iodo-uridine |
| | α-thio-cytidine/N1-methyl-pseudouridine |
| | α-thio-cytidine/α-thio-uridine |
| | α-thio-cytidine/5-methyl-uridine |
| | α-thio-cytidine/pseudo-uridine |
| | about 50% of the cytosines are α-thio-cytidine |
| Pseudoisocytidine | pseudoisocytidine/5-iodo-uridine |
| | pseudoisocytidine/N1-methyl-pseudouridine |
| | pseudoisocytidine/α-thio-uridine |
| | pseudoisocytidine/5-methyl-uridine |
| | pseudoisocytidine/pseudouridine |
| | about 25% of cytosines are pseudoisocytidine |
| | pseudoisocytidine/about 50% of uridines are N1- |
| | methyl-pseudouridine and about 50% of uridines are |
| | pseudouridine |
| | pseudoisocytidine/about 25% of uridines are N1- |
| | methyl-pseudouridine and about 25% of uridines are |
| | pseudouridine |
| pyrrolo-cytidine | pyrrolo-cytidine/5-iodo-uridine |
| | pyrrolo-cytidine/N1-methyl-pseudouridine |
| | pyrrolo-cytidine/α-thio-uridine |
| | pyrrolo-cytidine/5-methyl-uridine |
| | pyrrolo-cytidine/pseudouridine |
| | about 50% of the cytosines are pyrrolo-cytidine |
| 5-methyl-cytidine | 5-methyl-cytidine/5-iodo-uridine |
| | 5-methyl-cytidine/N1-methyl-pseudouridine |
| | 5-methyl-cytidine/α-thio-uridine |
| | 5-methyl-cytidine/5-methyl-uridine |
| | 5-methyl-cytidine/pseudouridine |
| | about 25% of cytosines are 5-methyl-cytidine |
| | about 50% of cytosines are 5-methyl-cytidine |
| | 5-methyl-cytidine/5-methoxy-uridine |
| | 5-methyl-cytidine/5-bromo-uridine |
| | 5-methyl-cytidine/2-thio-uridine |
| | 5-methyl-cytidine/about 50% of uridines are 2-thio- |
| | uridine |
| | about 50% of uridines are 5-methyl-cytidine/about |
| | 50% of uridines are 2-thio-uridine |
| N4-acetyl-cytidine | N4-acetyl-cytidine/5-iodo-uridine |
| | N4-acetyl-cytidine/N1-methyl-pseudouridine |
| | N4-acetyl-cytidine/α-thio-uridine |
| | N4-acetyl-cytidine/5-methyl-uridine |
| | N4-acetyl-cytidine/pseudouridine |
| | about 50% of cytosines are N4-acetyl-cytidine |
| | about 25% of cytosines are N4-acetyl-cytidine |
| | N4-acetyl-cytidine/5-methoxy-uridine |
| | N4-acetyl-cytidine/5-bromo-uridine |
| | N4-acetyl-cytidine/2-thio-uridine |
| | about 50% of cytosines are N4-acetyl-cytidine/about |
| | 50% of uridines are 2-thio-uridine |

The nucleic acids (e.g. mRNA, cDNA) encoding one or more of the recombinant SS 18:: SSX fusion peptide epitopes and at least one cancer-testis antigen (CTA) epitope of the present disclosure can include any useful linker between the nucleosides. Such linkers, including backbone modifications are given in Table B:

TABLE B

| Name | TYPE |
|---|---|
| 3′-alkylene phosphonates | Linker |
| 3′-amino phosphoramidate | Linker |
| alkene containing backbones | Linker |
| Aminoalkylphosphoramidates | Linker |
| Aminoalkylphosphotriesters | Linker |
| Boranophosphates | Linker |
| —CH2-0-N(CH3)—CH2— | Linker |
| —CH2—N(CH3)—N(CH3)—CH2— | Linker |
| —CH2—NH—CH2— | Linker |
| chiral phosphonates | Linker |
| chiral phosphorothioates | Linker |
| formacetyl and thioformacetyl backbones | Linker |
| methylene (methylimino) | Linker |
| methylene formacetyl and thioformacetyl backbones | Linker |
| methyleneimino and methylenehydrazino backbones | Linker |
| morpholino linkages | Linker |
| —N(CH3)—CH2—CH2— | Linker |
| oligonucleosides with heteroatom internucleoside linkage | Linker |
| Phosphinates | Linker |
| phosphoramidates | Linker |
| Phosphorodithioates | Linker |
| phosphorothioate internucleoside linkages | Linker |
| Phosphorothioates | Linker |
| Phosphotriesters | Linker |
| PNA | Linker |
| siloxane backbones | Linker |
| sulfamate backbones | Linker |
| sulfide sulfoxide and sulfone backbones | Linker |
| sulfonate and sulfonamide backbones | Linker |
| Thionoalkylphosphonates | Linker |
| Thionoalkylphosphotriesters | Linker |
| Thionophosphoramidates | Linker |

The nucleic acids (e.g. mRNA, cDNA) encoding one or more of the recombinant SS18::SSX fusion peptide epitopes and at least one cancer-testis antigen (CTA) epitope of the present disclosure can include any useful modification, such as to the sugar, the nucleobase, or the internucleoside linkage (e.g. to a linking phosphate/to a phosphodiester linkage/to the phosphodiester backbone). One or more atoms of a pyrimidine nucleobase may be replaced or substituted with optionally substituted amino, optionally substituted thiol, optionally substituted alkyl (e.g., methyl or ethyl), or halo (e.g., chloro or fluoro). In certain embodiments, modifications (e.g., one or more modifications) are present in each of the sugar and the internucleoside linkage. Modifications according to the present disclosure may be modifications of ribonucleic acids (RNAs) to deoxyribonucleic acids (DNAs), threose nucleic acids (TNAs), glycol nucleic acids (GNAs), peptide nucleic acids (PNAs), locked nucleic acids (LNAs) or hybrids thereof). Additional modifications are described herein.

Non-natural modified nucleotides may be introduced to polynucleotides, e.g., of the nucleic acids (e.g. mRNA, cDNA) encoding one or more of the recombinant SS18::SSX fusion peptide epitopes and at least one cancer-testis antigen (CTA) epitope of the present disclosure, or nucleic acids during synthesis or post-synthesis of the chains to achieve desired functions or properties. The modifications may be on internucleotide lineage, the purine or pyrimidine bases, or sugar. The modification may be introduced at the terminal of a chain or anywhere else in the chain; with chemical synthesis or with a polymerase enzyme. For example, hexitol nucleic acids (HNAs) are nuclease resistant and provide strong hybridization to RNA. Short messenger RNAs (mRNAs) with hexitol residues in two codons have been constructed (Lavrik et al., Biochemistry, 40, 11777-11784 (2001), the contents of which are incorporated herein by reference in their entirety). The antisense effects of a chimeric HNA gapmer oligonucleotide comprising a phosphorothioate central sequence flanked by 5′ and 3′ HNA sequences have also been studied (See e.g., Kang et al., Nucleic Acids Research, vol. 32(4), 4411-4419 (2004), the contents of which are incorporated herein by reference in their entirety). The preparation and uses of modified nucleotides comprising 6-member rings in RNA interference, antisense therapy or other applications are disclosed in US Pat. Application No. 2008/0261905, US Pat. Application No. 2010/0009865, and PCT Application No. WO97/30064 to Herdewijn et al.; the contents of each of which are herein incorporated by reference in their entireties). Modified nucleic acids and their synthesis are disclosed in copending PCT applications No. PCT/US2012/058519 (Attorney Docket Number M09), the contents of which are incorporated herein by reference for their entirety. The synthesis and strategy of modified polynucleotides is reviewed by Verma and Eckstein in Annual Review of Biochemistry, vol. 76, 99-134 (1998), the contents of which are incorporated herein by reference in their entirety.

Either enzymatic or chemical ligation methods can be used to conjugate polynucleotides or their regions with different functional blocks, such as fluorescent labels, liquids, nanoparticles, delivery agents, etc. The conjugates of polynucleotides and modified polynucleotides are reviewed by Goodchild in Bioconjugate Chemistry, vol. 1(3), 165-187 (1990), the contents of which are incorporated herein by reference in their entirety. U.S. Pat. Nos. 6,835,827 and 6,525,183 to Vinayak et al. (the contents of each of which are herein incorporated by reference in their entireties) teach synthesis of labeled oligonucleotides using a labeled solid support.

In certain embodiments, it may desirable to intracellularly degrade a polynucleotide introduced into the cell. For example, degradation of a polynucleotide may be preferable if precise timing of protein production is desired. Thus, in some embodiments, the present disclosure provides a polynucleotide containing a degradation domain, which is capable of being acted on in a directed manner within a cell. Any of the regions of the polynucleotides may be chemically modified as taught herein or as taught in International Application Number PCT/2012/058519 filed Oct. 3, 2012 and U.S. Provisional Application No. 61/837,297 filed Jun. 20, 2013 the contents of each of which are incorporated herein by reference in its entirety.

Modified Polynucleotide Molecules. The present disclosure also includes building blocks, e.g., modified ribonucleosides, and modified ribonucleotides, of nucleic acids (e.g. mRNA, cDNA) encoding the one or more of the recombinant SS18::SSX fusion peptide epitopes and at least one cancer-testis antigen (CTA) epitope of the present disclosure. For example, these building blocks can be useful for preparing the polynucleotides of the present disclosure. Such building blocks are taught in International Application Number PCT/2012/058519 filed Oct. 3, 2012 and U.S. Provisional Application No. 61/837,297 filed Jun. 20, 2013 the contents of each of which are incorporated herein by reference in its entirety.

Modifications on the Sugar. The modified nucleosides and nucleotides (e.g., building block molecules), which may be incorporated into a polynucleotide (e.g., RNA or mRNA, as described herein), can be modified on the sugar of the ribonucleic acid. For example, the 2′ hydroxyl group (OH) can be modified or replaced with a number of different substituents. Exemplary substitutions at the 2′-position include, but are not limited to, H, halo, optionally substituted C₁₋₆ alkyl; optionally substituted C₁₋₆ alkoxy; optionally substituted C₆₋₁₀ aryloxy; optionally substituted C₃₋₈ cycloalkyl; optionally substituted C₃₋₈ cycloalkoxy; optionally substituted C₆₋₁₀ aryloxy; optionally substituted C₆₋₁₀ aryl-C₁₋₆ alkoxy, optionally substituted C₁₋₁₂ (heterocyclyl)oxy; a sugar (e.g., ribose, pentose, or any described herein); a polyethyleneglycol (PEG), —O(CH₂CH₂O)ₙCH₂CH₂OR, where R is H or optionally substituted alkyl, and n is an integer from 0 to 20 (e.g., from 0 to 4, from 0 to 8, from 0 to 10, from 0 to 16, from 1 to 4, from 1 to 8, from 1 to 10, from 1 to 16, from 1 to 20, from 2 to 4, from 2 to 8, from 2 to 10, from 2 to 16, from 2 to 20, from 4 to 8, from 4 to 10, from 4 to 16, and from 4 to 20); “locked” nucleic acids (LNA) in which the 2′-hydroxyl is connected by a C₁₋₆ alkylene or C₁₋₆ heteroalkylene bridge to the 4′-carbon of the same ribose sugar, where exemplary bridges included methylene, propylene, ether, or amino bridges; aminoalkyl, as defined herein; aminoalkoxy, as defined herein; amino as defined herein; and amino acid, as defined herein Generally, RNA includes the sugar group ribose, which is a 5-membered ring having an oxygen. Exemplary, non-limiting modified nucleotides include replacement of the oxygen in ribose (e.g., with S, Se, or alkylene, such as methylene or ethylene); addition of a double bond (e.g., to replace ribose with cyclopentenyl or cyclohexenyl); ring contraction of ribose (e.g., to form a 4-membered ring of cyclobutane or oxetane); ring expansion of ribose (e.g., to form a 6- or 7-membered ring having an additional carbon or heteroatom, such as for anhydrohexitol, altritol, mannitol, cyclohexanyl, cyclohexenyl, and morpholino that also has a phosphoramidate backbone); multicyclic forms (e.g., tricyclo; and “unlocked” forms, such as glycol nucleic acid (GNA) (e.g., R-GNA or S-GNA, where ribose is replaced by glycol units attached to phosphodiester bonds), threose nucleic acid (TNA, where ribose is replace with a-L-threofuranosyl-(3′→2′)), and peptide nucleic acid (PNA, where 2-amino-ethyl-glycine linkages replace the ribose and phosphodiester backbone). The sugar group can also contain one or more carbons that possess the opposite stereochemical configuration than that of the corresponding carbon in ribose. Thus, a polynucleotide molecule can include nucleotides containing, e.g., arabinose, as the sugar. Such sugar modifications are taught International Application Number PCT/2012/058519 filed Oct. 3, 2012 and U.S. Provisional Application No. 61/837,297 filed Jun. 20, 2013, the contents of each of which are incoroporated herein by reference in its entirety.

Modifications on the Nucleobase. The present disclosure provides for modified nucleosides and nucleotides. As described herein “nucleoside” is defined as a compound containing a sugar molecule (e.g., a pentose or ribose) or a derivative thereof in combination with an organic base (e.g., a purine or pyrimidine) or a derivative thereof (also referred to herein as “nucleobase”). As described herein, “nucleotide” is defined as a nucleoside including a phosphate group. The modified nucleotides may by synthesized by any useful method, as described herein (e.g., chemically, enzymatically, or recombinantly to include one or more modified or non-natural nucleosides). The polynucleotides may comprise a region or regions of linked nucleosides. Such regions may have variable backbone linkages. The linkages may be standard phosphoester linkages, in which case the polynucleotides would comprise regions of nucleotides. The modified nucleotide base pairing encompasses not only the standard adenosine-thymine, adenosine-uracil, or guanosine-cytosine base pairs, but also base pairs formed between nucleotides and/or modified nucleotides comprising nonstandard or modified bases, wherein the arrangement of hydrogen bond donors and hydrogen bond acceptors permits hydrogen bonding between a non-standard base and a standard base or between two complementary non-standard base structures. One example of such non-standard base pairing is the base pairing between the modified nucleotide inosine and adenine, cytosine or uracil.

The modified nucleosides and nucleotides can include a modified nucleobase. Examples of nucleobases found in RNA include, but are not limited to, adenine, guanine, cytosine, and uracil. Examples of nucleobase found in DNA include, but are not limited to, adenine, guanine, cytosine, and thymine. Such modified nucleobases (including the distinctions between naturally occurring and non-naturally occurring) are taught in International Application Number PCT/2012/058519 filed Oct. 3, 2012 and U.S. Provisional Application No. 61/837,297 filed Jun. 20, 2013, the contents of each of which are incoroporated herein by reference in its entirety.

### Combinations of Modified Sugars, Nucleobases, and Internucleoside Linkages

The nucleic acids (e.g. mRNA, cDNA) encoding one or more of the recombinant SS18::SSX fusion peptide epitopes and at least one cancer-testis antigen (CTA) epitope of the present disclosure, can include a combination of modifications to the sugar, the nucleobase, and/or the internucleoside linkage. These combinations can include any one or more modifications described herein. Examples of modified nucleotides and modified nucleotide combinations are provided below in Table A. These combinations of modified nucleotides can be used to form the polynucleotides of the present disclosure. Unless otherwise noted, the modified nucleotides may be completely substituted for the natural nucleotides of the polynucleotides of the present disclosure. As a non-limiting example, the natural nucleotide uridine may be substituted with a modified nucleoside described herein. In another non-limiting example, the natural nucleotide uridine may be partially substituted (e.g., about 0.1%, 1%, 5%, 1, 1%, 20%, 25%, 30%, 35%, 40%, 45%, 50%, 55%, 60%, 65%, 70%, 75%, 80%, 85%, 90%, 95% or 99.9%) with at least one of the modified nucleoside disclosed herein. Any combination of base/sugar or linker may be incorporated into the polynucleotides of the present disclosure and such modifications are taught in International Application Number PCT/2012/058519 filed Oct. 3, 2012 and U.S. Provisional Application No. 61/837,297 filed Jun. 20, 2013 the contents of each of which are incorporated herein by reference in its entirety.

### Pharmaceutical Compositions and Preparations of the Present Technology

In any and all embodiments of the vaccine disclosed herein, the vaccine further comprises a pharmaceutically acceptable carrier selected from the group consisting of a cream, emulsion, gel, liposome, nanoparticle, or ointment. Disclosed herein are pharmaceutical compositions comprising recombinant SS18::SSX fusion peptide epitopes and at least one cancer-testis antigen (CTA) epitope, or nucleic acids (e.g. mRNA, cDNA) encoding the same, of the present disclosure that may contain a carrier or diluent, which can be a solvent or dispersion medium containing, for example, water, saline, Tris buffer, polyol (for example, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be effected by various antibacterial and antifungal agents and preservatives, for example, parabens, chlorobutanol, phenol, sorbic acid, thimerosal, and the like. In some embodiments, isotonic agents, for example, sugars or sodium chloride, and buffering agents are included. Prolonged absorption of the injectable compositions can be brought about by the use in the compositions of agents delaying absorption, for example, aluminum monostearate and gelatin or carrier molecules. Other excipients may include wetting or emulsifying agents. In general, excipients suitable for injectable preparations can be included as apparent to those skilled in the art.

Pharmaceutical compositions and preparations comprising recombinant SS18::SSX fusion peptide epitopes and at least one cancer-testis antigen (CTA) epitope, or nucleic acids (e.g., mRNA, cDNA) encoding the same, may be manufactured by means of conventional mixing, dissolving, granulating, emulsifying, encapsulating, entrapping or lyophilizing processes. Pharmaceutical compositions may be formulated in conventional manner using one or more physiologically acceptable carriers, diluents, excipients or auxiliaries that facilitate formulating preparations suitable for in vitro, in vivo, or ex vivo use. The compositions can be combined with one or more additional biologically active agents and may be formulated with a pharmaceutically acceptable carrier, diluent or excipient to generate pharmaceutical (including biologic) or veterinary compositions of the instant disclosure suitable for parenteral or intratumoral administration.

Many types of formulation are possible as is appreciated by those skilled in the art. The particular type chosen is dependent upon the route of administration chosen, as is well-recognized in the art. For example, systemic formulations will generally be designed for administration by injection, e.g., intravenous, as well as those designed for intratumoral delivery. In some embodiments, the systemic or intratumoral formulation is sterile.

Sterile injectable solutions are prepared by incorporating recombinant SS18::SSX fusion peptide epitopes and at least one cancer-testis antigen (CTA) epitope, or nucleic acids (e.g., mRNA, cDNA) encoding the same, in the required amount of the appropriate solvent with various other ingredients enumerated herein, as required, followed by suitable sterilization means. Generally, dispersions are prepared by incorporating the various sterilized active ingredients into a sterile vehicle that contains the basic dispersion medium and the required other ingredients from those enumerated above. In the case of sterile powders for the preparation of sterile injectable solutions, the preferred methods of preparation are vacuum drying and freeze-drying techniques, which yield a powder of plus any additional desired ingredient from a previously sterile-filtered solution thereof.

In some embodiments, the recombinant SS18::SSX fusion peptide epitopes and at least one cancer-testis antigen (CTA) epitope, or nucleic acids (e.g., mRNA, cDNA) encoding the same, compositions of the present disclosure may be formulated in aqueous solutions, or in physiologically compatible solutions or buffers such as Hanks's solution, Ringer's solution, mannitol solutions or physiological saline buffer. In certain embodiments, any of the recombinant SS18::SSX fusion peptide epitopes and at least one cancer-testis antigen (CTA) epitope, or nucleic acids (e.g., mRNA, cDNA) encoding the same, compositions may contain formulator agents, such as suspending, stabilizing, penetrating or dispersing agents, buffers, lyoprotectants or preservatives such as polyethylene glycol, polysorbate 80, 1-dodecylhexahydro-2H-azepin-2-one (laurocapran), oleic acid, sodium citrate, Tris HCl, dextrose, propylene glycol, mannitol, polysorbate poly-ethylenesorbitan monolaurate (Tween®-20), isopropyl myristate, benzyl alcohol, isopropyl alcohol, ethanol sucrose, trehalose and other such generally known in the art may be used in any of the compositions of the instant disclosure. (Pramanick et al., Pharma Times 45(3):65-76 (2013)).

The pharmaceutical composition according to the present disclosure may comprise an additional adjuvant. As used herein, an “adjuvant” refers to a substance that enhances, augments, or potentiates the host's immune response to tumor antigens. A typical adjuvant may be aluminum salts, such as aluminum hydroxide or aluminum phosphate, Quil A, bacterial cell wall peptidoglycans, virus-like particles, polysaccharides, toll-like receptors, nano-beads, etc. (Aguilar et al., Vaccine 25:3752-3762 (2007)).

### Liposomes, Lipoplexes, and Lipid Nanoparticle Compositions and Methods of

Preparation thereof

### Lipidoids

The synthesis of lipidoids has been extensively described and formulations containing these compounds are particularly suited for delivery of polynucleotides (see Mahon et al., Bioconjug Chem. 2010 21:1448-1454; Schroeder et al., J Intern Med. 2010 267:9-21; Akinc et al., Nat Biotechnol. 2008 26:561-569; Love et al., Proc Natl Acad Sci USA. 2010 107:1864-1869; Siegwart et al., Proc Natl Acad Sci USA. 2011 108:12996-3001; all of which are incorporated herein in their entireties).

While these lipidoids have been used to effectively deliver double stranded small interfering RNA molecules in rodents and non-human primates (see Akinc et al., Nat Biotechnol. 2008 26:561-569; Frank-Kamenetsky et al., Proc Natl Acad Sci USA. 2008 105:11915-11920; Akinc et al., Mol Ther. 2009 17:872-879; Love et al., Proc Natl Acad Sci USA. 2010 107:1864-1869; Leuschner et al., Nat Biotechnol. 2011 29:1005-1010; all of which is incorporated herein in their entirety), the present disclosure describes their formulation and use in delivering recombinant SS18::SSX fusion peptide epitopes and at least one cancer-testis antigen (CTA) epitope, or nucleic acids (e.g., mRNA, cDNA) encoding the same, contained therein.

Complexes, micelles, liposomes or particles can be prepared containing these lipidoids and therefore, can result in an effective delivery of the polynucleotide, as judged by the production of an encoded protein, following the injection of a lipidoid formulation via localized and/or systemic routes of administration. Lipidoid complexes of polynucleotides can be administered by various means including, but not limited to, intravenous, intramuscular, or subcutaneous routes.

In vivo delivery of nucleic acids may be affected by many parameters, including, but not limited to, the formulation composition, nature of particle PEGylation, degree of loading, polynucleotide to lipid ratio, and biophysical parameters such as, but not limited to, particle size (Akinc et al., Mol Ther. 2009 17:872-879; herein incorporated by reference in its entirety). As an example, small changes in the anchor chain length of poly(ethylene glycol) (PEG) lipids may result in significant effects on in vivo efficacy. Formulations with the different lipidoids, including, but not limited to penta[3-(1-laurylaminopropionyl)]-triethylenetetramine hydrochloride (TETA-5LAP; aka 98N12-5, see Murugaiah et al., Analytical Biochemistry, 401:61 (2010); herein incorporated by reference in its entirety), C12-200 (including derivatives and variants), and MD1, can be tested for in vivo activity.

The lipidoid referred to herein as “98N12-5” is disclosed by Akinc et al., Mol Ther. 2009 17:872-879 and is incorporated by reference in its entirety.

The lipidoid referred to herein as “C12-200” is disclosed by Love et al., Proc Natl Acad Sci USA. 2010 107:1864-1869 and Liu and Huang, Molecular Therapy. 2010 669-670; both of which are herein incorporated by reference in their entirety. The lipidoid formulations can include particles comprising either 3 or 4 or more components in addition to polynucleotides or peptides. As an example, formulations with certain lipidoids, include, but are not limited to, 98N12-5 and may contain 42% lipidoid, 48% cholesterol and 10% PEG (C14 alkyl chain length). As another example, formulations with certain lipidoids, include, but are not limited to, C12-200 and may contain 50% lipidoid, 10% disteroylphosphatidyl choline, 38.5% cholesterol, and 1.5% PEG-DMG.

In one embodiment, a polynucleotide formulated with a lipidoid for systemic intravenous administration can target the liver. For example, a final optimized intravenous formulation using polynucleotides, and comprising a lipid molar composition of 42% 98N12-5, 48% cholesterol, and 10% PEG-lipid with a final weight ratio of about 7.5 to 1 total lipid to polynucleotides, and a C14 alkyl chain length on the PEG lipid, with a mean particle size of roughly 50-60 nm, can result in the distribution of the formulation to be greater than 90% to the liver. (see, Akinc et al., Mol Ther. 2009 17:872-879; herein incorporated by reference in its entirety). In another example, an intravenous formulation using a C12-200 (see U.S. provisional application 61/175,770 and published international application WO2010129709, each of which is herein incorporated by reference in their entirety) lipidoid may have a molar ratio of 50/10/38.5/1.5 of C12-200/disteroylphosphatidyl choline/cholesterol/PEG-DMG, with a weight ratio of 7 to 1 total lipid to polynucleotides, and a mean particle size of 80 nm may be effective to deliver polynucleotides to hepatocytes (see, Love et al., Proc Natl Acad Sci USA. 2010 107:1864-1869 herein incorporated by reference in its entirety). In another embodiment, an MD1 lipidoid-containing formulation may be used to effectively deliver polynucleotides to hepatocytes in vivo.

The characteristics of optimized lipidoid formulations for intramuscular or subcutaneous routes may vary significantly depending on the target cell type and the ability of formulations to diffuse through the extracellular matrix into the blood stream. While a particle size of less than 150 nm may be desired for effective hepatocyte delivery due to the size of the endothelial fenestrae (see, Akinc et al., Mol Ther. 2009 17:872-879 herein incorporated by reference in its entirety), use of a lipidoid-formulated recombinant SS18::SSX fusion peptide epitopes and at least one cancer-testis antigen (CTA) epitope, or nucleic acids (e.g., mRNA, cDNA) encoding the same, to deliver the formulation to other cells types including, but not limited to, endothelial cells, myeloid cells, and muscle cells may not be similarly size-limited.

Use of lipidoid formulations to deliver siRNA in vivo to other non-hepatocyte cells such as myeloid cells and endothelium has been reported (see Akinc et al., Nat Biotechnol. 2008 26:561-569; Leuschner et al., Nat Biotechnol. 2011 29:1005-1010; Cho et al. Adv. Funct. Mater. 2009 19:3112-3118; 8.sup.th International Judah Folkman Conference, Cambridge, Mass. Oct. 8-9, 2010; each of which is herein incorporated by reference in its entirety). Effective delivery to myeloid cells, such as monocytes, lipidoid formulations may have a similar component molar ratio. Different ratios of lipidoids and other components including, but not limited to, disteroylphosphatidyl choline, cholesterol and PEG-DMG, may be used to optimize the formulation of the recombinant SS18::SSX fusion peptide epitopes and at least one cancer-testis antigen (CTA) epitope, or nucleic acids (e.g., mRNA, cDNA) encoding the same, for delivery to different cell types including, but not limited to, hepatocytes, myeloid cells, muscle cells, etc. For example, the component molar ratio may include, but is not limited to, 50% C12-200, 10% disteroylphosphatidyl choline, 38.5% cholesterol, and %1.5 PEG-DMG (see Leuschner et al., Nat Biotechnol 2011 29:1005-1010; herein incorporated by reference in its entirety). The use of lipidoid formulations for the localized delivery of nucleic acids or peptides to cells (such as, but not limited to, adipose cells and muscle cells) via either subcutaneous or intramuscular delivery, may not require all of the formulation components desired for systemic delivery, and as such may comprise only the lipidoid and the recombinant SS18::SSX fusion peptide epitopes and at least one cancer-testis antigen (CTA) epitope, or nucleic acids (e.g., mRNA, cDNA) encoding the same.

Combinations of different lipidoids may be used to improve the efficacy of polynucleotide directed protein production as the lipidoids may be able to increase cell transfection by the nucleic acid encoding the rFSP; and/or increase the translation of encoded protein (see Whitehead et al., Mol. Ther. 2011, 19:1688-1694, herein incorporated by reference in its entirety).

### Liposomes, Lipoplexes, and Lipid Nanoparticles

The recombinant SS18::SSX fusion peptide epitopes and at least one cancer-testis antigen (CTA) epitope, or nucleic acids (e.g., mRNA, cDNA) encoding the same, of the present disclosure can be formulated using one or more liposomes, lipoplexes, or lipid nanoparticles. In one embodiment, pharmaceutical compositions of recombinant SS18::SSX fusion peptide epitopes and at least one cancer-testis antigen (CTA) epitope, or nucleic acids (e.g., mRNA, cDNA) encoding the same, include liposomes. Liposomes are artificially-prepared vesicles which may primarily be composed of a lipid bilayer and may be used as a delivery vehicle for the administration of nutrients and pharmaceutical formulations. Liposomes can be of different sizes such as, but not limited to, a multilamellar vesicle (MLV) which may be hundreds of nanometers in diameter and may contain a series of concentric bilayers separated by narrow aqueous compartments, a small unicellular vesicle (SUV) which may be smaller than 50 nm in diameter, and a large unilamellar vesicle (LUV) which may be between 50 and 500 nm in diameter. Liposome design may include, but is not limited to, opsonins or ligands in order to improve the attachment of liposomes to unhealthy tissue or to activate events such as, but not limited to, endocytosis. Liposomes may contain a low or a high pH in order to improve the delivery of the pharmaceutical formulations.

The formation of liposomes may depend on the physicochemical characteristics such as, but not limited to, the pharmaceutical formulation entrapped and the liposomal ingredients, the nature of the medium in which the lipid vesicles are dispersed, the effective concentration of the entrapped substance and its potential toxicity, any additional processes involved during the application and/or delivery of the vesicles, the optimization size, polydispersity and the shelf-life of the vesicles for the intended application, and the batch-to-batch reproducibility and possibility of large-scale production of safe and efficient liposomal products.

As a non-limiting example, liposomes such as synthetic membrane vesicles may be prepared by the methods, apparatus and devices described in US Patent Publication No. US20130177638, US20130177637, US20130177636, US20130177635, US20130177634, US20130177633, US20130183375, US20130183373 and US20130183372, the contents of each of which are herein incorporated by reference in its entirety.

In one embodiment, pharmaceutical compositions described herein may include, without limitation, liposomes such as those formed from 1,2-dioleyloxy-N,N-dimethylaminopropane (DODMA) liposomes, DiLa2 liposomes from Marina Biotech (Bothell, Wash.), 1,2-dilinoleyloxy-3-dimethylaminopropane (DLin-DMA), 2,2-dilinoleyl-4-(2-dimethylaminoethyl)-[1,3]-dioxolane (DLin-KC2-DMA), and MC3 (US20100324120; herein incorporated by reference in its entirety) and liposomes which may deliver small molecule drugs such as, but not limited to, DOXIL® from Janssen Biotech, Inc. (Horsham, Pa.).

In one embodiment, pharmaceutical compositions described herein may include, without limitation, liposomes such as those formed from the synthesis of stabilized plasmid-lipid particles (SPLP) or stabilized nucleic acid lipid particle (SNALP) that have been previously described and shown to be suitable for oligonucleotide delivery in vitro and in vivo (see Wheeler et al. Gene Therapy. 1999 6:271-281; Zhang et al. Gene Therapy. 1999 6:1438-1447; Jeffs et al. Pharm Res. 2005 22:362-372; Morrissey et al., Nat Biotechnol. 2005 2:1002-1007; Zimmermann et al., Nature. 2006 441:111-114; Heyes et al. J Contr Rel. 2005 107:276-287; Semple et al. Nature Biotech. 2010 28:172-176; Judge et al. J Clin Invest. 2009 119:661-673; deFougerolles Hum Gene Ther. 2008 19:125-132; U.S. Patent Publication No US20130122104; all of which are incorporated herein in their entireties). The original manufacture method by Wheeler et al. was a detergent dialysis method, which was later improved by Jeffs et al. and is referred to as the spontaneous vesicle formation method. The liposome formulations are composed of 3 to 4 lipid components in addition to the polynucleotide. As an example a liposome can contain, but is not limited to, 55% cholesterol, 20% distcroylphosphatidyl choline (DSPC), 10% PEG-S-DSG, and 15% 1,2-dioleyloxy-N,N-dimethylaminopropane (DODMA), as described by Jeffs et al. As another example, certain liposome formulations may contain, but are not limited to, 48% cholesterol, 20% DSPC, 2% PEG-c-DMA, and 30% cationic lipid, where the cationic lipid can be 1,2-distearloxy-N,N-dimethylaminopropane (DSDMA), DODMA, DLin-DMA, or 1,2-dilinolenyloxy-3-dimethylaminopropane (DLenDMA), as described by Heyes et al.

In some embodiments, liposome formulations may comprise from about 25.0% cholesterol to about 40.0% cholesterol, from about 30.0% cholesterol to about 45.0% cholesterol, from about 35.0% cholesterol to about 50.0% cholesterol and/or from about 48.5% cholesterol to about 60% cholesterol. In a preferred embodiment, formulations may comprise a percentage of cholesterol selected from the group consisting of 28.5%, 31.5%, 33.5%, 36.5%, 37.0%, 38.5%, 39.0% and 43.5%. In some embodiments, formulations may comprise from about 5.0% to about 10.0% DSPC and/or from about 7.0% to about 15.0% DSPC.

In one embodiment, pharmaceutical compositions may include liposomes which may be formed to deliver recombinant SS18::SSX fusion peptide epitopes and at least one cancer-testis antigen (CTA) epitope, or nucleic acids (e.g., mRNA, cDNA) encoding the same, of the present technology. The recombinant SS18::SSX fusion peptide epitopes and at least one cancer-testis antigen (CTA) epitope, or nucleic acids (e.g., mRNA, cDNA) encoding the same, may be encapsulated by the liposome and/or it may be contained in an aqueous core which may then be encapsulated by the liposome (see International Pub. Nos. WO2012031046, WO2012031043, WO2012030901 and WO2012006378 and US Patent Publication No. US20130189351, US20130195969 and US20130202684; the contents of each of which are herein incorporated by reference in their entirety).

In another embodiment, liposomes may be formulated for targeted delivery. As a non-limiting example, the liposome may be formulated for targeted delivery to the liver. The liposome used for targeted delivery may include, but is not limited to, the liposomes described in and methods of making liposomes described in US Patent Publication No. US20130195967, the contents of which are herein incorporated by reference in its entirety.

In another embodiment, the recombinant SS18::SSX fusion peptide epitopes and at least one cancer-testis antigen (CTA) epitope, or nucleic acids (e.g., mRNA, cDNA) encoding the same, may be formulated in a cationic oil-in-water emulsion where the emulsion particle comprises an oil core and a cationic lipid which can interact with the recombinant SS18::SSX fusion peptide epitopes and at least one cancer-testis antigen (CTA) epitope, or nucleic acids (e.g., mRNA, cDNA) encoding the same, anchoring the molecule to the emulsion particle (see International Pub. No. WO2012006380; herein incorporated by reference in its entirety).

In one embodiment, the recombinant SS18::SSX fusion peptide epitopes and at least one cancer-testis antigen (CTA) epitope, or nucleic acids (e.g., mRNA, cDNA) encoding the same, may be formulated in a water-in-oil emulsion comprising a continuous hydrophobic phase in which the hydrophilic phase is dispersed. As a non-limiting example, the emulsion may be made by the methods described in International Publication No. WO201087791, the contents of which are herein incorporated by reference in its entirety.

In another embodiment, the lipid formulation may include at least cationic lipid, a lipid which may enhance transfection and a least one lipid which contains a hydrophilic head group linked to a lipid moiety (International Pub. No. WO2011076807 and U.S. Pub. No. 20110200582; the contents of each of which is herein incorporated by reference in their entirety). In another embodiment, the recombinant SS18::SSX fusion peptide epitopes and at least one cancer-testis antigen (CTA) epitope, or nucleic acids (e.g., mRNA, cDNA) encoding the same, may be formulated in a lipid vesicle which may have crosslinks between functionalized lipid bilayers (see U.S. Pub. No. 20120177724, the contents of which is herein incorporated by reference in its entirety).

In one embodiment, the recombinant SS18::SSX fusion peptide epitopes and at least one cancer-testis antigen (CTA) epitope, or nucleic acids (e.g., mRNA, cDNA) encoding the same, may be formulated in a liposome as described in International Patent Publication No. WO2013086526, the contents of which is herein incorporated by reference in its entirety. The recombinant SS18::SSX fusion peptide epitopes and at least one cancer-testis antigen (CTA) epitope, or nucleic acids (e.g., mRNA, cDNA) encoding the same, may be encapsulated in a liposome using reverse pH gradients and/or optimized internal buffer compositions as described in International Patent Publication No. WO2013086526, the contents of which is herein incorporated by reference in its entirety.

In one embodiment, the recombinant SS18::SSX fusion peptide epitopes and at least one cancer-testis antigen (CTA) epitope, or nucleic acids (e.g., mRNA, cDNA) encoding the same, pharmaceutical compositions may be formulated in liposomes such as, but not limited to, DiLa2 liposomes (Marina Biotech, Bothell, Wash.), SMARTICLES® (Marina Biotech, Bothell, Wash.), neutral DOPC (1,2-dioleoyl-sn-glycero-3-phosphocholine) based liposomes (e.g., siRNA delivery for ovarian cancer (Landen et al. Cancer Biology & Therapy 2006 5(12)1708-1713); herein incorporated by reference in its entirety) and hyaluronan-coated liposomes (Quiet Therapeutics, Israel).

In one embodiment, the cationic lipid may be a low molecular weight cationic lipid such as those described in US Patent Application No. 20130090372, the contents of which are herein incorporated by reference in its entirety.

In one embodiment, the recombinant SS18::SSX fusion peptide epitopes and at least one cancer-testis antigen (CTA) epitope, or nucleic acids (e.g., mRNA, cDNA) encoding the same, may be formulated in a lipid vesicle which may have crosslinks between functionalized lipid bilayers.

In one embodiment, the recombinant SS18::SSX fusion peptide epitopes and at least one cancer-testis antigen (CTA) epitope, or nucleic acids (e.g., mRNA, cDNA) encoding the same, may be formulated in a liposome comprising a cationic lipid. The liposome may have a molar ratio of nitrogen atoms in the cationic lipid to the phosphates in the RNA (N:P ratio) of between 1:1 and 20:1 as described in International Publication No. WO2013006825, herein incorporated by reference in its entirety. In another embodiment, the liposome may have a N:P ratio of greater than 20:1 or less than 1:1.

In one embodiment, the recombinant SS18::SSX fusion peptide epitopes and at least one cancer-testis antigen (CTA) epitope, or nucleic acids (e.g., mRNA, cDNA) encoding the same, may be formulated in a lipid-polycation complex. The formation of the lipid-polycation complex may be accomplished by methods known in the art and/or as described in U.S. Pub. No. 20120178702, herein incorporated by reference in its entirety. As a non-limiting example, the polycation may include a cationic peptide or a polypeptide such as, but not limited to, polylysine, polyornithine and/or polyarginine and the cationic peptides described in International Pub. No. WO2012013326 or US Patent Pub. No. US20130142818; each of which is herein incorporated by reference in its entirety. In another embodiment, the recombinant SS18::SSX fusion peptide epitopes and at least one cancer-testis antigen (CTA) epitope, or nucleic acids (e.g., mRNA, cDNA) encoding the same, may be formulated in a lipid-polycation complex which may further include a non-cationic lipid such as, but not limited to, cholesterol or dioleoyl phosphatidylethanolamine (DOPE).

In one embodiment, the recombinant SS18::SSX fusion peptide epitopes and at least one cancer-testis antigen (CTA) epitope, or nucleic acids (e.g., mRNA, cDNA) encoding the same, may be formulated in an aminoalcohol lipidoid. Aminoalcohol lipidoids which may be used in the present disclosure may be prepared by the methods described in U.S. Pat. No. 8,450,298, herein incorporated by reference in its entirety.

The liposome formulation may be influenced by, but not limited to, the selection of the cationic lipid component, the degree of cationic lipid saturation, the nature of the PEGylation, ratio of all components and biophysical parameters such as size. In one example by Semple et al. (Semple et al. Nature Biotech. 2010 28:172-176; herein incorporated by reference in its entirety), the liposome formulation was composed of 57.1% cationic lipid, 7.1% dipalmitoylphosphatidylcholine, 34.3% cholesterol, and 1.4% PEG-c-DMA. As another example, changing the composition of the cationic lipid could more effectively deliver siRNA to various antigen presenting cells (Basha et al. Mol Ther. 2011 19:2186-2200; herein incorporated by reference in its entirety). In some embodiments, liposome formulations may comprise from about 35 to about 45% cationic lipid, from about 40% to about 50% cationic lipid, from about 50% to about 60% cationic lipid and/or from about 55% to about 65% cationic lipid. In some embodiments, the ratio of lipid to mRNA in liposomes may be from about 5:1 to about 20:1, from about 10:1 to about 25:1, from about 15:1 to about 30:1 and/or at least 30:1.

In some embodiments, the ratio of PEG in the lipid nanoparticle (LNP) formulations may be increased or decreased and/or the carbon chain length of the PEG lipid may be modified from C14 to C18 to alter the pharmacokinetics and/or biodistribution of the LNP formulations. As a non-limiting example, LNP formulations may contain from about 0.5% to about 3.0%, from about 1.0% to about 3.5%, from about 1.5% to about 4.0%, from about 2.0% to about 4.5%, from about 2.5% to about 5.0% and/or from about 3.0% to about 6.0% of the lipid molar ratio of PEG-c-DOMG (R-3-[(ω-methoxy-poly(ethyleneglycol)2000)carbamoyl)]-1,2-dimyristyloxypropyl-3-amine) (also referred to herein as PEG-DOMG) as compared to the cationic lipid, DSPC and cholesterol. In another embodiment the PEG-c-DOMG may be replaced with a PEG lipid such as, but not limited to, PEG-DSG (1,2-Distearoyl-sn-glycerol, methoxypolyethylene glycol), PEG-DMG (1,2-Dimyristoyl-sn-glycerol) and/or PEG-DPG (1,2-Dipalmitoyl-sn-glycerol, methoxypolyethylene glycol). The cationic lipid may be selected from any lipid known in the art such as, but not limited to, DLin-MC3-DMA, DLin-DMA, C12-200 and DLin-KC2-DMA.

In one embodiment, the recombinant SS18::SSX fusion peptide epitopes and at least one cancer-testis antigen (CTA) epitope, or nucleic acids (e.g., mRNA, cDNA) encoding the same, may be formulated in a lipid nanoparticle such as those described in International Publication No. WO2012170930, the contents of which is herein incorporated by reference in its entirety.

In one embodiment, the recombinant SS18::SSX fusion peptide epitopes and at least one cancer-testis antigen (CTA) epitope, or nucleic acids (e.g., mRNA, cDNA) encoding the same, formulation is a nanoparticle which may comprise at least one lipid. The lipid may be selected from, but is not limited to, DLin-DMA, DLin-K-DMA, 98N12-5, C12-200, DLin-MC3-DMA, DLin-KC2-DMA, DODMA, PLGA, PEG, PEG-DMG, PEGylated lipids and amino alcohol lipids. In another aspect, the lipid may be a cationic lipid such as, but not limited to, DLin-DMA, DLin-D-DMA, DLin-MC3-DMA, DLin-KC2-DMA, DODMA and amino alcohol lipids. The amino alcohol cationic lipid may be the lipids described in and/or made by the methods described in US Patent Publication No. US20130150625, herein incorporated by reference in its entirety. As a non-limiting example, the cationic lipid may be 2-amino-3-[(9Z,12Z)-octadeca-9,12-dien-1-yloxy]-2-{[(9Z,2Z)-octadeca-9,12-dien-1-yloxy]methyl}propan-1-ol (Compound 1 in US20130150625); 2-amino-3-[(9Z)-octadec-9-en-1-yloxy]-2-{[(9Z)-octadec-9-en-1-yloxy]methyl}propan-1-ol (Compound 2 in US20130150625); 2-amino-3-[(9Z,12Z)-octadeca-9,12-dien-1-yloxy]-2-[(octyloxy)methyl]propan-1-ol (Compound 3 in US20130150625); and 2-(dimethylamino)-3-[(9Z,12Z)-octadeca-9,12-dien-1-yloxy]-2-{[(9Z,12Z)-octadeca-9,12-dien-1-yloxy]methyl}propan-1-ol (Compound 4 in US20130150625); or any pharmaceutically acceptable salt or stereoisomer thereof.

Lipid nanoparticle formulations typically comprise a lipid, in particular, an ionizable cationic lipid, for example, 2,2-dilinoleyl-4-dimethylaminoethyl-[1,3]-dioxolane (DLin-KC2-DMA), dilinoleyl-methyl-4-dimethylaminobutyrate (DLin-MC3-DMA), or di((Z)-non-2-en-1-yl) 9-((4-(dimethylamino)butanoyl)oxy)heptadecanedioate (L319), and further comprise a neutral lipid, a sterol and a molecule capable of reducing particle aggregation, for example a PEG or PEG-modified lipid.

In one embodiment, the lipid nanoparticle formulation consists essentially of (i) at least one lipid selected from the group consisting of 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319); (ii) a neutral lipid selected from DSPC, DPPC, POPC, DOPE and SM; (iii) a sterol, e.g., cholesterol; and (iv) a PEG-lipid, e.g., PEG-DMG or PEG-cDMA, in a molar ratio of about 20-60% cationic lipid:5-25% neutral lipid:25-55% sterol; 0.5-15% PEG-lipid.

In one embodiment, the formulation includes from about 25% to about 75% on a molar basis of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), e.g., from about 35 to about 65%, from about 45 to about 65%, about 60%, about 57.5%, about 50% or about 40% on a molar basis.

In one embodiment, the formulation includes from about 0.5% to about 15% on a molar basis of the neutral lipid e.g., from about 3 to about 12%, from about 5 to about 10% or about 15%, about 10%, or about 7.5% on a molar basis. Exemplary neutral lipids include, but are not limited to, DSPC, POPC, DPPC, DOPE and SM. In one embodiment, the formulation includes from about 5% to about 50% on a molar basis of the sterol (e.g., about 15 to about 45%, about 20 to about 40%, about 40%, about 38.5%, about 35%, or about 31% on a molar basis. An exemplary sterol is cholesterol. In one embodiment, the formulation includes from about 0.5% to about 20% on a molar basis of the PEG or PEG-modified lipid (e.g., about 0.5 to about 10%, about 0.5 to about 5%, about 1.5%, about 0.5%, about 1.5%, about 3.5%, or about 5% on a molar basis. In one embodiment, the PEG or PEG modified lipid comprises a PEG molecule of an average molecular weight of 2,000 Da. In other embodiments, the PEG or PEG modified lipid comprises a PEG molecule of an average molecular weight of less than 2,000, for example around 1,500 Da, around 1,000 Da, or around 500 Da. Exemplary PEG-modified lipids include, but are not limited to, PEG-distearoyl glycerol (PEG-DMG) (also referred herein as PEG-C14 or C14-PEG), PEG-cDMA (further discussed in Reyes et al. J. Controlled Release, 107, 276-287 (2005) the contents of which are herein incorporated by reference in its entirety)

In one embodiment, the formulations of the present disclosure include 25-75% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), 0.5-15% of the neutral lipid, 5-50% of the sterol, and 0.5-20% of the PEG or PEG-modified lipid on a molar basis.

In one embodiment, the formulations of the present disclosure include 35-65% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), 3-12% of the neutral lipid, 15-45% of the sterol, and 0.5-10% of the PEG or PEG-modified lipid on a molar basis.

In one embodiment, the formulations of the present disclosure include 45-65% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), 5-10% of the neutral lipid, 25-40% of the sterol, and 0.5-10% of the PEG or PEG-modified lipid on a molar basis.

In one embodiment, the formulations of the present disclosure include about 60% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), about 7.5% of the neutral lipid, about 31% of the sterol, and about 1.5% of the PEG or PEG-modified lipid on a molar basis.

In one embodiment, the formulations of the present disclosure include about 50% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), about 10% of the neutral lipid, about 38.5% of the sterol, and about 1.5% of the PEG or PEG-modified lipid on a molar basis.

In one embodiment, the formulations of the present disclosure include about 50% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), about 10% of the neutral lipid, about 35% of the sterol, about 4.5% or about 5% of the PEG or PEG-modified lipid, and about 0.5% of the targeting lipid on a molar basis.

In one embodiment, the formulations of the present disclosure include about 40% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), about 15% of the neutral lipid, about 40% of the sterol, and about 5% of the PEG or PEG-modified lipid on a molar basis.

In one embodiment, the formulations of the present disclosure include about 57.2% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), about 7.1% of the neutral lipid, about 34.3% of the sterol, and about 1.4% of the PEG or PEG-modified lipid on a molar basis.

In one embodiment, the formulations of the present disclosure include about 57.5% of a cationic lipid selected from the PEG lipid is PEG-cDMA (PEG-cDMA is further discussed in Reyes et al. (J. Controlled Release, 107, 276-287 (2005), the contents of which are herein incorporated by reference in its entirety), about 7.5% of the neutral lipid, about 31.5% of the sterol, and about 3.5% of the PEG or PEG-modified lipid on a molar basis.

In preferred embodiments, lipid nanoparticle formulation consists essentially of a lipid mixture in molar ratios of about 20-70% cationic lipid:5-45% neutral lipid:20-55% cholesterol:0.5-15% PEG-modified lipid; more preferably in a molar ratio of about 20-60% cationic lipid:5-25% neutral lipid:25-55% cholesterol:0.5-15% PEG-modified lipid.

In particular embodiments, the molar lipid ratio is approximately 50/10/38.5/1.5 (mol % cationic lipid/neutral lipid, e.g., DSPC/Chol/PEG-modified lipid, e.g., PEG-DMG, PEG-DSG or PEG-DPG), 57.2/7.1134.3/1.4 (mol % cationic lipid/neutral lipid, e.g., DPPC/Chol/PEG-modified lipid, e.g., PEG-cDMA), 40/15/40/5 (mol % cationic lipid/neutral lipid, e.g., DSPC/Chol/PEG-modified lipid, e.g., PEG-DMG), 50/10/35/4.5/0.5 (mol % cationic lipid/neutral lipid, e.g., DSPC/Chol/PEG-modified lipid, e.g., PEG-DSG), 50/10/35/5 (cationic lipid/neutral lipid, e.g., DSPC/Chol/PEG-modified lipid, e.g., PEG-DMG), 40/10/40/10 (mol % cationic lipid/neutral lipid, e.g., DSPC/Chol/PEG-modified lipid, e.g., PEG-DMG or PEG-cDMA), 35/15/40/10 (mol % cationic lipid/neutral lipid, e.g., DSPC/Chol/PEG-modified lipid, e.g., PEG-DMG or PEG-cDMA) or 52/13/30/5 (mol % cationic lipid/neutral lipid, e.g., DSPC/Chol/PEG-modified lipid, e.g., PEG-DMG or PEG-cDMA).

Exemplary lipid nanoparticle compositions and methods of making same are described, for example, in Semple et al. (2010) Nat. Biotechnol. 28:172-176; Jayarama et al. (2012), Angew. Chem. Int. Ed., 51: 8529-8533; and Maier et al. (2013) Molecular Therapy 21, 1570-1578 (the contents of each of which are incorporated herein by reference in their entirety).

In one embodiment, the lipid nanoparticle formulations described herein may comprise a cationic lipid, a PEG lipid and a structural lipid and optionally comprise a non-cationic lipid. As a non-limiting example, the lipid nanoparticle may comprise about 40-60% of cationic lipid, about 5-15% of a non-cationic lipid, about 1-2% of a PEG lipid and about 30-50% of a structural lipid. As another non-limiting example, the lipid nanoparticle may comprise about 50% cationic lipid, about 10% non-cationic lipid, about 1.5% PEG lipid and about 38.5% structural lipid. As yet another non-limiting example, the lipid nanoparticle may comprise about 55% cationic lipid, about 10% non-cationic lipid, about 2.5% PEG lipid and about 32.5% structural lipid. In one embodiment, the cationic lipid may be any cationic lipid described herein such as, but not limited to, DLin-KC2-DMA, DLin-MC3-DMA and L319.

In one embodiment, the lipid nanoparticle formulations described herein may be 4 component lipid nanoparticles. The lipid nanoparticle may comprise a cationic lipid, a non-cationic lipid, a PEG lipid and a structural lipid. As a non-limiting example, the lipid nanoparticle may comprise about 40-60% of cationic lipid, about 5-15% of a non-cationic lipid, about 1-2% of a PEG lipid and about 30-50% of a structural lipid. As another non-limiting example, the lipid nanoparticle may comprise about 50% cationic lipid, about 10% non-cationic lipid, about 1.5% PEG lipid and about 38.5% structural lipid. As yet another non-limiting example, the lipid nanoparticle may comprise about 55% cationic lipid, about 10% non-cationic lipid, about 2.5% PEG lipid and about 32.5% structural lipid. In one embodiment, the cationic lipid may be any cationic lipid described herein such as, but not limited to, DLin-KC2-DMA, DLin-MC3-DMA and L319.

In one embodiment, the lipid nanoparticle formulations described herein may comprise a cationic lipid, a non-cationic lipid, a PEG lipid and a structural lipid. As a non-limiting example, the lipid nanoparticle comprise about 50% of the cationic lipid DLin-KC2-DMA, about 10% of the non-cationic lipid DSPC, about 1.5% of the PEG lipid PEG-DOMG and about 38.5% of the structural lipid cholesterol. As a non-limiting example, the lipid nanoparticle comprise about 50% of the cationic lipid DLin-MC3-DMA, about 10% of the non-cationic lipid DSPC, about 1.5% of the PEG lipid PEG-DOMG and about 38.5% of the structural lipid cholesterol. As a non-limiting example, the lipid nanoparticle comprise about 50% of the cationic lipid DLin-MC3-DMA, about 10% of the non-cationic lipid DSPC, about 1.5% of the PEG lipid PEG-DMG and about 38.5% of the structural lipid cholesterol. As yet another non-limiting example, the lipid nanoparticle comprise about 55% of the cationic lipid L319, about 10% of the non-cationic lipid DSPC, about 2.5% of the PEG lipid PEG-DMG and about 32.5% of the structural lipid cholesterol.

In one embodiment, the cationic lipid may be selected from, but not limited to, a cationic lipid described in International Publication Nos. WO2012040184, WO2011153120, WO2011149733, WO2011090965, WO2011043913, WO2011022460, WO2012061259, WO2012054365, WO2012044638, WO2010080724, WO201021865, WO2008103276, WO2013086373 and WO2013086354, U.S. Pat. Nos. 7,893,302, 7,404,969, 8,283,333, and 8,466,122 and US Patent Publication No. US20100036115, US20120202871, US20130064894, US20130129785, US20130150625, US20130178541 and US20130225836; the contents of each of which are herein incorporated by reference in their entirety. In another embodiment, the cationic lipid may be selected from, but not limited to, formula A described in International Publication Nos. WO2012040184, WO2011153120, WO2011149733, WO2011090965, WO2011043913, WO2011022460, WO2012061259, WO2012054365, WO2012044638 and WO2013116126 or US Patent Publication No. US20130178541 and US20130225836; the contents of each of which is herein incorporated by reference in their entirety. In yet another embodiment, the cationic lipid may be selected from, but not limited to, formula CLI-CLXXIX of International Publication No. WO2008103276, formula CLI-CLXXIX of U.S. Pat. No. 7,893,302, formula CLI-CLXXXXII of U.S. Pat. No. 7,404,969 and formula I-VI of US Patent Publication No. US20100036115, formula I of US Patent Publication No US20130123338; each of which is herein incorporated by reference in their entirety. As a non-limiting example, the cationic lipid may be selected from (20Z,23Z)—N,N-dimethylnonacosa-20,23-dien-10-amine, (17Z,20Z) N,N-dimemylhexacosa-17,20-dien-9-amine, (1Z,19Z) N5N-dimethylpentacosa-16, 19-dien-8-amine, (13Z,16Z)—N,N-dimethyldocosa-13,16-dien-5-amine, (12Z,15Z)—N,N-dimethylhenicosa-12,15-dien-4-amine, (14Z,17Z)—N,N-dimethyltricosa-14,17-diLen-6-amine, (15Z,18Z)—N,N-dimethyltetracosa-15,18-dien-7-amine, (18Z,21 Z)—N,N-dimethylheptacosa-18,21-dien-10-amine, (15Z,18Z)—N,N-dimethyltetracosa-15,18-dien-5-amine, (14Z,17Z)—N,N-dimethyltricosa-14,17-dien-4-amine, (19Z,22Z) N,N-dimeihyloctacosa-19,22-dien-9-amine, (18Z,21 Z) N,N-dimethylheptacosa-18,21-dien-8-amine, (17Z,20Z)—N,N-dimethylhexacosa-17,20-dien-7-amine, (16Z,19Z)—N,N-dimethylpentacosa-16,19-dien-6-amine, (22Z,25Z)—N,N-dimethylhentriaconta-22,25-dien-10-amine, (21 Z,24Z)—N,N-dimethyltriaconta-21,24-dien-9-amine, (18Z)—N,N-dimetylheptacos-18-en-10-amine, (17Z)—N,N-dimethylhexacos-17-en-9-amine, (19Z,22Z)—N,N-dimethyloctacosa-19,22-dien-7-amine, N,N-dimethylheptacosan-10-amine, (20Z,23Z)—N-ethyl-N-methylnonacosa-20,23-dien-10-amine, 1-[(11Z,14Z)-1-nonylicosa-11,14-dien-1-yl]pyrrolidine, (20Z) N,N-dimethylheptacos-20-en-10-amine, (15Z)—N,N-dimethyleptacos-15-en-10-amine, (14Z)—N,N-dimethylnonacos-14-en-10-amine, (17Z)—N,N-dimethylnonacos-17-en-10-amine, (24Z)—N,N-dimethyltritriacont-24-en-10-amine, (20Z)—N,N-dimethylnonacos-20-en-10-amine, (22Z)—N,N-dimethylhentriacont-22-en-10-amine, (16Z)—N,N-dimethylpentacos-16-en-8-amine, (12Z,15Z)—N,N-dimethyl-2-nonylhenicosa-12,15-dien-1-amine, (13Z,16Z)—N,N-dimethyl-3-nonyldocosa-13,16-dien-1-amine, N,N-dimethyl-1-[(1S,2R)-2-octylcyclopropyl]eptadecan-8-amine, 1-[(1S,2R)-2-hexylcyclopropyl]-N,N-dimethylnonadecan-10-amine, N,N-dimethyl-1-[(1S,2R)-2-octylcyclopropyl]nonadecan-10-amine, N,N-dimethyl-21-[(1S,2R)-2-octylcyclopropyl]henicosan-10-amine,N,N-dimethyl-1-[(1S,2S)-2-{[(1R,2R)-2-pentylcyclopropyl]methyl}cyclopropyl]nonadecan-10-amine,N,N-dimethyl-1-[(1S,2R)-2-octylcyclopropyl]hexadecan-8-amine, N,N-dimethyl-[(1R,2S)-2-undecylcyclopropyl]tetradecan-5-amine, N,N-dimethyl-3-{7-[(1S,2R)-2-octylcyclopropyl]heptyl}dodecan-1-amine, 1-[(1R,2S)-2-heptylcyclopropyl]-N,N-dimethyloctadecan-9-amine, 1-[(1S,2R)-2-decylcyclopropyl]-N,N-dimethylpentadecan-6-amine, N,N-dimethyl-1-[(1S,2R)-2-octylcyclopropyl]pentadecan-8-amine, R—N,N-dimethyl-1-[(9Z,12Z)-octadeca-9,12-dien-1-yloxy]-3-(octyloxy)propan-2-amine, S N,N-dimethyl-1-[(9Z,12Z)-octadeca-9,12-dien-1-yloxy]-3-(octyloxy)propan-2-amine, 1-{2-[(9Z,12Z)-octadeca-9,12-dien-1-yloxy]-1-[(octyloxy)methyl]ethyl}pyrrolidine, (2S) N,N-dimethyl-1-[(9Z,12Z)-octadeca-9,12-dien-1-yloxy]-3-[(5Z)-oct-5-en-1-yloxy]propan-2-amine, 1-{2-[(9Z,12Z)-octadeca-9,12-dien-1-yloxy]-1-[(octyloxy)methyl]ethyl}azetidine, (2S)-1-(hexyloxy)-N,N-dimethyl-3-[(9Z,12Z)-octadeca-9,12-dien-1-yloxy]propan-2-amine, (2S)-1-(heptyloxy)-N,N-dimethyl-3-[(9Z,12Z)-octadeca-9,12-dien-1-yloxy]propan-2-amine, N,N-dimethyl-1-(nonyloxy)-3-[(9Z,12Z)-octadeca-9,12-dien-1-yloxy]propan-2-amine, N,N-dimethyl-1-[(9Z)-octadec-9-en-1-yloxy]-3-(octyloxy)propan-2-amine; (2S)—N,N-dimethyl-1-[(6Z,9Z,12Z)-octadeca-6,9,12-trien-1-yloxy]-3-(octyloxy)propan-2-amine, (2S)-1-[(11Z,14Z)-icosa-11,14-dien-1-yloxy]-N,N-dimethyl-3-(pentyloxy)propan-2-amine, (2S)-1-(hexyloxy)-3-[(11Z,14Z)-icosa-11,14-dien-1-yloxy]-N,N-dimethylpropan-2-amine, 1-[(11Z,14Z)-icosa-11,14-dien-1-yloxy]-N,N-dimethyl-3-(octyloxy)propan-2-amine, 1-[(13Z,16Z)-docosa-13,16-dien-1-yloxy]-N,N-dimethyl-3-(octyloxy)propan-2-amine, (2S)-1-[(13Z,16Z)-docosa-13,16-dien-1-yloxy]-3-(hexyloxy)-N,N-dimethylpropan-2-amine, (2S)-1-[(13Z)-docos-13-en-1-yloxy]-3-(hexyloxy)-N,N-dimethylpropan-2-amine, 1-[(13Z)-docos-13-en-1-yloxy]-N,N-dimethyl-3-(octyloxy)propan-2-amine, 1-[(9Z)-hexadec-9-en-1-yloxy]-N,N-dimethyl-3-(octyloxy)propan-2-amine, (2R)—N,N-dimethyl-H(1-metoyloctyl)oxy]-3-[(9Z,12Z)-octadeca-9,12-dien-1-yloxy]propan-2-amine, (2R)-1-[(3,7-dimethyloctyl)oxy]-N,N-dimethyl-3-[(9Z,12Z)-octadeca-9,12-dien-1-yloxy]propan-2-amine, N,N-dimethyl-1-(octyloxy)-3-({8-[(1S,2S)-2-{[(1R,2R)-2-pentylcyclopropyl]methyl}cyclopropyl]octyl}oxy)propan-2-amine, N,N-dimethyl-1-{[8-(2-oclylcyclopropyl)octyl]oxy}-3-(octyloxy)propan-2-amine and (11E,20Z,23Z) N,N-dimethylnonacosa-11,20,2-trien-10-amine or a pharmaceutically acceptable salt or stereoisomer thereof.

In one embodiment, the lipid may be a cleavable lipid such as those described in International Publication No. WO2012170889, herein incorporated by reference in its entirety.

In another embodiment, the lipid may be a cationic lipid such as, but not limited to, Formula (I) of U.S. Patent Application No. US20130064894, the contents of which are herein incorporated by reference in its entirety.

In one embodiment, the cationic lipid may be synthesized by methods known in the art and/or as described in International Publication Nos. WO2012040184, WO2011153120, WO2011149733, WO2011090965, WO2011043913, WO2011022460, WO2012061259, WO2012054365, WO2012044638, WO2010080724, WO201021865, WO2013086373 and WO2013086354; the contents of each of which are herein incorporated by reference in their entirety.

In another embodiment, the cationic lipid may be a trialkyl cationic lipid. Non-limiting examples of trialkyl cationic lipids and methods of making and using the trialkyl cationic lipids are described in International Patent Publication No. WO2013126803, the contents of which are herein incorporated by reference in its entirety.

In one embodiment, the LNP formulations of the recombinant SS18::SSX fusion peptide epitopes and at least one cancer-testis antigen (CTA) epitope, or nucleic acids (e.g., mRNA, cDNA) encoding the same, may contain PEG-c-DOMG at 3% lipid molar ratio. In another embodiment, the LNP formulations recombinant SS18::SSX fusion peptide epitopes and at least one cancer-testis antigen (CTA) epitope, or nucleic acids (e.g., mRNA, cDNA) encoding the same, may contain PEG-c-DOMG at 1.5% lipid molar ratio.

In one embodiment, the pharmaceutical compositions of the recombinant SS18::SSX fusion peptide epitopes and at least one cancer-testis antigen (CTA) epitope, or nucleic acids (e.g., mRNA, cDNA) encoding the same, may include at least one of the PEGylated lipids described in International Publication No. WO2012099755, the contents of which is herein incorporated by reference in its entirety.

In one embodiment, the LNP formulation may contain PEG-DMG 2000 (1,2-dimyristoyl-sn-glycero-3-phophoethanolamine-N-[methoxy(polyethylene glycol)-2000). In one embodiment, the LNP formulation may contain PEG-DMG 2000, a cationic lipid known in the art and at least one other component. In another embodiment, the LNP formulation may contain PEG-DMG 2000, a cationic lipid known in the art, DSPC and cholesterol. As a non-limiting example, the LNP formulation may contain PEG-DMG 2000, DLin-DMA, DSPC and cholesterol. As another non-limiting example the LNP formulation may contain PEG-DMG 2000, DLin-DMA, DSPC and cholesterol in a molar ratio of 2:40:10:48 (see e.g., Geall et al., Nonviral delivery of self-amplifying RNA vaccines, PNAS 2012; PMID: 22908294; herein incorporated by reference in its entirety).

In one embodiment, the LNP formulation may be formulated by the methods described in International Publication Nos. WO2011127255 or WO2008103276, the contents of each of which is herein incorporated by reference in their entirety. As a non-limiting example, the recombinant SS18::SSX fusion peptide epitopes and at least one cancer-testis antigen (CTA) epitope, or nucleic acids (e.g., mRNA, cDNA) encoding the same, described herein may be encapsulated in LNP formulations as described in WO2011127255 and/or WO2008103276; each of which is herein incorporated by reference in their entirety.

In one embodiment, the recombinant SS18::SSX fusion peptide epitopes and at least one cancer-testis antigen (CTA) epitope, or nucleic acids (e.g., mRNA, cDNA) encoding the same, described herein may be formulated in a nanoparticle to be delivered by a parenteral route as described in U.S. Pub. No. US20120207845; the contents of which are herein incorporated by reference in its entirety.

In one embodiment, the recombinant SS18::SSX fusion peptide epitopes and at least one cancer-testis antigen (CTA) epitope, or nucleic acids (e.g., mRNA, cDNA) encoding the same, may be formulated in a lipid nanoparticle made by the methods described in US Patent Publication No US20130156845 or International Publication No WO2013093648 or WO2012024526, each of which is herein incorporated by reference in its entirety.

The lipid nanoparticles described herein may be made in a sterile environment by the system and/or methods described in US Patent Publication No. US20130164400, herein incorporated by reference in its entirety.

In one embodiment, the LNP formulation may be formulated in a nanoparticle such as a nucleic acid-lipid particle described in U.S. Pat. No. 8,492,359, the contents of which are herein incorporated by reference in its entirety. As a non-limiting example, the lipid particle may comprise one or more active agents or therapeutic agents; one or more cationic lipids comprising from about 50 mol % to about 85 mol % of the total lipid present in the particle; one or more non-cationic lipids comprising from about 13 mol % to about 49.5 mol % of the total lipid present in the particle; and one or more conjugated lipids that inhibit aggregation of particles comprising from about 0.5 mol % to about 2 mol % of the total lipid present in the particle. The nucleic acid in the nanoparticle may be the polynucleotides described herein and/or are known in the art.

In one embodiment, the LNP formulation may be formulated by the methods described in International Publication Nos. WO2011127255 or WO2008103276, the contents of each of which are herein incorporated by reference in their entirety. As a non-limiting example, recombinant SS18::SSX fusion peptide epitopes and at least one cancer-testis antigen (CTA) epitope, or nucleic acids (e.g., mRNA, cDNA) encoding the same, described herein may be encapsulated in LNP formulations as described in WO2011127255 and/or WO2008103276; the contents of each of which are herein incorporated by reference in their entirety.

In one embodiment, LNP formulations described herein may comprise a polycationic composition. As a non-limiting example, the polycationic composition may be selected from formula 1-60 of US Patent Publication No. US20050222064; the content of which is herein incorporated by reference in its entirety. In another embodiment, the LNP formulations comprising a polycationic composition may be used for the delivery of the recombinant SS18::SSX fusion peptide epitopes and at least one cancer-testis antigen (CTA) epitope, or nucleic acids (e.g., mRNA, cDNA) encoding the same, described herein in vivo and/or in vitro.

In one embodiment, the LNP formulations described herein may additionally comprise a permeability enhancer molecule. Non-limiting permeability enhancer molecules are described in US Patent Publication No. US20050222064; the content of which is herein incorporated by reference in its entirety.

In one embodiment, the recombinant SS18::SSX fusion peptide epitopes and at least one cancer-testis antigen (CTA) epitope, or nucleic acids (e.g., mRNA, cDNA) encoding the same, pharmaceutical compositions may be formulated in liposomes such as, but not limited to, DiLa2 liposomes (Marina Biotech, Bothell, Wash.), SMARTICLES® (Marina Biotech, Bothell, Wash.), neutral DOPC (1,2-dioleoyl-sn-glycero-3-phosphocholine) based liposomes (e.g., siRNA delivery for ovarian cancer (Landen et al. Cancer Biology & Therapy 2006 5(12)1708-1713); herein incorporated by reference in its entirety) and hyaluronan-coated liposomes (Quiet Therapeutics, Israel).

In one embodiment, the recombinant SS18::SSX fusion peptide epitopes and at least one cancer-testis antigen (CTA) epitope, or nucleic acids (e.g., mRNA, cDNA) encoding the same, may be formulated in a lyophilized gel-phase liposomal composition as described in US Publication No. US2012060293, herein incorporated by reference in its entirety.

The nanoparticle formulations may comprise a phosphate conjugate. The phosphate conjugate may increase in vivo circulation times and/or increase the targeted delivery of the nanoparticle. Phosphate conjugates for use with the present disclosure may be made by the methods described in International Application No. WO2013033438 or US Patent Publication No. US20130196948, the contents of each of which are herein incorporated by reference in its entirety. As a non-limiting example, the phosphate conjugates may include a compound of any one of the formulas described in International Application No. WO2013033438, herein incorporated by reference in its entirety.

The nanoparticle formulation may comprise a polymer conjugate. The polymer conjugate may be a water soluble conjugate. The polymer conjugate may have a structure as described in U.S. Patent Application No. 20130059360, the contents of which are herein incorporated by reference in its entirety. In one aspect, polymer conjugates with the recombinant SS18::SSX fusion peptide epitopes and at least one cancer-testis antigen (CTA) epitope, or nucleic acids (e.g., mRNA, cDNA) encoding the same, of the present disclosure may be made using the methods and/or segmented polymeric reagents described in U.S. Patent Application No. 20130072709, herein incorporated by reference in its entirety. In another aspect, the polymer conjugate may have pendant side groups comprising ring moieties such as, but not limited to, the polymer conjugates described in US Patent Publication No. US20130196948, the contents of which is herein incorporated by reference in its entirety.

The nanoparticle formulations may comprise a conjugate to enhance the delivery of nanoparticles of the present disclosure in a subject. Further, the conjugate may inhibit phagocytic clearance of the nanoparticles in a subject. In one aspect, the conjugate may be a “self” peptide designed from the human membrane protein CD47 (e.g., the “self” particles described by Rodriguez et al (Science 2013 339, 971-975), herein incorporated by reference in its entirety). As shown by Rodriguez et al. the self peptides delayed macrophage-mediated clearance of nanoparticles which enhanced delivery of the nanoparticles. In another aspect, the conjugate may be the membrane protein CD47 (e.g., see Rodriguez et al. Science 2013 339, 971-975, herein incorporated by reference in its entirety). Rodriguez et al. showed that, similarly to “self” peptides, CD47 can increase the circulating particle ratio in a subject as compared to scrambled peptides and PEG coated nanoparticles.

In one embodiment, the recombinant SS18::SSX fusion peptide epitopes and at least one cancer-testis antigen (CTA) epitope, or nucleic acids (e.g., mRNA, cDNA) encoding the same, of the present disclosure are formulated in nanoparticles which comprise a conjugate to enhance the delivery of the nanoparticles of the present disclosure in a subject. The conjugate may be the CD47 membrane or the conjugate may be derived from the CD47 membrane protein, such as the “self” peptide described previously. In another aspect the nanoparticle may comprise PEG and a conjugate of CD47 or a derivative thereof. In yet another aspect, the nanoparticle may comprise both the “self” peptide described above and the membrane protein CD47.

In another aspect, a “self” peptide and/or CD47 protein may be conjugated to a virus-like particle or pseudovirion, as described herein for delivery of the recombinant SS18::SSX fusion peptide epitopes and at least one cancer-testis antigen (CTA) epitope, or nucleic acids (e.g., mRNA, cDNA) encoding the same, of the present disclosure.

In another embodiment, pharmaceutical compositions comprising the recombinant SS18::SSX fusion peptide epitopes and at least one cancer-testis antigen (CTA) epitope, or nucleic acids (e.g., mRNA, cDNA) encoding the same, of the present disclosure and a conjugate which may have a degradable linkage. Non-limiting examples of conjugates include an aromatic moiety comprising an ionizable hydrogen atom, a spacer moiety, and a water-soluble polymer. As a non-limiting example, pharmaceutical compositions comprising a conjugate with a degradable linkage and methods for delivering such pharmaceutical compositions are described in US Patent Publication No. US20130184443, the contents of which are herein incorporated by reference in its entirety.

The nanoparticle formulations may be a carbohydrate nanoparticle comprising a carbohydrate carrier and a recombinant SS18::SSX fusion peptide epitopes and at least one cancer-testis antigen (CTA) epitope, or nucleic acids (e.g., mRNA, cDNA) encoding the same. As a non-limiting example, the carbohydrate carrier may include, but is not limited to, an anhydride-modified phytoglycogen or glycogen-type material, phtoglycogen octenyl succinate, phytoglycogen beta-dextrin, anhydride-modified phytoglycogen beta-dextrin. (See e.g., International Publication No. WO2012109121; the contents of which are herein incorporated by reference in its entirety).

Nanoparticle formulations of the present disclosure may be coated with a surfactant or polymer in order to improve the delivery of the particle. In one embodiment, the nanoparticle may be coated with a hydrophilic coating such as, but not limited to, PEG coatings and/or coatings that have a neutral surface charge. The hydrophilic coatings may help to deliver nanoparticles with larger payloads such as, but not limited to, recombinant SS18::SSX fusion peptide epitopes and at least one cancer-testis antigen (CTA) epitope, or nucleic acids (e.g., mRNA, cDNA) encoding the same, within the central nervous system. As a non-limiting example nanoparticles comprising a hydrophilic coating and methods of making such nanoparticles are described in US Patent Publication No. US20130183244, the contents of which are herein incorporated by reference in its entirety.

In one embodiment, the lipid nanoparticles of the present disclosure may be hydrophilic polymer particles. Non-limiting examples of hydrophilic polymer particles and methods of making hydrophilic polymer particles are described in US Patent Publication No. US20130210991, the contents of which are herein incorporated by reference in its entirety.

In another embodiment, the lipid nanoparticles of the present disclosure may be hydrophobic polymer particles.

Lipid nanoparticle formulations may be improved by replacing the cationic lipid with a biodegradable cationic lipid which is known as a rapidly eliminated lipid nanoparticle (reLNP). Ionizable cationic lipids, such as, but not limited to, DLinDMA, DLin-KC2-DMA, and DLin-MC3-DMA, have been shown to accumulate in plasma and tissues over time and may be a potential source of toxicity. The rapid metabolism of the rapidly eliminated lipids can improve the tolerability and therapeutic index of the lipid nanoparticles by an order of magnitude from a 1 mg/kg dose to a 10 mg/kg dose in rat. Inclusion of an enzymatically degraded ester linkage can improve the degradation and metabolism profile of the cationic component, while still maintaining the activity of the reLNP formulation. The ester linkage can be internally located within the lipid chain or it may be terminally located at the terminal end of the lipid chain. The internal ester linkage may replace any carbon in the lipid chain.

In one embodiment, the internal ester linkage may be located on either side of the saturated carbon.

In one embodiment, an immune response may be elicited by delivering a lipid nanoparticle which may include a nanospecies, a polymer and an immunogen. (U.S. Publication No. 20120189700 and International Publication No. WO2012099805; each of which is herein incorporated by reference in their entirety). The polymer may encapsulate the nanospecies or partially encapsulate the nano species. The immunogen may be a recombinant SS18::SSX fusion peptide epitopes and at least one cancer-testis antigen (CTA) epitope, or nucleic acids (e.g., mRNA, cDNA) encoding the same, described herein. In one embodiment, the lipid nanoparticle may be formulated for use in a vaccine such as, but not limited to, against a cancer (e.g., synovial sarcoma).

Lipid nanoparticles may be engineered to alter the surface properties of particles so the lipid nanoparticles may penetrate the mucosal barrier. Mucus is located on mucosal tissue such as, but not limited to, oral (e.g., the buccal and esophageal membranes and tonsil tissue), ophthalmic, gastrointestinal (e.g., stomach, small intestine, large intestine, colon, rectum), nasal, respiratory (e.g., nasal, pharyngeal, tracheal and bronchial membranes), genital (e.g., vaginal, cervical and urethral membranes). Nanoparticles larger than 10-200 nm which are preferred for higher drug encapsulation efficiency and the ability to provide the sustained delivery of a wide array of drugs have been thought to be too large to rapidly diffuse through mucosal barriers. Mucus is continuously secreted, shed, discarded or digested and recycled so most of the trapped particles may be removed from the mucosal tissue within seconds or within a few hours. Large polymeric nanoparticles (200 nm-500 nm in diameter) which have been coated densely with a low molecular weight polyethylene glycol (PEG) diffused through mucus only 4 to 6-fold lower than the same particles diffusing in water (Lai et al. PNAS 2007 104(5):1482-487; Lai et al. Adv Drug Deliv Rev. 2009 61(2): 158-171; each of which is herein incorporated by reference in their entirety). The transport of nanoparticles may be determined using rates of permeation and/or fluorescent microscopy techniques including, but not limited to, fluorescence recovery after photobleaching (FRAP) and high resolution multiple particle tracking (MPT). As a non-limiting example, compositions which can penetrate a mucosal barrier may be made as described in U.S. Pat. No. 8,241,670 or International Patent Publication No. WO2013110028, the contents of each of which are herein incorporated by reference in its entirety.

The lipid nanoparticle engineered to penetrate mucus may comprise a polymeric material (i.e. a polymeric core) and/or a polymer-vitamin conjugate and/or a tri-block co-polymer. The polymeric material may include, but is not limited to, polyamines, polyethers, polyamides, polyesters, polycarbamates, polyureas, polycarbonates, poly(styrenes), polyimides, polysulfones, polyurethanes, polyacetylenes, polyethylenes, polyethyeneimines, polyisocyanates, polyacrylates, polymethacrylates, polyacrylonitriles, and polyarylates. The polymeric material may be biodegradable and/or biocompatible. Non-limiting examples of biocompatible polymers are described in International Patent Publication No. WO2013116804, the contents of which are herein incorporated by reference in its entirety. The polymeric material may additionally be irradiated. As a non-limiting example, the polymeric material may be gamma irradiated (See e.g., International App. No. WO201282165, herein incorporated by reference in its entirety). Non-limiting examples of specific polymers include poly(caprolactone) (PCL), ethylene vinyl acetate polymer (EVA), poly(lactic acid) (PLA), poly(L-lactic acid) (PLLA), poly(glycolic acid) (PGA), poly(lactic acid-co-glycolic acid) (PLGA), poly(L-lactic acid-co-glycolic acid) (PLLGA), poly(D,L-lactide) (PDLA), poly(L-lactide) (PLLA), poly(D,L-lactide-co-caprolactone), poly(D,L-lactide-co-caprolactone-co-glycolide), poly(D,L-lactide-co-PEO-co-D,L-lactidc), poly(D,L-lactide-co-PPO-co-D,L-lactide), polyalkyl cyanoacralate, polyurethane, poly-L-lysine (PLL), hydroxypropyl methacrylate (HPMA), polyethyleneglycol, poly-L-glutamic acid, poly(hydroxy acids), polyanhydrides, polyorthoesters, poly(ester amides), polyamides, poly(ester ethers), polycarbonates, polyalkylenes such as polyethylene and polypropylene, polyalkylene glycols such as poly(ethylene glycol) (PEG), polyalkylene oxides (PEO), polyalkylene terephthalates such as poly(ethylene terephthalate), polyvinyl alcohols (PVA), polyvinyl ethers, polyvinyl esters such as poly(vinyl acetate), polyvinyl halides such as poly(vinyl chloride) (PVC), polyvinylpyrrolidone, polysiloxanes, polystyrene (PS), polyurethanes, derivatized celluloses such as alkyl celluloses, hydroxyalkyl celluloses, cellulose ethers, cellulose esters, nitro celluloses, hydroxypropylcellulose, carboxymethylcellulose, polymers of acrylic acids, such as poly(methyl(meth)acrylate) (PMMA), poly(ethyl(meth)acrylate), poly(butyl(meth)acrylate), poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutyl acrylate), poly(octadecyl acrylate) and copolymers and mixtures thereof, polydioxanone and its copolymers, polyhydroxyalkanoates, polypropylene fumarate, polyoxymethylene, poloxamers, poly(ortho)esters, poly(butyric acid), poly(valeric acid), poly(lactide-co-caprolactone), PEG-PLGA-PEG and trimethylene carbonate, polyvinylpyrrolidone. The lipid nanoparticle may be coated or associated with a co-polymer such as, but not limited to, a block co-polymer (such as a branched polyether-polyamide block copolymer described in International Publication No. WO2013012476, herein incorporated by reference in its entirety), and (poly(ethylene glycol))-(poly(propylene oxide))-(poly(ethylene glycol)) triblock copolymer (see e.g., US Publication 20120121718 and US Publication 20100003337 and U.S. Pat. No. 8,263,665; each of which is herein incorporated by reference in their entirety). The co-polymer may be a polymer that is generally regarded as safe (GRAS) and the formation of the lipid nanoparticle may be in such a way that no new chemical entities are created. For example, the lipid nanoparticle may comprise poloxamers coating PLGA nanoparticles without forming new chemical entities which are still able to rapidly penetrate human mucus (Yang et al. Angew. Chem. Int. Ed. 2011 50:2597-2600; the contents of which are herein incorporated by reference in its entirety). A non-limiting scalable method to produce nanoparticles which can penetrate human mucus is described by Xu et al. (Sec e.g., J Control Release 2013, 170(2):279-86; the contents of which are herein incorporated by reference in its entirety).

The vitamin of the polymer-vitamin conjugate may be vitamin E. The vitamin portion of the conjugate may be substituted with other suitable components such as, but not limited to, vitamin A, vitamin E, other vitamins, cholesterol, a hydrophobic moiety, or a hydrophobic component of other surfactants (e.g., sterol chains, fatty acids, hydrocarbon chains and alkylene oxide chains).

The lipid nanoparticle engineered to penetrate mucus may include surface altering agents such as, but not limited to, polynucleotides, anionic proteins (e.g., bovine serum albumin), surfactants (e.g., cationic surfactants such as for example dimethyldioctadecyl-ammonium bromide), sugars or sugar derivatives (e.g., cyclodextrin), nucleic acids, polymers (e.g., heparin, polyethylene glycol and poloxamer), mucolytic agents (e.g., N-acetylcysteine, mugwort, bromelain, papain, clerodendrum, acetylcysteine, bromhexine, carbocisteine, eprazinone, mesna, ambroxol, sobrerol, domiodol, letosteine, stepronin, tiopronin, gelsolin, thymosin β4 dornase alfa, neltenexine, erdosteine) and various DNases including rhDNase. The surface altering agent may be embedded or enmeshed in the particle's surface or disposed (e.g., by coating, adsorption, covalent linkage, or other process) on the surface of the lipid nanoparticle. (see e.g., US Publication 20100215580 and US Publication 20080166414 and US20130164343; the contents of each of which is herein incorporated by reference in their entirety).

In one embodiment, the mucus penetrating lipid nanoparticles may comprise at least one recombinant SS18::SSX fusion peptide epitopes and at least one cancer-testis antigen (CTA) epitope, or nucleic acids (e.g., mRNA, cDNA) encoding the same, described herein. The recombinant SS18::SSX fusion peptide epitopes and at least one cancer-testis antigen (CTA) epitope, or nucleic acids (e.g., mRNA, cDNA) encoding the same, may be encapsulated in the lipid nanoparticle and/or disposed on the surface of the particle. The recombinant SS18::SSX fusion peptide epitopes and at least one cancer-testis antigen (CTA) epitope, or nucleic acids (e.g., mRNA, cDNA) encoding the same, may be covalently coupled to the lipid nanoparticle. Formulations of mucus penetrating lipid nanoparticles may comprise a plurality of nanoparticles. Further, the formulations may contain particles which may interact with the mucus and alter the structural and/or adhesive properties of the surrounding mucus to decrease mucoadhesion which may increase the delivery of the mucus penetrating lipid nanoparticles to the mucosal tissue.

In another embodiment, the mucus penetrating lipid nanoparticles may be a hypotonic formulation comprising a mucosal penetration enhancing coating. The formulation may be hypotonice for the epithelium to which it is being delivered. Non-limiting examples of hypotonic formulations may be found in International Patent Publication No. WO2013110028, the contents of which are herein incorporated by reference in its entirety.

In one embodiment, in order to enhance the delivery through the mucosal barrier the recombinant SS18::SSX fusion peptide epitopes and at least one cancer-testis antigen (CTA) epitope, or nucleic acids (e.g., mRNA, cDNA) encoding the same, formulation may comprise or be a hypotonic solution. Hypotonic solutions were found to increase the rate at which mucoinert particles such as, but not limited to, mucus-penetrating particles, were able to reach the vaginal epithelial surface (See e.g., Ensign et al. Biomaterials 2013 34(28):6922-9; the contents of which is herein incorporated by reference in its entirety).

In one embodiment, the recombinant SS18::SSX fusion peptide epitopes and at least one cancer-testis antigen (CTA) epitope, or nucleic acids (e.g., mRNA, cDNA) encoding the same, is formulated as a lipoplex, such as, without limitation, the ATUPLEX™ system, the DACC system, the DBTC system and other siRNA-lipoplex technology from Silence Therapeutics (London, United Kingdom), STEMFECT™ from STEMGENT® (Cambridge, Mass.), and polyethylenimine (PEI) or protamine-based targeted and non-targeted delivery of nucleic acids acids (Aleku et al. Cancer Res. 2008 68:9788-9798; Strumberg et al. Int J Clin Pharmacol Ther 2012 50:76-78; Santel et al., Gene Ther 2006 13:1222-1234; Santel et al., Gene Ther 2006 13:1360-1370; Gutbier et al., Pulm Pharmacol. Ther. 2010 23:334-344; Kaufmann et al. Microvasc Res 2010 80:286-293 Weide et al. J Immunother. 2009 32:498-507; Weide et al. J Immunother. 2008 31:180-188; Pascolo Expert Opin. Biol. Ther. 4:1285-1294; Fotin-Mleczek et al., 2011 J. Immunother. 34:1-15; Song et al., Nature Biotechnol. 2005, 23:709-717; Peer et al., Proc Natl Acad Sci USA. 2007 6; 104:4095-4100; deFougerolles Hum Gene Ther. 2008 19:125-132; all of which are incorporated herein by reference in its entirety).

In one embodiment such formulations may also be constructed or compositions altered such that they passively or actively are directed to different cell types in vivo, including but not limited to hepatocytes, immune cells, tumor cells, endothelial cells, antigen presenting cells, and leukocytes (Akinc et al. Mol Ther. 2010 18:1357-1364; Song et al., Nat Biotechnol. 2005 23:709-717; Judge et al., J Clin Invest. 2009 119:661-673; Kaufmann et al., Microvasc Res 2010 80:286-293; Santel et al., Gene Ther 2006 13:1222-1234; Santel et al., Gene Ther 2006 13:1360-1370; Gutbier et al., Pulm Pharmacol. Ther. 2010 23:334-344; Basha et al., Mol. Ther. 2011 19:2186-2200; Fenske and Cullis, Expert Opin Drug Deliv. 2008 5:25-44; Peer et al., Science. 2008 319:627-630; Peer and Lieberman, Gene Ther. 2011 18:1127-1133; all of which are incorporated herein by reference in its entirety). One example of passive targeting of formulations to liver cells includes the DLin-DMA, DLin-KC2-DMA and DLin-MC3-DMA-based lipid nanoparticle formulations which have been shown to bind to apolipoprotein E and promote binding and uptake of these formulations into hepatocytes in vivo (Akinc et al. Mol Ther. 2010 18:1357-1364; herein incorporated by reference in its entirety). Formulations can also be selectively targeted through expression of different ligands on their surface as exemplified by, but not limited by, folate, transferrin, N-acetylgalactosamine (GalNAc), and antibody targeted approaches (Kolhatkar et al., Curr Drug Discov Technol. 2011 8:197-206; Musacchio and Torchilin, Front Biosci. 2011 16:1388-1412; Yu et al., Mol Membr Biol. 2010 27:286-298; Patil et al., Crit Rev Ther Drug Carrier Syst. 2008 25:1-61; Benoit et al., Biomacromolecules. 2011 12:2708-2714; Zhao et al., Expert Opin Drug Deliv. 2008 5:309-319; Akinc et al., Mol Ther. 2010 18:1357-1364; Srinivasan et al., Methods Mol Biol. 2012 820:105-116; Ben-Arie et al., Methods Mol Biol. 2012 757:497-507; Peer 2010 J Control Release. 20:63-68; Peer et al., Proc Natl Acad Sci USA. 2007 104:4095-4100; Kim et al., Methods Mol Biol. 2011 721:339-353; Subramanya et al., Mol Ther. 2010 18:2028-2037; Song et al., Nat Biotechnol. 2005 23:709-717; Peer et al., Science. 2008 319:627-630; Peer and Lieberman, Gene Ther. 2011 18:1127-1133; all of which are incorporated herein by reference in its entirety).

In one embodiment, the recombinant SS18::SSX fusion peptide epitopes and at least one cancer-testis antigen (CTA) epitope, or nucleic acids (e.g., mRNA, cDNA) encoding the same, is formulated as a solid lipid nanoparticle. A solid lipid nanoparticle (SLN) may be spherical with an average diameter between 10 to 1000 nm. SLN possess a solid lipid core matrix that can solubilize lipophilic molecules and may be stabilized with surfactants and/or emulsifiers. In a further embodiment, the lipid nanoparticle may be a self-assembly lipid-polymer nanoparticle (see Zhang et al., ACS Nano, 2008, 2 (8), pp 1696-1702; the contents of which are herein incorporated by reference in its entirety). As a non-limiting example, the SLN may be the SLN described in International Patent Publication No. WO2013105101, the contents of which are herein incorporated by reference in its entirety. As another non-limiting example, the SLN may be made by the methods or processes described in International Patent Publication No. WO2013105101, the contents of which are herein incorporated by reference in its entirety.

Liposomes, lipoplexes, or lipid nanoparticles may be used to improve the efficacy of nucleic acids encoding recombinant SS18::SSX fusion peptide epitopes and at least one cancer-testis antigen (CTA) epitope directed protein production as these formulations may be able to increase cell transfection by the nucleic acids encoding recombinant SS18::SSX fusion peptide epitopes and at least one cancer-testis antigen (CTA) epitope; and/or increase the translation of encoded protein. One such example involves the use of lipid encapsulation to enable the effective systemic delivery of polyplex plasmid DNA (Heyes et al., Mol Ther. 2007 15:713-720; herein incorporated by reference in its entirety). The liposomes, lipoplexes, or lipid nanoparticles may also be used to increase the stability of the polynucleotide.

In one embodiment, the recombinant SS18::SSX fusion peptide epitopes and at least one cancer-testis antigen (CTA) epitope, or nucleic acids (e.g., mRNA, cDNA) encoding the same, of the present disclosure can be formulated for controlled release and/or targeted delivery. As used herein, “controlled release” refers to a pharmaceutical composition or compound release profile that conforms to a particular pattern of release to effect a therapeutic outcome. In one embodiment, the recombinant SS18::SSX fusion peptide epitopes and at least one cancer-testis antigen (CTA) epitope, or nucleic acids (e.g., mRNA, cDNA) encoding the same, may be encapsulated into a delivery agent described herein and/or known in the art for controlled release and/or targeted delivery. As used herein, the term “encapsulate” means to enclose, surround or encase. As it relates to the formulation of the compounds of the present disclosure, encapsulation may be substantial, complete or partial. The term “substantially encapsulated” means that at least greater than 50, 60, 70, 80, 85, 90, 95, 96, 97, 98, 99, 99.9, 99.9 or greater than 99.999% of the pharmaceutical composition or compound of the present disclosure may be enclosed, surrounded or encased within the delivery agent. “Partially encapsulation” means that less than 10, 10, 20, 30, 40 50 or less of the pharmaceutical composition or compound of the present disclosure may be enclosed, surrounded or encased within the delivery agent. Advantageously, encapsulation may be determined by measuring the escape or the activity of the pharmaceutical composition or compound of the present disclosure using fluorescence and/or electron micrograph. For example, at least 1, 5, 10, 20, 30, 40, 50, 60, 70, 80, 85, 90, 95, 96, 97, 98, 99, 99.9, 99.99 or greater than 99.99% of the pharmaceutical composition or compound of the present disclosure are encapsulated in the delivery agent.

In one embodiment, the controlled release formulation may include, but is not limited to, tri-block co-polymers. As a non-limiting example, the formulation may include two different types of tri-block co-polymers (International Pub. No. WO2012131104 and WO2012131106; the contents of each of which is herein incorporated by reference in its entirety).

In another embodiment, the recombinant SS18::SSX fusion peptide epitopes and at least one cancer-testis antigen (CTA) epitope, or nucleic acids (e.g., mRNA, cDNA) encoding the same, may be encapsulated into a lipid nanoparticle or a rapidly eliminated lipid nanoparticle and the lipid nanoparticles or a rapidly eliminated lipid nanoparticle may then be encapsulated into a polymer, hydrogel and/or surgical sealant described herein and/or known in the art. As a non-limiting example, the polymer, hydrogel or surgical sealant may be PLGA, ethylene vinyl acetate (EVAc), poloxamer, GELSITE® (Nanotherapeutics, Inc. Alachua, Fla.), HYLENEX® (Halozyme Therapeutics, San Diego Calif), surgical sealants such as fibrinogen polymers (Ethicon Inc. Cornelia, Ga.), TISSELL® (Baxter International, Inc Deerfield, Ill.), PEG-based sealants, and COSEAL® (Baxter International, Inc Deerfield, Ill.).

In another embodiment, the lipid nanoparticle may be encapsulated into any polymer known in the art which may form a gel when injected into a subject. As another non-limiting example, the lipid nanoparticle may be encapsulated into a polymer matrix which may be biodegradable.

In one embodiment, the recombinant SS18::SSX fusion peptide epitopes and at least one cancer-testis antigen (CTA) epitope, or nucleic acids (e.g., mRNA, cDNA) encoding the same, formulation for controlled release and/or targeted delivery may also include at least one controlled release coating. Controlled release coatings include, but are not limited to, OPADRY®, polyvinylpyrrolidone/vinyl acetate copolymer, polyvinylpyrrolidone, hydroxypropyl methylcellulose, hydroxypropyl cellulose, hydroxyethyl cellulose, EUDRAGIT RL®, EUDRAGIT RS® and cellulose derivatives such as ethylcellulose aqueous dispersions (AQUACOAT® and SURELEASE®).

In one embodiment, the recombinant SS18::SSX fusion peptide epitopes and at least one cancer-testis antigen (CTA) epitope, or nucleic acids (e.g., mRNA, cDNA) encoding the same, controlled release and/or targeted delivery formulation may comprise at least one degradable polyester which may contain polycationic side chains. Degradable polyesters include, but are not limited to, poly(serine ester), poly(L-lactide-co-L-lysine), poly(4-hydroxy-L-proline ester), and combinations thereof. In another embodiment, the degradable polyesters may include a PEG conjugation to form a PEGylated polymer.

In one embodiment, the recombinant SS18::SSX fusion peptide epitopes and at least one cancer-testis antigen (CTA) epitope, or nucleic acids (e.g., mRNA, cDNA) encoding the same, controlled release and/or targeted delivery formulation comprising at least one polynucleotide may comprise at least one PEG and/or PEG related polymer derivatives as described in U.S. Pat. No. 8,404,222, herein incorporated by reference in its entirety.

In another embodiment, the recombinant SS18::SSX fusion peptide epitopes and at least one cancer-testis antigen (CTA) epitope, or nucleic acids (e.g., mRNA, cDNA) encoding the same, controlled release delivery formulation may be the controlled release polymer system described in US20130130348, herein incorporated by reference in its entirety.

In one embodiment, the recombinant SS18::SSX fusion peptide epitopes and at least one cancer-testis antigen (CTA) epitope, or nucleic acids (e.g., mRNA, cDNA) encoding the same, of the present disclosure may be encapsulated in a therapeutic nanoparticle, referred to herein as “therapeutic nanoparticle recombinant SS18::SSX fusion peptide epitopes and at least one cancer-testis antigen (CTA) epitope, or nucleic acids (e.g., mRNA, cDNA) encoding the same.” Therapeutic nanoparticles may be formulated by methods described herein and known in the art such as, but not limited to, International Pub Nos. WO2010005740, WO2010030763, WO2010005721, WO2010005723, WO2012054923, US Pub. Nos. US20110262491, US20100104645, US20100087337, US20100068285, US20110274759, US20100068286, US20120288541, US20130123351 and US20130230567 and U.S. Pat. Nos. 8,206,747, 8,293,276, 8,318,208 and 8,318,211; the contents of each of which are herein incorporated by reference in their entirety. In another embodiment, therapeutic polymer nanoparticles may be identified by the methods described in US Pub No. US20120140790, the contents of which is herein incorporated by reference in its entirety.

In one embodiment, the therapeutic nanoparticle recombinant SS18::SSX fusion peptide epitopes and at least one cancer-testis antigen (CTA) epitope, or nucleic acids (e.g., mRNA, cDNA) encoding the same, may be formulated for sustained release. As used herein, “sustained release” refers to a pharmaceutical composition or compound that conforms to a release rate over a specific period of time. The period of time may include, but is not limited to, hours, days, weeks, months and years. As a non-limiting example, the sustained release nanoparticle may comprise a polymer and a therapeutic agent such as, but not limited to, the polynucleotides of the present disclosure (see International Pub No. 2010075072 and US Pub No. US20100216804, US20110217377 and US20120201859, each of which is herein incorporated by reference in their entirety). In another non-limiting example, the sustained release formulation may comprise agents which permit persistent bioavailability such as, but not limited to, crystals, macromolecular gels and/or particulate suspensions (see US Patent Publication No US20130150295, the contents of which is herein incorporated by reference in its entirety).

In one embodiment, the therapeutic nanoparticle recombinant SS18::SSX fusion peptide epitopes and at least one cancer-testis antigen (CTA) epitope, or nucleic acids (e.g., mRNA, cDNA) encoding the same, may be formulated to be target specific. As a non-limiting example, the therapeutic nanoparticles may include a corticosteroid (see International Pub. No. WO2011084518; herein incorporated by reference in its entirety). As a non-limiting example, the therapeutic nanoparticles may be formulated in nanoparticles described in International Pub No. WO2008121949, WO2010005726, WO2010005725, WO2011084521 and US Pub No. US20100069426, US20120004293 and US20100104655, each of which is herein incorporated by reference in their entirety.

In one embodiment, the nanoparticles of the present disclosure may comprise a polymeric matrix. As a non-limiting example, the nanoparticle may comprise two or more polymers such as, but not limited to, polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, polyamines, polylysinc, poly(ethylene imine), poly(serine ester), poly(L-lactide-co-L-lysine), poly(4-hydroxy-L-proline ester) or combinations thereof.

In one embodiment, the therapeutic nanoparticle comprises a diblock copolymer. In one embodiment, the diblock copolymer may include PEG in combination with a polymer such as, but not limited to, polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, polyamines, polylysinc, poly(ethylene imine), poly(serine ester), poly(L-lactide-co-L-lysine), poly(4-hydroxy-L-proline ester) or combinations thereof. In another embodiment, the diblock copolymer may comprise the diblock copolymers described in European Patent Publication No. the contents of which are herein incorporated by reference in its entirety. In yet another embodiment, the diblock copolymer may be a high-X diblock copolymer such as those described in International Patent Publication No. WO2013120052, the contents of which are herein incorporated by reference in its entirety.

As a non-limiting example the therapeutic nanoparticle comprises a PLGA-PEG block copolymer (see US Pub. No. US20120004293 and U.S. Pat. No. 8,236,330, each of which is herein incorporated by reference in their entirety). In another non-limiting example, the therapeutic nanoparticle is a stealth nanoparticle comprising a diblock copolymer of PEG and PLA or PEG and PLGA (see U.S. Pat. No. 8,246,968 and International Publication No. WO2012166923, the contents of each of which are herein incorporated by reference in its entirety). In yet another non-limiting example, the therapeutic nanoparticle is a stealth nanoparticle or a target-specific stealth nanoparticle as described in US Patent Publication No. US20130172406, the contents of which are herein incorporated by reference in its entirety.

In one embodiment, the therapeutic nanoparticle may comprise a multiblock copolymer (See e.g., U.S. Pat. Nos. 8,263,665 and 8,287,910 and US Patent Pub. No. US20130195987; the contents of each of which are herein incorporated by reference in its entirety).

In yet another non-limiting example, the lipid nanoparticle comprises the block copolymer PEG-PLGA-PEG (see e.g., the thermosensitive hydrogel (PEG-PLGA-PEG) was used as a TGF-betal gene delivery vehicle in Lee et al. Thermosensitive Hydrogel as a Tgf-β1 Gene Delivery Vehicle Enhances Diabetic Wound Healing. Pharmaceutical Research, 2003 20(12): 1995-2000; as a controlled gene delivery system in Li et al. Controlled Gene Delivery System Based on Thermosensitive Biodegradable Hydrogel. Pharmaceutical Research 2003 20(6):884-888; and Chang et al., Non-ionic amphiphilic biodegradable PEG-PLGA-PEG copolymer enhances gene delivery efficiency in rat skeletal muscle. J Controlled Release. 2007 118:245-253; each of which is herein incorporated by reference in its entirety). The recombinant SS18::SSX fusion peptide epitopes and at least one cancer-testis antigen (CTA) epitope, or nucleic acids (e.g., mRNA, cDNA) encoding the same, of the present disclosure may be formulated in lipid nanoparticles comprising the PEG-PLGA-PEG block copolymer.

In one embodiment, the therapeutic nanoparticle may comprise a multiblock copolymer (See e.g., U.S. Pat. Nos. 8,263,665 and 8,287,910 and US Patent Pub. No. US20130195987; the contents of each of which are herein incorporated by reference in its entirety).

In one embodiment, the block copolymers described herein may be included in a polyion complex comprising a non-polymeric micelle and the block copolymer. (See e.g., U.S. Pub. No. 20120076836; herein incorporated by reference in its entirety).

In one embodiment, the therapeutic nanoparticle may comprise at least one acrylic polymer. Acrylic polymers include but are not limited to, acrylic acid, methacrylic acid, acrylic acid and methacrylic acid copolymers, methyl methacrylate copolymers, ethoxyethyl methacrylates, cyanoethyl methacrylate, amino alkyl methacrylate copolymer, poly(acrylic acid), poly(methacrylic acid), polycyanoacrylates and combinations thereof.

In one embodiment, the therapeutic nanoparticles may comprise at least one poly(vinyl ester) polymer. The poly(vinyl ester) polymer may be a copolymer such as a random copolymer. As a non-limiting example, the random copolymer may have a structure such as those described in International Application No. WO2013032829 or US Patent Publication No US20130121954, the contents of which are herein incorporated by reference in its entirety. In one aspect, the poly(vinyl ester) polymers may be conjugated to the recombinant SS18::SSX fusion peptide epitopes and at least one cancer-testis antigen (CTA) epitope, or nucleic acids (e.g., mRNA, cDNA) encoding the same, described herein. In another aspect, the poly(vinyl ester) polymer which may be used in the present disclosure may be those described in, herein incorporated by reference in its entirety.

In one embodiment, the therapeutic nanoparticle may comprise at least one diblock copolymer. The diblock copolymer may be, but it not limited to, a poly(lactic) acid-poly(ethylene)glycol copolymer (see e.g., International Patent Publication No. WO2013044219; herein incorporated by reference in its entirety). As a non-limiting example, the therapeutic nanoparticle may be used to treat cancer (see International publication No. WO2013044219; herein incorporated by reference in its entirety).

In one embodiment, the therapeutic nanoparticles may comprise at least one cationic polymer described herein and/or known in the art.

In one embodiment, the therapeutic nanoparticles may comprise at least one amine-containing polymer such as, but not limited to polylysine, polyethylene imine, poly(amidoamine) dendrimers, poly(beta-amino esters) (Sec e.g., U.S. Pat. No. 8,287,849; herein incorporated by reference in its entirety) and combinations thereof.

In another embodiment, the nanoparticles described herein may comprise an amine cationic lipid such as those described in International Patent Application No. WO2013059496, the contents of which are herein incorporated by reference in its entirety. In one aspect the cationic lipids may have an amino-amine or an amino-amide moiety.

In one embodiment, the therapeutic nanoparticles may comprise at least one degradable polyester which may contain polycationic side chains. Degradable polyesters include, but are not limited to, poly(serine ester), poly(L-lactide-co-L-lysine), poly(4-hydroxy-L-proline ester), and combinations thereof. In another embodiment, the degradable polyesters may include a PEG conjugation to form a PEGylated polymer.

In another embodiment, the therapeutic nanoparticle may include a conjugation of at least one targeting ligand. The targeting ligand may be any ligand known in the art such as, but not limited to, a monoclonal antibody. (Kirpotin et al, Cancer Res. 2006 66:6732-6740; herein incorporated by reference in its entirety).

In one embodiment, the therapeutic nanoparticle may be formulated in an aqueous solution which may be used to target cancer (see International Pub No. WO2011084513 and US Pub No. US20110294717, each of which is herein incorporated by reference in their entirety).

In one embodiment, the therapeutic nanoparticle recombinant SS18::SSX fusion peptide epitopes and at least one cancer-testis antigen (CTA) epitope, or nucleic acids (e.g., mRNA, cDNA) encoding the same, e.g., therapeutic nanoparticles comprising at least one recombinant SS18::SSX fusion peptide epitopes and at least one cancer-testis antigen (CTA) epitope, or nucleic acids (e.g., mRNA, cDNA) encoding the same, may be formulated using the methods described by Podobinski et al in U.S. Pat. No. 8,404,799, the contents of which are herein incorporated by reference in its entirety.

In one embodiment, the recombinant SS18::SSX fusion peptide epitopes and at least one cancer-testis antigen (CTA) epitope, or nucleic acids (e.g., mRNA, cDNA) encoding the same, may be encapsulated in, linked to and/or associated with synthetic nanocarriers. Synthetic nanocarriers include, but are not limited to, those described in International Pub. Nos. WO2010005740, WO2010030763, WO201213501, WO2012149252, WO2012149255, WO2012149259, WO2012149265, WO2012149268, WO2012149282, WO2012149301, WO2012149393, WO2012149405, WO2012149411, WO2012149454 and WO2013019669, and US Pub. Nos. US20110262491, US20100104645, US20100087337 and US20120244222, each of which is herein incorporated by reference in their entirety. The synthetic nanocarriers may be formulated using methods known in the art and/or described herein. As a non-limiting example, the synthetic nanocarriers may be formulated by the methods described in International Pub Nos. WO2010005740, WO2010030763 and WO201213501 and US Pub. Nos. US20110262491, US20100104645, US20100087337 and US2012024422, each of which is herein incorporated by reference in their entirety. In another embodiment, the synthetic nanocarrier formulations may be lyophilized by methods described in International Pub. No. WO2011072218 and U.S. Pat. No. 8,211,473; the content of each of which is herein incorporated by reference in their entirety. In yet another embodiment, formulations of the present disclosure, including, but not limited to, synthetic nanocarriers, may be lyophilized or reconstituted by the methods described in US Patent Publication No. US20130230568, the contents of which are herein incorporated by reference in its entirety.

In one embodiment, the synthetic nanocarriers may contain reactive groups to release the recombinant SS18::SSX fusion peptide epitopes and at least one cancer-testis antigen (CTA) epitope, or nucleic acids (e.g., mRNA, cDNA) encoding the same, described herein (see International Pub. No. WO20120952552 and US Pub No. US20120171229, each of which is herein incorporated by reference in their entirety).

In one embodiment, the synthetic nanocarriers may contain an immunostimulatory agent to enhance the immune response from delivery of the synthetic nanocarrier. As a non-limiting example, the synthetic nanocarrier may comprise a Th1 immunostimulatory agent which may enhance a Th1-based response of the immune system (see International Pub No. WO2010123569 and US Pub. No. US20110223201, each of which is herein incorporated by reference in its entirety).

In one embodiment, the synthetic nanocarriers may be formulated for targeted release. In one embodiment, the synthetic nanocarrier is formulated to release the recombinant SS18::SSX fusion peptide epitopes and at least one cancer-testis antigen (CTA) epitope, or nucleic acids (e.g., mRNA, cDNA) encoding the same, at a specified pH and/or after a desired time interval. As a non-limiting example, the synthetic nanoparticle may be formulated to release the recombinant SS18::SSX fusion peptide epitopes and at least one cancer-testis antigen (CTA) epitope, or nucleic acids (e.g., mRNA, cDNA) encoding the same, after 24 hours and/or at a pH of 4.5 (see International Pub. Nos. WO2010138193 and WO2010138194 and US Pub Nos. US20110020388 and US20110027217, each of which is herein incorporated by reference in their entireties).

In one embodiment, the synthetic nanocarriers may be formulated for controlled and/or sustained release of the recombinant SS18::SSX fusion peptide epitopes and at least one cancer-testis antigen (CTA) epitope, or nucleic acids (e.g., mRNA, cDNA) encoding the same, described herein. As a non-limiting example, the synthetic nanocarriers for sustained release may be formulated by methods known in the art, described herein and/or as described in International Pub No. WO2010138192 and US Pub No. 20100303850, each of which is herein incorporated by reference in their entirety.

In one embodiment, the recombinant SS18::SSX fusion peptide epitopes and at least one cancer-testis antigen (CTA) epitope, or nucleic acids (e.g., mRNA, cDNA) encoding the same, may be formulated for controlled and/or sustained release wherein the formulation comprises at least one polymer that is a crystalline side chain (CYSC) polymer. CYSC polymers are described in U.S. Pat. No. 8,399,007, herein incorporated by reference in its entirety.

In one embodiment, the synthetic nanocarrier may be formulated for use as a vaccine. In one embodiment, the synthetic nanocarrier may encapsulate at least one recombinant SS18::SSX fusion peptide epitopes and at least one cancer-testis antigen (CTA) epitope, or nucleic acids (e.g., mRNA, cDNA) encoding the same. As a non-limiting example, the synthetic nanocarrier may include at least one recombinant SS18::SSX fusion peptide epitopes and at least one cancer-testis antigen (CTA) epitope, or nucleic acids (e.g., mRNA, cDNA) encoding the same, and an excipient for a vaccine dosage form (see International Pub No. WO2011150264 and US Pub No. US20110293723, each of which is herein incorporated by reference in their entirety). As another non-limiting example, a vaccine dosage form may include at least two synthetic nanocarriers with the same or different recombinant SS18::SSX fusion peptide epitopes and at least one cancer-testis antigen (CTA) epitope, or nucleic acids (e.g., mRNA, cDNA) encoding the same, and an excipient (see International Pub No. WO2011150249 and US Pub No. US20110293701, each of which is herein incorporated by reference in their entirety). The vaccine dosage form may be selected by methods described herein, known in the art and/or described in International Pub No. WO2011150258 and US Pub No. US20120027806, each of which is herein incorporated by reference in their entirety).

In one embodiment, the synthetic nanocarrier may comprise at least one polynucleotide which encodes at least one adjuvant. As non-limiting example, the adjuvant may comprise dimethyldioctadecylammonium-bromide, dimethyldioctadecylammonium-chloride, dimethyldioctadecylammonium-phosphate or dimethyldioctadecylammonium-acetate (DDA) and an apolar fraction or part of said apolar fraction of a total lipid extract of a Mycobacterium (See e.g, U.S. Pat. No. 8,241,610; herein incorporated by reference in its entirety). In another embodiment, the synthetic nanocarrier may comprise at least one recombinant SS18::SSX fusion peptide epitopes and at least one cancer-testis antigen (CTA) epitope, or nucleic acids (e.g., mRNA, cDNA) encoding the same, and an adjuvant. As a non-limiting example, the synthetic nanocarrier comprising an adjuvant may be formulated by the methods described in International Pub No. WO2011150240 and US Pub No. US20110293700, each of which is herein incorporated by reference in its entirety.

In one embodiment, the synthetic nanocarrier may encapsulate at least one recombinant SS18::SSX fusion peptide epitopes and at least one cancer-testis antigen (CTA) epitope, or nucleic acids (e.g., mRNA, cDNA) encoding the same. As a non-limiting example, the synthetic nanocarrier may include, but is not limited to, the nanocarriers described in International Pub No. WO2012024621, WO201202629, WO2012024632 and US Pub No. US20120064110, US20120058153 and US20120058154, each of which is herein incorporated by reference in their entirety.

In one embodiment, the synthetic nanocarrier may be coupled to a polynucleotide which may be able to trigger a humoral and/or cytotoxic T lymphocyte (CTL) response (See e.g., International Publication No. WO2013019669, herein incorporated by reference in its entirety).

In one embodiment, the recombinant SS18::SSX fusion peptide epitopes and at least one cancer-testis antigen (CTA) epitope, or nucleic acids (e.g., mRNA, cDNA) encoding the same, may be encapsulated in, linked to and/or associated with zwitterionic lipids. Non-limiting examples of zwitterionic lipids and methods of using zwitterionic lipids are described in US Patent Publication No. US20130216607, the contents of which are herein incorporated by reference in its entirety. In one aspect, the zwitterionic lipids may be used in the liposomes and lipid nanoparticles described herein.

In one embodiment, the recombinant SS18::SSX fusion peptide epitopes and at least one cancer-testis antigen (CTA) epitope, or nucleic acids (e.g., mRNA, cDNA) encoding the same, may be formulated in colloid nanocarriers as described in US Patent Publication No. US20130197100, the contents of which are herein incorporated by reference in its entirety.

In one embodiment, the nanoparticle may be optimized for oral administration. The nanoparticle may comprise at least one cationic biopolymer such as, but not limited to, chitosan or a derivative thereof. As a non-limiting example, the nanoparticle may be formulated by the methods described in U.S. Pub. No. 20120282343; herein incorporated by reference in its entirety.

In some embodiments, LNPs comprise the lipid KL52 (an amino-lipid disclosed in U.S. Application Publication No. 2012/0295832 expressly incorporated herein by reference in its entirety). Activity and/or safety (as measured by examining one or more of ALT/AST, white blood cell count and cytokine induction) of LNP administration may be improved by incorporation of such lipids. LNPs comprising KL52 may be administered intravenously and/or in one or more doses. In some embodiments, administration of LNPs comprising KL52 results in equal or improved mRNA and/or protein expression as compared to LNPs comprising MC3.

In some embodiments, recombinant SS18::SSX fusion peptide epitopes and at least one cancer-testis antigen (CTA) epitope, or nucleic acids (e.g., mRNA, cDNA) encoding the same, may be delivered using smaller LNPs. Such particles may comprise a diameter from below 0.1 μm up to 100 nm such as, but not limited to, less than 0.1 μm, less than 1.0 μm, less than 5 μm, less than 10 μm, less than 15 μm, less than 20 μm, less than 25 μm, less than 30 μm, less than 35 μm, less than 40 μm, less than 50 μm, less than 55 μm, less than 60 μm, less than 65 μm, less than 70 μm, less than 75 μm, less than 80 μm, less than 85 μm, less than 90 m, less than 95 μm, less than 100 μm, less than 125 μm, less than 150 μm, less than 175 μm, less than 200 μm, less than 225 μm, less than 250 μm, less than 275 μm, less than 300 μm, less than 325 μm, less than 350 μm, less than 375 μm, less than 400 μm, less than 425 μm, less than 450 μm, less than 475 μm, less than 500 μm, less than 525 μm, less than 550 m, less than 575 μm, less than 600 μm, less than 625 μm, less than 650 μm, less than 675 μm, less than 700 μm, less than 725 μm, less than 750 μm, less than 775 μm, less than 800 μm, less than 825 μm, less than 850 μm, less than 875 μm, less than 900 μm, less than 925 μm, less than 950 μm, or less than 975 μm.

In another embodiment, recombinant SS18::SSX fusion peptide epitopes and at least one cancer-testis antigen (CTA) epitope, or nucleic acids (e.g., mRNA, cDNA) encoding the same, may be delivered using smaller LNPs which may comprise a diameter from about 1 nm to about 100 nm, from about 1 nm to about 10 nm, about 1 nm to about 20 nm, from about 1 nm to about 30 nm, from about 1 nm to about 40 nm, from about 1 nm to about 50 nm, from about 1 nm to about 60 nm, from about 1 nm to about 70 nm, from about 1 nm to about 80 nm, from about 1 nm to about 90 nm, from about 5 nm to about from 100 nm, from about 5 nm to about 10 nm, about 5 nm to about 20 nm, from about 5 nm to about 30 nm, from about 5 nm to about 40 nm, from about 5 nm to about 50 nm, from about 5 nm to about 60 nm, from about 5 nm to about 70 nm, from about 5 nm to about 80 nm, from about 5 nm to about 90 nm, about 10 to about 50 nM, from about 20 to about 50 nm, from about 30 to about 50 nm, from about 40 to about 50 nm, from about 20 to about 60 nm, from about 30 to about 60 nm, from about 40 to about 60 nm, from about 20 to about 70 nm, from about 30 to about 70 nm, from about 40 to about 70 nm, from about 50 to about 70 nm, from about 60 to about 70 nm, from about 20 to about 80 nm, from about 30 to about 80 nm, from about 40 to about 80 nm, from about 50 to about 80 nm, from about 60 to about 80 nm, from about 20 to about 90 nm, from about 30 to about 90 nm, from about 40 to about 90 nm, from about 50 to about 90 nm, from about 60 to about 90 nm and/or from about 70 to about 90 nm.

In some embodiments, such LNPs are synthesized using methods comprising microfluidic mixers. Exemplary microfluidic mixers may include, but are not limited to a slit interdigitial micromixer including, but not limited to those manufactured by Microinnova (Allerheiligen bei Wildon, Austria) and/or a staggered herringbone micromixer (SHM) (Zhigaltsev, I. V. et al., Bottom-up design and synthesis of limit size lipid nanoparticle systems with aqueous and triglyceride cores using millisecond microfluidic mixing have been published (Langmuir. 2012. 28:3633-40; Belliveau, N. M. et al., Microfluidic synthesis of highly potent limit-size lipid nanoparticles for in vivo delivery of siRNA. Molecular Therapy-Nucleic Acids. 2012. 1:e37; Chen, D. et al., Rapid discovery of potent siRNA-containing lipid nanoparticles enabled by controlled microfluidic formulation. J Am Chem Soc. 2012. 134(16):6948-51; each of which is herein incorporated by reference in its entirety). In some embodiments, methods of LNP generation comprising SHM, further comprise the mixing of at least two input streams wherein mixing occurs by microstructure-induced chaotic advection (MICA). According to this method, fluid streams flow through channels present in a herringbone pattern causing rotational flow and folding the fluids around each other. This method may also comprise a surface for fluid mixing wherein the surface changes orientations during fluid cycling. Methods of generating LNPs using SHM include those disclosed in U.S. Application Publication Nos. 2004/0262223 and 2012/0276209, each of which is expressly incorporated herein by reference in their entirety.

In one embodiment, the recombinant SS18::SSX fusion peptide epitopes and at least one cancer-testis antigen (CTA) epitope, or nucleic acids (e.g., mRNA, cDNA) encoding the same, of the present disclosure may be formulated in lipid nanoparticles created using a micromixer such as, but not limited to, a Slit Interdigital Microstructured Mixer (SIMM-V2) or a Standard Slit Interdigital Micro Mixer (SSIMM) or Caterpillar (CPMM) or Impinging-jet (IJMM) from the Institut für Mikrotechnik Mainz GmbH, Mainz Germany).

In one embodiment, the recombinant SS18::SSX fusion peptide epitopes and at least one cancer-testis antigen (CTA) epitope, or nucleic acids (e.g., mRNA, cDNA) encoding the same, of the present disclosure may be formulated in lipid nanoparticles created using microfluidic technology (see Whitesides, George M. The Origins and the Future of Microfluidics. Nature, 2006 442: 368-373; and Abraham et al. Chaotic Mixer for Microchannels. Science, 2002 295: 647-651; each of which is herein incorporated by reference in its entirety). As a non-limiting example, controlled microfluidic formulation includes a passive method for mixing streams of steady pressure-driven flows in micro channels at a low Reynolds number (See e.g., Abraham et al. Chaotic Mixer for Microchannels. Science, 2002 295: 647-651; which is herein incorporated by reference in its entirety).

In one embodiment, the recombinant SS18::SSX fusion peptide epitopes and at least one cancer-testis antigen (CTA) epitope, or nucleic acids (e.g., mRNA, cDNA) encoding the same, of the present disclosure may be formulated in lipid nanoparticles created using a micromixer chip such as, but not limited to, those from Harvard Apparatus (Holliston, Mass.) or Dolomite Microfluidics (Royston, UK). A micromixer chip can be used for rapid mixing of two or more fluid streams with a split and recombine mechanism.

In one embodiment, the recombinant SS18::SSX fusion peptide epitopes and at least one cancer-testis antigen (CTA) epitope, or nucleic acids (e.g., mRNA, cDNA) encoding the same, of the present disclosure may be formulated for delivery using the drug encapsulating microspheres described in International Patent Publication No. WO2013063468 or U.S. Pat. No. 8,440,614, each of which is herein incorporated by reference in its entirety. The microspheres may comprise a compound of the formula (I), (II), (III), (IV), (V) or (VI) as described in International Patent Publication No. WO2013063468, the contents of which are herein incorporated by reference in its entirety. In another aspect, the amino acid, peptide, polypeptide, lipids (APPL) are useful in delivering the recombinant SS18::SSX fusion peptide epitopes and at least one cancer-testis antigen (CTA) epitope, or nucleic acids (e.g., mRNA, cDNA) encoding the same, of the present disclosure to cells (see International Patent Publication No. WO2013063468, the contents of which is herein incorporated by reference in its entirety).

In one embodiment, the recombinant SS18::SSX fusion peptide epitopes and at least one cancer-testis antigen (CTA) epitope, or nucleic acids (e.g., mRNA, cDNA) encoding the same, of the present disclosure may be formulated in lipid nanoparticles having a diameter from about 10 to about 100 nm such as, but not limited to, about 10 to about 20 nm, about 10 to about 30 nm, about 10 to about 40 nm, about 10 to about 50 nm, about 10 to about 60 nm, about 10 to about 70 nm, about 10 to about 80 nm, about 10 to about 90 nm, about 20 to about 30 nm, about 20 to about 40 nm, about 20 to about 50 nm, about 20 to about 60 nm, about 20 to about 70 nm, about 20 to about 80 nm, about 20 to about 90 nm, about 20 to about 100 nm, about 30 to about 40 nm, about 30 to about 50 nm, about 30 to about 60 nm, about 30 to about 70 nm, about 30 to about 80 nm, about 30 to about 90 nm, about 30 to about 100 nm, about 40 to about 50 nm, about 40 to about 60 nm, about 40 to about 70 nm, about 40 to about 80 nm, about 40 to about 90 nm, about 40 to about 100 nm, about 50 to about 60 nm, about 50 to about 70 nm about 50 to about 80 nm, about 50 to about 90 nm, about 50 to about 100 nm, about 60 to about 70 nm, about 60 to about 80 nm, about 60 to about 90 nm, about 60 to about 100 nm, about 70 to about 80 nm, about 70 to about 90 nm, about 70 to about 100 nm, about 80 to about 90 nm, about 80 to about 100 nm and/or about 90 to about 100 nm.

In one embodiment, the lipid nanoparticles may have a diameter from about 10 to 500 nm or about 50 to 200 nm.

In one embodiment, the lipid nanoparticle may have a diameter greater than 100 nm, greater than 150 nm, greater than 200 nm, greater than 250 nm, greater than 300 nm, greater than 350 nm, greater than 400 nm, greater than 450 nm, greater than 500 nm, greater than 550 nm, greater than 600 nm, greater than 650 nm, greater than 700 nm, greater than 750 nm, greater than 800 nm, greater than 850 nm, greater than 900 nm, greater than 950 nm or greater than 1000 nm.

In one aspect, the lipid nanoparticle may be a limit size lipid nanoparticle described in International Patent Publication No. WO2013059922, the contents of which are herein incorporated by reference in its entirety. The limit size lipid nanoparticle may comprise a lipid bilayer surrounding an aqueous core or a hydrophobic core; where the lipid bilayer may comprise a phospholipid such as, but not limited to, diacylphosphatidylcholine, a diacylphosphatidylethanolamine, a ceramide, a sphingomyelin, a dihydrosphingomyelin, a cephalin, a cerebroside, a C8-C20 fatty acid diacylphophatidylcholine, and 1-palmitoyl-2-oleoyl phosphatidylcholine (POPC). In another aspect the limit size lipid nanoparticle may comprise a polyethylene glycol-lipid such as, but not limited to, DLPE-PEG, DMPE-PEG, DPPC-PEG and DSPE-PEG.

In one embodiment, the recombinant SS18::SSX fusion peptide epitopes and at least one cancer-testis antigen (CTA) epitope, or nucleic acids (e.g., mRNA, cDNA) encoding the same, may be delivered, localized and/or concentrated in a specific location using the delivery methods described in International Patent Publication No. WO2013063530, the contents of which are herein incorporated by reference in its entirety. As a non-limiting example, a subject may be administered an empty polymeric particle prior to, simultaneously with or after delivering the recombinant SS18::SSX fusion peptide epitopes and at least one cancer-testis antigen (CTA) epitope, or nucleic acids (e.g., mRNA, cDNA) encoding the same, to the subject. The empty polymeric particle undergoes a change in volume once in contact with the subject and becomes lodged, embedded, immobilized or entrapped at a specific location in the subject.

In one embodiment, the recombinant SS18::SSX fusion peptide epitopes and at least one cancer-testis antigen (CTA) epitope, or nucleic acids (e.g., mRNA, cDNA) encoding the same, may be formulated in an active substance release system (See e.g., US Patent Publication No. US20130102545, the contents of which is herein incorporated by reference in its entirety). The active substance release system may comprise 1) at least one nanoparticle bonded to an oligonucleotide inhibitor strand which is hybridized with a catalytically active nucleic acid and 2) a compound bonded to at least one substrate molecule bonded to a therapeutically active substance (e.g., recombinant SS18::SSX fusion peptide epitopes and at least one cancer-testis antigen (CTA) epitope, or nucleic acids (e.g., mRNA, cDNA) encoding the same, described herein), where the therapeutically active substance is released by the cleavage of the substrate molecule by the catalytically active nucleic acid.

In one embodiment, the recombinant SS18::SSX fusion peptide epitopes and at least one cancer-testis antigen (CTA) epitope, or nucleic acids (e.g., mRNA, cDNA) encoding the same, may be formulated in a nanoparticle comprising an inner core comprising a non-cellular material and an outer surface comprising a cellular membrane. The cellular membrane may be derived from a cell or a membrane derived from a virus. As a non-limiting example, the nanoparticle may be made by the methods described in International Patent Publication No. WO2013052167, herein incorporated by reference in its entirety. As another non-limiting example, the nanoparticle described in International Patent Publication No. WO2013052167, herein incorporated by reference in its entirety, may be used to deliver the recombinant SS18::SSX fusion peptide epitopes and at least one cancer-testis antigen (CTA) epitope, or nucleic acids (e.g., mRNA, cDNA) encoding the same, described herein.

In one embodiment, the recombinant SS18::SSX fusion peptide epitopes and at least one cancer-testis antigen (CTA) epitope, or nucleic acids (e.g., mRNA, cDNA) encoding the same, may be formulated in porous nanoparticle-supported lipid bilayers (protocells). Protocells are described in International Patent Publication No. WO2013056132, the contents of which are herein incorporated by reference in its entirety.

In one embodiment, the recombinant SS18::SSX fusion peptide epitopes and at least one cancer-testis antigen (CTA) epitope, or nucleic acids (e.g., mRNA, cDNA) encoding the same, described herein may be formulated in polymeric nanoparticles as described in or made by the methods described in U.S. Pat. Nos. 8,420,123 and 8,518,963 and European Patent No. EP2073848B1, the contents of each of which are herein incorporated by reference in their entirety. As a non-limiting example, the polymeric nanoparticle may have a high glass transition temperature such as the nanoparticles described in or nanoparticles made by the methods described in U.S. Pat. No. 8,518,963, the contents of which are herein incorporated by reference in its entirety. As another non-limiting example, the polymer nanoparticle for oral and parenteral formulations may be made by the methods described in European Patent No. EP2073848B1, the contents of which are herein incorporated by reference in its entirety.

In another embodiment, the recombinant SS18::SSX fusion peptide epitopes and at least one cancer-testis antigen (CTA) epitope, or nucleic acids (e.g., mRNA, cDNA) encoding the same, described herein may be formulated in nanoparticles used in imaging. The nanoparticles may be liposome nanoparticles such as those described in US Patent Publication No US20130129636, herein incorporated by reference in its entirety. As a non-limiting example, the liposome may comprise gadolinium(III)2-{4,7-bis-carboxymethyl-10-[(N,N-distearylamidomethyl-N amido-methyl]-1,4,7,10-tetra-azacyclododec-1-yl}-acetic acid and a neutral, fully saturated phospholipid component (see e.g., US Patent Publication No US20130129636, the contents of which is herein incorporated by reference in its entirety).

In one embodiment, the nanoparticles which may be used in the present disclosure are formed by the methods described in U.S. Patent Application No. US20130130348, the contents of which is herein incorporated by reference in its entirety.

The nanoparticles of the present disclosure may further include nutrients such as, but not limited to, those which deficiencies can lead to health hazards from anemia to neural tube defects (see e.g, the nanoparticles described in International Patent Publication No WO2013072929, the contents of which is herein incorporated by reference in its entirety). As a non-limiting example, the nutrient may be iron in the form of ferrous, ferric salts or elemental iron, iodine, folic acid, vitamins or micronutrients.

In one embodiment, the recombinant SS18::SSX fusion peptide epitopes and at least one cancer-testis antigen (CTA) epitope, or nucleic acids (e.g., mRNA, cDNA) encoding the same, of the present disclosure may be formulated in a swellable nanoparticle. The swellable nanoparticle may be, but is not limited to, those described in U.S. Pat. No. 8,440,231, the contents of which is herein incorporated by reference in its entirety. As a non-limiting embodiment, the swellable nanoparticle may be used for delivery of the recombinant SS18::SSX fusion peptide epitopes and at least one cancer-testis antigen (CTA) epitope, or nucleic acids (e.g., mRNA, cDNA) encoding the same, of the present disclosure to the pulmonary system (see e.g., U.S. Pat. No. 8,440,231, the contents of which is herein incorporated by reference in its entirety).

The recombinant SS18::SSX fusion peptide epitopes and at least one cancer-testis antigen (CTA) epitope, or nucleic acids (e.g., mRNA, cDNA) encoding the same, of the present disclosure may be formulated in polyanhydride nanoparticles such as, but not limited to, those described in U.S. Pat. No. 8,449,916, the contents of which is herein incorporated by reference in its entirety.

The nanoparticles and microparticles of the present disclosure may be geometrically engineered to modulate macrophage and/or the immune response. In one aspect, the geometrically engineered particles may have varied shapes, sizes and/or surface charges in order to incorporated the recombinant SS18::SSX fusion peptide epitopes and at least one cancer-testis antigen (CTA) epitope, or nucleic acids (e.g., mRNA, cDNA) encoding the same, of the present disclosure for targeted delivery such as, but not limited to, pulmonary delivery (see e.g., International Publication No WO2013082111, the contents of which is herein incorporated by reference in its entirety). Other physical features the geometrically engineering particles may have include, but are not limited to, fenestrations, angled arms, asymmetry and surface roughness, charge which can alter the interactions with cells and tissues. As a non-limiting example, nanoparticles of the present disclosure may be made by the methods described in International Publication No WO2013082111, the contents of which is herein incorporated by reference in its entirety.

In one embodiment, the nanoparticles of the present disclosure may be water soluble nanoparticles such as, but not limited to, those described in International Publication No. WO2013090601, the contents of which is herein incorporated by reference in its entirety. The nanoparticles may be inorganic nanoparticles which have a compact and zwitterionic ligand in order to exhibit good water solubility. The nanoparticles may also have small hydrodynamic diameters (HD), stability with respect to time, pH, and salinity and a low level of non-specific protein binding.

In one embodiment the nanoparticles of the present disclosure may be developed by the methods described in US Patent Publication No. US20130172406, the contents of which are herein incorporated by reference in its entirety.

In one embodiment, the nanoparticles of the present disclosure are stealth nanoparticles or target-specific stealth nanoparticles such as, but not limited to, those described in US Patent Publication No. US20130172406; the contents of which is herein incorporated by reference in its entirety. The nanoparticles of the present disclosure may be made by the methods described in US Patent Publication No. US20130172406, the contents of which are herein incorporated by reference in its entirety.

In another embodiment, the stealth or target-specific stealth nanoparticles may comprise a polymeric matrix. The polymeric matrix may comprise two or more polymers such as, but not limited to, polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, polyamines, polyesters, polyanhydrides, polyethers, polyurethanes, polymethacrylates, polyacrylates, polycyanoacrylates or combinations thereof.

In one embodiment, the nanoparticle may be a nanoparticle-nucleic acid hybrid structure having a high density nucleic acid layer. As a non-limiting example, the nanoparticle-nucleic acid hybrid structure may made by the methods described in US Patent Publication No. US20130171646, the contents of which are herein incorporated by reference in its entirety. The nanoparticle may comprise recombinant SS18::SSX fusion peptide epitopes and at least one cancer-testis antigen (CTA) epitope, or nucleic acids (e.g., mRNA, cDNA) encoding the same.

At least one of the nanoparticles of the present disclosure may be embedded in the core a nanostructure or coated with a low density porous 3-D structure or coating which is capable of carrying or associating with at least one payload within or on the surface of the nanostructure. Non-limiting examples of the nanostructures comprising at least one nanoparticle are described in International Patent Publication No. WO2013123523, the contents of which are herein incorporated by reference in its entirety.

### Modes of Administration and Therapeutic Regimen of Vaccine Compositions of the Present Technology

Pharmaceutical compositions are typically formulated to be compatible with its intended route of administration. Administration of recombinant SS18::SSX fusion peptide epitopes and at least one cancer-testis antigen (CTA) epitope, or nucleic acids (e.g., mRNA, cDNA) encoding the same, of the present technology can be achieved using more than one route. Examples of routes of administration include, but are not limited to parenteral (e.g., intravenous, intramuscular, intraperitoneal, intradermal, subcutaneous), intratumoral, intrathecal, intranasal, systemic, transdermal, iontophoretic, intradermal, intraocular, pleural, intranodal, intrapleural, or topical administration. In one embodiment, recombinant SS18::SSX fusion peptide epitopes and at least one cancer-testis antigen (CTA) epitope, or nucleic acids (e.g., mRNA, cDNA) encoding the same, of the present technology are administered directly into the tumor, e.g., by intratumoral injection, where a direct local reaction is desired. In one embodiment, recombinant SS18::SSX fusion peptide epitopes and at least one cancer-testis antigen (CTA) epitope, or nucleic acids (e.g., mRNA, cDNA) encoding the same, of the present technology are administered intramuscularly, optionally wherein the recombinant SS18::SSX fusion peptide epitopes and at least one cancer-testis antigen (CTA) epitope, or nucleic acids (e.g., mRNA, cDNA) encoding the same, are lipid encapsulated. Additionally, administration routes of recombinant SS18::SSX fusion peptide epitopes and at least one cancer-testis antigen (CTA) epitope, or nucleic acids (e.g., mRNA, cDNA) encoding the same, of the present technology can vary, e.g., first administration using an intratumoral injection, and subsequent administration via an intravenous injection, or any combination thereof. A therapeutically effective amount of recombinant SS18::SSX fusion peptide epitopes and at least one cancer-testis antigen (CTA) epitope, or nucleic acids (e.g., mRNA, cDNA) encoding the same, of the present technology injection can be administered for a prescribed period of time and at a prescribed frequency of administration. In certain embodiments, recombinant SS18::SSX fusion peptide epitopes and at least one cancer-testis antigen (CTA) epitope, or nucleic acids (e.g., mRNA, cDNA) encoding the same, of the present technology can be used in conjunction with other therapeutic treatments. In some embodiments, the therapeutically effective amount of recombinant SS18::SSX fusion peptide epitopes and at least one cancer-testis antigen (CTA) epitope, or nucleic acids (e.g., mRNA, cDNA) encoding the same, are administered to a subject without cancer who is at risk of developing synovial sarcoma. In some embodiments, the recombinant SS18::SSX fusion peptide epitopes and at least one cancer-testis antigen (CTA) epitope, or nucleic acids (e.g., mRNA, cDNA) encoding the same, of the present technology can be administered in a neoadjuvant (preoperative) or adjuvant (postoperative) setting for subjects inflicted with bulky primary tumors. It is anticipated that such optimized therapeutic regimen will induce an immune response against the tumor and reduce the tumor burden in a subject before or after primary therapy, such as surgery. Furthermore, recombinant SS18::SSX fusion peptide epitopes and at least one cancer-testis antigen (CTA) epitope, or nucleic acids (e.g., mRNA, cDNA) encoding the same, of the present technology can be administered in conjunction with other therapeutic treatments such as surgery, chemotherapy or radiation.

In certain embodiments, the recombinant SS18::SSX fusion peptide epitopes and at least one cancer-testis antigen (CTA) epitope, or nucleic acids (e.g., mRNA, cDNA) encoding the same, of the present technology is administered at least once weekly or monthly but can be administered more often if needed, such as two times weekly for several weeks, months, years, or even indefinitely as long as benefits persist. More frequent administrations are contemplated if tolerated and if they result in sustained or increased benefits. Benefits of the present methods include but are not limited to the following: prevention of cancer occurrence, reduced risk of cancer occurrence, reduction of the number of cancer cells, reduction of the tumor size, eradication of tumor, inhibition of cancer cell infiltration into peripheral organs, inhibition or stabilization or eradication of metastatic growth, inhibition or stabilization of tumor growth, and stabilization or improvement of quality of life. Furthermore, the benefits may include induction of an immune response against the tumor, activation of effector CD4⁺ T-cells, an increase of effector CD8⁺ T-cells, or reduction of regulatory CD4⁺ cells.

In certain other embodiments, the tumor mass or tumor cells are treated with recombinant SS18::SSX fusion peptide epitopes and at least one cancer-testis antigen (CTA) epitope, or nucleic acids (e.g., mRNA, cDNA) encoding the same, of the present technology in vivo, ex vivo, or in vitro.

### Effective Amount and Dosage of Vaccine Compositions of the Present Technology

A therapeutically effective amount of recombinant SS18::SSX fusion peptide epitopes and at least one cancer-testis antigen (CTA) epitope, or nucleic acids (e.g., mRNA, cDNA) encoding the same, of the present technology can be administered in one or more divided doses for a prescribed period of time and at a prescribed frequency of administration.

For example, as is apparent to those skilled in the art, a therapeutically effective amount of recombinant SS18::SSX fusion peptide epitopes and at least one cancer-testis antigen (CTA) epitope, or nucleic acids (e.g., mRNA, cDNA) encoding the same, of the present technology in accordance with the present disclosure may vary according to factors such as the disease state, age, sex, weight, and general condition of the subject, and the ability of recombinant SS18::SSX fusion peptide epitopes and at least one cancer-testis antigen (CTA) epitope, or nucleic acids (e.g., mRNA, cDNA) encoding the same, of the present technology to elicit a desired immunological response in the particular subject (the subject's response to therapy). In delivering recombinant SS18::SSX fusion peptide epitopes and at least one cancer-testis antigen (CTA) epitope, or nucleic acids (e.g., mRNA, cDNA) encoding the same, of the present technology to a subject, the dosage will also vary depending upon such factors as the general medical condition, previous medical history, disease type and progression, tumor burden, the presence or absence of tumor infiltrating immune cells in the tumor, and the like.

In general, the subject is administered a dosage of recombinant SS18::SSX fusion peptide epitopes and at least one cancer-testis antigen (CTA) epitope, or nucleic acids (e.g., mRNA, cDNA) encoding the same, of the present technology in the range of between about 10 μg/kg and about 400 μg/kg, optionally in a lipid encapsulated form. In some embodiments, a dosage of 25 micrograms of the recombinant SS18::SSX fusion peptide epitopes and at least one cancer-testis antigen (CTA) epitope, or nucleic acids (e.g., mRNA, cDNA) encoding the same, is included in the vaccine administered to the subject. In some embodiments, a dosage of 100 micrograms of the recombinant SS18::SSX fusion peptide epitopes and at least one cancer-testis antigen (CTA) epitope, or nucleic acids (e.g., mRNA, cDNA) encoding the same, is included in the vaccine administered to the subject. In some embodiments, a dosage of 400 micrograms of the recombinant SS18::SSX fusion peptide epitopes and at least one cancer-testis antigen (CTA) epitope, or nucleic acids (e.g., mRNA, cDNA) encoding the same, is included in the vaccine administered to the subject. In some embodiments, the recombinant SS18::SSX fusion peptide epitopes and at least one cancer-testis antigen (CTA) epitope, or nucleic acids (e.g., mRNA, cDNA) encoding the same, accumulates at a 100 fold higher level in the local lymph node in comparison with the distal lymph node.

In some embodiments, it may be advantageous to formulate compositions of the present disclosure in dosage unit form for ease of administration and uniformity of dosage. “Dosage unit form as used herein” refers to physically discrete units suited as unitary dosages for the mammalian subjects to be treated; each unit containing a predetermined quantity of active material calculated to produce the desired therapeutic effect in association with the required pharmaceutically or veterinary acceptable carrier.

### Therapeutic Methods of the Present Technology

In one aspect, the present disclosure provides a method for treating sarcoma (e.g., synovial sarcoma) in a subject in need thereof, the method comprising administering to the subject an effective amount of recombinant SS18::SSX fusion peptide epitopes and at least one cancer-testis antigen (CTA) epitope, or nucleic acids (e.g., mRNA, cDNA) encoding the same, of the present technology. In some embodiments, the treatment comprises one or more of the following: inducing an immune response in the subject against the tumor or enhancing or promoting an ongoing immune response against the tumor in the subject, reducing the size of the tumor, eradicating the tumor, inhibiting growth of the tumor, inhibiting metastatic growth of the tumor, inducing apoptosis of tumor cells, or prolonging survival of the subject. In one aspect, the present disclosure provides a method for preventing sarcoma (e.g., synovial sarcoma) in a subject at risk of developing cancer, the method comprising prophylactically administering to the subject an effective amount of recombinant SS18::SSX fusion peptide epitopes and at least one cancer-testis antigen (CTA) epitope, or nucleic acids (e.g., mRNA, cDNA) encoding the same, of the present technology. In some embodiments, the subject is a human. In some embodiments, the composition of the present technology comprising the recombinant SS18::SSX fusion peptide epitopes and at least one cancer-testis antigen (CTA) epitope, or nucleic acids (e.g., mRNA, cDNA) encoding the same, is administered to the subject by intratumoral or intravenous injection or a simultaneous (i.e., concurrent) or sequential combination of intratumoral and intravenous injection. In some embodiments, the composition of the present technology comprising the recombinant SS18::SSX fusion peptide epitopes and at least one cancer-testis antigen (CTA) epitope, or nucleic acids (e.g., mRNA, cDNA) encoding the same, is encapsulated in a lipid nanoparticle. In any of the preceding embodiments of the methods disclosed herein, the patient has received at least one prior anti-cancer therapy, such as surgery, radiotherapy, or chemotherapy. In some embodiments, the sarcoma is recurrent or metastatic.

In some embodiments, the subject is diagnosed with synovial sarcoma. Additionally or alternatively, in some embodiments, administration of the recombinant SS18::SSX fusion peptide epitopes and at least one cancer-testis antigen (CTA) epitope, or nucleic acids (e.g., mRNA, cDNA) encoding the same will reduce ctDNA plasma levels in the subject. The sarcoma may be soft tissue sarcoma, synovial sarcoma, myxoid-round cell liposarcoma, malignant peripheral nerve sheath tumor, or undifferentiated pleiomorphic sarcoma.

In any and all embodiments of the methods disclosed herein, the patient harbors a loss-of-function in SETD2. In some embodiments, the vaccine and/or the immune checkpoint inhibitor is administered pleurally, topically, parenterally, intramuscularly, intravenously, subcutaneously, intranodally, intratumorally, intrathecally, intrapleurally or intraperitoneally.

### Combination Therapy

In some embodiments, the vaccine compositions disclosed herein may be combined with one or more additional therapies for the prevention or treatment of sarcoma (e.g., synovial sarcoma). Additional therapeutic agents include, but are not limited to, immune checkpoint blocking agents, chemotherapy, and targeted therapy.

In some embodiments, recombinant SS18::SSX fusion peptide epitopes and at least one cancer-testis antigen (CTA) epitope, or nucleic acids (e.g., mRNA, cDNA) encoding the same, of the present technology are combined or separately, sequentially, or simultaneously (i.e., concurrently) administered with one or more additional anti-cancer therapies, such as immune checkpoint blocking agents, chemotherapy, and targeted therapy.

In some embodiments, the one or more immune checkpoint blocking agents may be an anti-PD-1 antibody, an anti-PD-L1 antibody, an anti-PD-L2 antibody, an anti-CTLA-4 antibody, an anti-TIM3 antibody, an anti-4-1BB antibody, an anti-CD73 antibody, an anti-GITR antibody, or an anti-LAG-3 antibody. In some embodiments, the one or more immune checkpoint blocking agents are selected from the group consisting of cemiplimab, tremelimumab, ipilimumab, nivolumab, pidilizumab, lambrolizumab, pembrolizumab, envafolimab, atezolizumab, avelumab, durvalumab, dostarlimab, MPDL3280A, BMS-936559, MEDI-4736, MSB 00107180, inhibitory antibodies against LAG-3 (lymphocyte activation gene 3), TIM3 (T-cell immunoglobulin and mucin-3), B7-H3, B7-H4, TIGIT (T-cell immunoreceptor with Ig and ITIM domains), AMP-224, MDX-1105, arelumab, tremelimumab, IMP321, MGA271, BMS-986016, lirilumab, urelumab, PF-05082566, IPH2101, MEDI-6469, CP-870,893, Mogamulizumab, Varlilumab, Galiximab, AMP-514, AUNP 12, Indoximod, NLG-919, INCB024360, CD80, CD86, ICOS (inducible T-cell costimulatory), DLBCL (diffuse large B-cell lymphoma) inhibitors, BTLA (B and T lymphocyte attenuator), PDR001, and any combination thereof.

Examples of chemotherapeutic agents include, but are not limited to, anthracyclines (e.g., epirubicine, doxorubicin), palifosfamide, evofosfamide, dacarbazine, Eribulin, ifosfamide, and trabectedin. Examples of targeted therapy include olaratumab, cixutumumab, R1507, figitumumab, imatinib, sorafenib, gefitinib, panobinostat, pazopanib and regorafenib.

Dosage ranges of the foregoing are known in or readily within the skill in the art as several dosing clinical trials have been completed, making extrapolation to other agents possible.

In any case, the multiple therapeutic agents may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If not simultaneous, the timing between the multiple doses may vary from more than zero weeks to less than four weeks. In addition, the combination methods, compositions and formulations are not to be limited to the use of only two agents.

### Kits

The present disclosure provides for kits comprising one or more compositions comprising recombinant SS18::SSX fusion peptide epitopes and at least one cancer-testis antigen (CTA) epitope, or nucleic acids (e.g., mRNA, cDNA) encoding the same, of the present technology together with instructions for administration to a subject to be treated (e.g., a subject at risk for synovial sarcoma). The instructions may indicate a dosage regimen for administering the composition or compositions as provided herein. In some embodiments, the recombinant SS18::SSX fusion peptide epitopes and at least one cancer-testis antigen (CTA) epitope, or nucleic acids (e.g., mRNA, cDNA) encoding the same, may be lipid encapsulated. In some embodiments, the kit may further comprise materials for lipid encapsulating the recombinant SS18::SSX fusion peptide epitopes and at least one cancer-testis antigen (CTA) epitope, or nucleic acids (e.g., mRNA, cDNA) encoding the same, and/or instructions for achieving encapsulation.

The present disclosure provides a variety of kits for conveniently and/or effectively carrying out methods of the present disclosure. Typically kits will comprise sufficient amounts and/or numbers of components to allow a user to perform multiple treatments of a subject(s) and/or to perform multiple experiments.

In one aspect, the present disclosure provides kits comprising the recombinant SS18::SSX fusion peptide epitopes and at least one cancer-testis antigen (CTA) epitope, or nucleic acids (e.g., mRNA, cDNA) encoding the same, of the present disclosure.

The kits can be for protein production, comprising a first polynucleotide encoding recombinant SS18::SSX fusion peptide epitopes and at least one cancer-testis antigen (CTA) epitope. The kit may further comprise packaging and instructions and/or a delivery agent to form a formulation composition. The delivery agent may comprise a saline, a buffered solution, a lipidoid or any delivery agent disclosed herein.

In one embodiment, the buffer solution may include sodium chloride, calcium chloride, phosphate and/or EDTA. In another embodiment, the buffer solution may include, but is not limited to, saline, saline with 2 mM calcium, 5% sucrose, 5% sucrose with 2 mM calcium, 5% Mannitol, 5% Mannitol with 2 mM calcium, Ringer's lactate, sodium chloride, sodium chloride with 2 mM calcium and mannose (See e.g., U.S. Pub. No. 20120258046; herein incorporated by reference in its entirety). In a further embodiment, the buffer solutions may be precipitated or it may be lyophilized. The amount of each component may be varied to enable consistent, reproducible higher concentration saline or simple buffer formulations.

The components may also be varied in order to increase the stability of the recombinant SS18::SSX fusion peptide epitopes and at least one cancer-testis antigen (CTA) epitope, or nucleic acids (e.g., mRNA, cDNA) encoding the same, in the buffer solution over a period of time and/or under a variety of conditions.

In one aspect, the present disclosure provides kits for protein production, comprising: a polynucleotide comprising a translatable region encoding the recombinant SS18:SSX fusion peptide epitopes and at least one cancer-testis antigen (CTA) epitope, provided in an amount effective to produce a desired amount of the recombinant SS18::SSX fusion peptide epitopes and at least one cancer-testis antigen (CTA) epitope when introduced into a target cell; a second polynucleotide comprising an inhibitory nucleic acid, provided in an amount effective to substantially inhibit the innate immune response of the cell; and packaging and instructions.

In one aspect, the present disclosure provides kits for protein production, comprising a polynucleotide comprising a translatable region, wherein the polynucleotide exhibits reduced degradation by a cellular nuclease, and packaging and instructions.

In one aspect, the present disclosure provides kits for protein production, comprising a polynucleotide comprising a translatable region, wherein the polynucleotide exhibits reduced degradation by a cellular nuclease, and a mammalian cell suitable for translation of the translatable region of the first nucleic acid.

The kits may optionally include instructions customarily included in commercial packages of therapeutic products, that contain information about, for example, the indications, usage, dosage, manufacture, administration, contraindications and/or warnings concerning the use of such therapeutic products.

### EXAMPLES

Those skilled in the art will recognize that several embodiments are possible within the scope and spirit of this present application. The present application will now be described in greater detail by reference to the following non-limiting examples. The following examples further illustrate the present application but, of course, should not be construed as in any way limiting its scope.

### Example 1: H3K36 Methylation-Dependent Crosstalk and Vulnerabilities in SS

Loss of H3K36 methyltransferase function in SS. Leveraging data from an institutional clinical genomic profiling initiative based on the MSK-IMPACT large panel NGS assay which covers more than 50 epigenetic regulators, we examined the genetic alterations in SS samples from 74 patients. In this cohort, loss-of-function somatic alterations in methyltransferases responsible for mono-, di-, and/or tri-methylating histone H3 lysine 36 (H3K36) were observed in ~10% of cases, predominantly in SETD2.

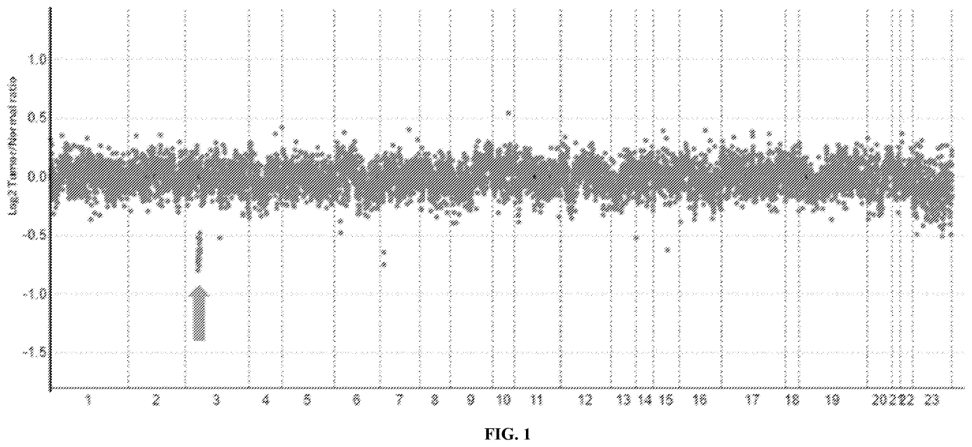

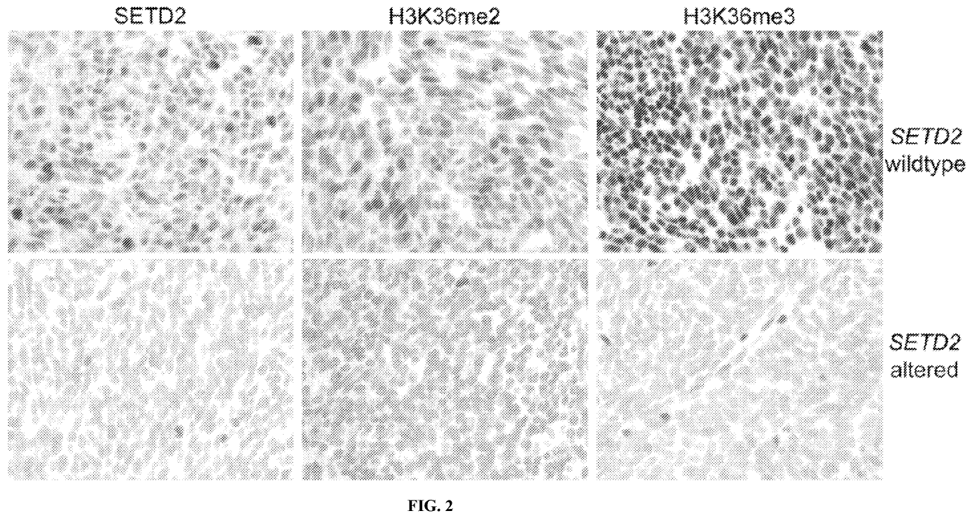

Because SETD2 is the only H3K36 methyltransferase capable of catalyzing trimethylation (H3K36me3), whereas mono- and di-methylation can be deposited by several methyltransferases, we focused on SETD2 alterations. While the proportion of SS with SETD2 alterations may seem small, it is nonetheless remarkable in the setting of the quiet genomes of SS with few other recurrent mutations or copy number changes. The paucity of copy number changes is also exemplified by the case shown in FIG. 1. Notably, SETD2 inactivation is at least as common in SS as TP53 mutations. SS with SETD2-inactivating mutations show loss of H3K36me3 but not H3K36me2 (FIG. 2). Exploratory survival analyses suggest that SETD2 inactivation in SS patients may be associated with worse overall survival, similar to non-overlapping cases with TP533MDM2 alterations. With approximately 5% of SS showing SETD2 inactivation, assuming a median time to event of 4 years in the worse-outcome group, this should provide 83% power to detect a hazard ratio (HR) of 2.8 and 88%, power to detect an HR of 3 at a two-sided significance level of 0.05.

Another, independent line of evidence that points to the importance of H3K36 methylation in SS is the finding that the H3K36me2 demethylase KDM2B (which therefore antagonizes deposition of the H3K36me3 mark) is an essential gene in SS. KDM2B levels are relatively high in human SS cell lines compared to normal fibroblasts or other cancer cell lines. Taken together with the prominent H3K36me3 expression in SETD2 wild type SS, this suggests that SS cells may have a strong activation of H3K36 methylation that is dynamically kept in check with sustained KDM2B expression or permanently abrogated by SETD2 loss.

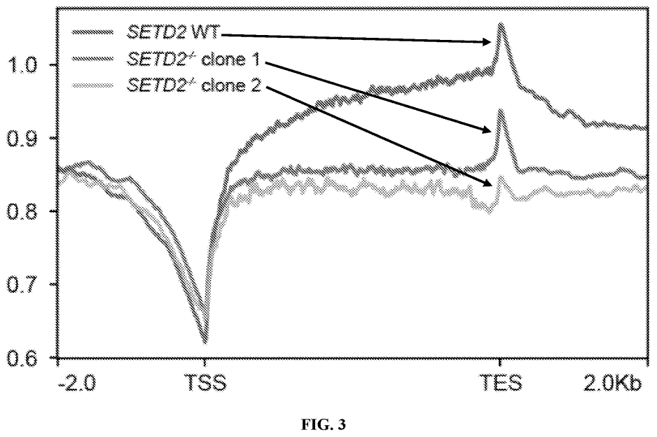

Thus, dysregulation of H3K36 methylation is observed in a subset of SS and may modulate disease behavior. Given a well-established role for epigenetic crosstalk in SS, it was therefore hypothesized that perturbation of H3K36 methylation triggers epigenetic reprogramming that alters SS18::SSX localization and function and induces dependencies on specific epigenetic regulators. To model the frequent SETD2 loss-of-function genetic events in SS patients, we used a CRISPR strategy to generate a SETD2^{-/-} HS-SY-II human SS cell line in which we previously knocked in a FLAG-HA tag at the N-terminus of SS18::SSX, thereby allowing robust interrogation of the effects of the altered H3K36me3 landscape on the genomic localization of the driver oncoprotein. SETD2^{-/-} cells lose H3K36me3 by IHC (FIG. 2) and specifically in actively transcribed genes as determined by chromatin immunoprecipitation followed by DNA sequencing (ChIP-seq) (FIG. 3). In addition to this unique engineered cell line reagent, a bank of patient-derived xenografts from 29 SS patients, including 2 with SETD2 inactivation, were obtained one of which has yielded a SETD2^{-/-} patient-derived cell line (cell line development from the second is in progress). This represents a unique resource, as none of the currently widely available SS cell lines have a SETD2 inactivating event.

Understanding how altered H3K36 methylation modulates SS18::SSX function. The histone mark H3K36me3 is associated with actively transcribed genes, where it recruits DNA methyltransferases, enhances nucleosome deposition following passage of RNA polymerase II (RNAPII), and opposes the deposition of the transcriptionally repressive histone mark, H3K27me3, which is deposited by the polycomb repressive complex 2 (PRC2). Thus, SETD2 mediates epigenetic regulatory activities that are directly relevant to the pathobiology of SS18::SSX, namely polycomb activity and DNA methylation. Given this crosstalk and alterations in H3K36 methyltransferases, including SETD2, in ~10% of SS, it is hypothesized that depletion of H3K36me3 in SETD2^{-/-} SS alters the targeting and function of the SS18::SSX fusion oncoprotein through rewiring of the epigenetic landscape.

To delineate how SETD2 loss of function reprograms the epigenome in SS, we will use isogenic pairs of WT and SETD2^{-/-} human SS cells expressing FLAG-HA-tagged SS18::SSX (see above). The genomic localization of the PRC2-mediated repressive mark, H3K27me3, as well as H3K36me3 and H3K36me2, will be determined using Cleavage Under Targets and Release Using Nuclease (CUT&RUN), which is routine in our laboratory. In addition, we will determine the chromatin occupancy of the FLAG-HA-SS18::SSX fusion, the SS18::SSX-interacting PRC1.1 component KDM2B, and PRC2 (via core subunit SUZ12). For all CUT&RUN experiments, commercially available antibodies that are validated for ChIP-seq, which yields highly concordant results with CUT&RUN assays, will be used. Spike-in E. coli DNA will be used to normalize for library preparation efficiency and reads between samples.

Given the crosstalk between H3K36me3 and DNA methylation, we will next test the hypothesis that SETD2 loss decreases DNA methylation at actively transcribed genes by comparing DNA methylation between WT and SETD2^{-/-} cells using bisulfite sequencing. Finally, we will determine whether chromatin accessibility is altered in SETD2^{-/-} cells versus WT, putatively as a result of impaired H3K36me3 nucleosome deposition following RNAPII passage, using the Assay for Transposase-Accessible Chromatin with sequencing (ATAC-seq).

Having defined the dysregulated epigenetic landscape resulting from SETD2 loss of function, we will determine how these changes alter the targeting and genomic localization of SS18::SSX. We hypothesize that loss of DNA methylation predicted to result from decreased H3K36me3 will recruit SS18::SSX via KDM2B, which binds unmethylated DNA. We will apply CUT&RUN using an anti-HA antibody to track the localization of the epitope-tagged SS18::SSX fusion. We will determine whether this new recruitment pattern depletes SS18::SSX from its canonical binding sites (e.g. homeobox transcription factors), causes it to occupy new loci, or both. We will apply gene ontology analysis to identify biologic categories associated with these occupancy changes, focusing on programs that control differentiation and lineage commitment.

To understand the functional consequences of epigenetic dysregulation in SETD2^{-/-}SS, we will compare gene expression patterns between WT and SETD2^{-/-} isogenic cell lines using RNA-seq. These data will be integrated with the SS18::SSX occupancy data and other epigenomic datasets to test the association between these changes and altered gene expression. To deconvolute SS18::SSX-dependent and -independent SETD2^{-/-}-induced changes, we will employ SS18::SSX shRNA knockdown (compared to a scrambled control). As a control for a possible role of non-histone SETD2 methylation substrates including tubulin, we will introduce the H3K36M histone mutation, which dramatically impairs H3K36me3 deposition⁴⁵, into HS-SY-II cells and compare gene expression patterns with SETD2^{-/-}. All CUT&RUN, ATAC-seq, and RNA-seq studies will be performed in biologic triplicate to ensure the rigor and reproducibility of our conclusions.

Finally, we will validate key conclusions regarding SS18::SSX chromatin occupancy, H3K36me3 and DNA methylation landscapes, and gene expression changes in WT vs. SETD2^{-/-} SS patient tissues (n=15-18 of each genotype) and PDXs (n=2-3 of each), a unique resource.

Expected outcome: We expect to observe decreased H3K36me3, redistributed H3K27me3, decreased DNA methylation, and decreased accessibility of genes related to mesenchymal differentiation, leading to lower expression of these genes. These findings will provide a broad mechanistic model for how epigenetic alterations interact with the SS18::SSX fusion to support tumorigenesis and progression in SS.

Understanding how SETD2 loss alters tumor-intrinsic determinants of immunity in SS. Epigenetic mechanisms are important tumor-intrinsic mediators of immune escape. For instance, transcriptional repression of antigen-presenting machinery, as well as transposable elements (TEs) and downstream IFNγ signaling, are dependent on DNA methylation and the activity of PRC2. This appears to be true in sarcomas based on our preliminary data demonstrating an association between TE depression, increased baseline inflammatory infiltrates, and improved response to immune checkpoint blockade (ICB) on clinical trials. As described above, there is extensive crosstalk between SETD2-dependent H3K36me3, DNA methylation, and PRC2 activity. Consistent with this, expression of the H3K36M histone mutant, which blocks deposition of H3K36me3, triggers de-repression of TEs. In addition, loss of SETD2-promoted nucleosome deposition following passage of RNAPII leads to aberrant expression of normally silenced cryptic transcripts. Loss of epigenetic silencing of cryptic transcripts has immunogenic potential. Therefore, we hypothesize that loss of SETD2 in SS may enhance its antigenicity through several possible mechanisms. To test this, we will analyze the RNA-seq dataset generated in Aim 1a to compare TE expression in WT vs. SETD2^{-/-} SS cell lines. To gain mechanistic insight, regions of TE de-repression will be characterized with respect to SETD2^{-/-}-induced changes in the epigenomic landscape described in the experiments described herein.

We will focus on H3K36me2, which can be globally increased and locally redistributed upon loss of H3K36me3 and is responsible for silencing TEs. Using the same RNA-seq dataset, we will apply gene ontology and gene set enrichment analysis to identify SETD2^{-/-}-induced differences in expression of sensors of double-stranded DNA, antigen-presenting machinery, and type I interferon pathways. Any observed differenced in TE expression or inflammatory programs in these SS cells will be confirmed by RNA-seq of bio-banked fresh frozen WT vs. SETD2^{-/-} patient-derived samples, in which we will also determine differences in immune infiltrates based on expression data (MCP-counter)54 and by multiplex immunofluorescence. Because epigenetic drugs that could potentially induce TE de-repression are FDA-approved (e.g. decitabine, tazemetostat) and therefore could be repurposed into SS clinical trials, we will determine if these drugs synergize with SETD2 loss to increase TE de-repression and tumor-intrinsic immunity. If so, this observation could inform the design of a future biomarker (SETD^{-/-})-driven basket trial evaluating a combination of one of these agents with ICB.

To study the effect of SETD2 loss on the expression of cryptic transcripts, we will again analyze the RNA-seq dataset discussed above. We will computationally determine if these transcripts harbor novel peptides predicted to be presented by MHC-I for immune recognition. In a similar approach, we will analyze the derepressed TEs as sources of neoantigens.

Expected outcome: These results will define how SETD2 loss affects tumor immunogenicity in SS. We expect to observe increased expression of antigen-presenting machinery, TEs, cryptic transcripts encoding neoantigens, and/or IFNγ signaling.

Identifying epigenetic dependencies in SETD2^{-/-} and other types of SS using small molecule probes. Epigenetic pathways are interdependent and SETD2 is involved in crosstalk with multiple epigenetic regulators as described above. Therefore, we hypothesize that disruption of this tightly regulated epigenetic landscape by SETD2 loss in SS creates targetable induced dependencies on other epigenetic regulators. To test this, we will utilize chemical tools now available for manipulating epigenetic regulators. Specifically, we will screen a collection of 800 epigenetically targeted small molecules. The effects of these compounds at 5 μM concentration on viability at 96 h in an ATPase-based assay will be determined in 384-well format. We will compare the potency of drugs between WT vs. SETD2^{-/-} cell lines as well as control IMR and BJ fibroblasts. Doxorubicin, used in the clinical treatment of SS, will be used as a positive control and vehicle (DMSO) as a negative control for normalization. To increase rigor, experiments will be repeated in triplicate and the normalized viability ratio will be used to generate a t-score for each compound. We will rank the 20 most selective compounds based on t-score difference for each of two comparisons (SETD2^{-/-} vs. WT in the epitope-tagged isogenic SS18::SSX background and SETD2-WT SS cell lines vs. fibroblasts). The SETD2-WT vs. fibroblast comparison will allow exclusion of compounds non-specific for SETD2 loss, but that could also warrant further investigation as active epigenetic compounds in a broad SS context in future work.

The IC₅₀s for these top compounds will be determined by generating dose-response curves in all cell lines. The top 5 most promising candidates will then be identified based on the following criteria: 1) selectivity over control cell lines (SETD2^{-/-} vs. WT and fibroblasts), 2) more advanced clinical development status, 3) favorable pharmacokinetics based on published data, 4) a well validated single target. The antitumor activity of selected compounds will then be assessed vs. vehicle control in WT and SETD2^{-/-} PDX models. The putative target will be validated in cell lines using an inducible CRISPR knockout, shRNA, and chemically distinct compounds with the same target, if available, where impaired viability upon target inactivation using all 3 approaches (with none in controls) would demonstrate target selectivity. To determine each compound's epigenetic mechanism of action, we will compare their effects on the epigenetic landscape using CUT&RUN, DNA methylation profiling, or ATAC-seq (depending on the molecule's target) between the cell line and corresponding control in which activity was initially noted (SETD2^{-/-} vs. WT and fibroblast).

### Example 2: Immunogenicity of an SS18::SSX Public Neoantigen and the Antitumor Efficacy of T Cells Genetically Engineered with an SS18::SSX Public Neoantigen T Cell Receptor

Soft tissue sarcomas (STSs) typically harbor many fewer non-synonymous somatic mutations (NSSMs) relative to epithelial malignancies. Because a subset of NSSMs generate T cell-activating neoantigens, this finding could explain why most STSs fail to respond to current immunotherapies. However, many STSs are associated with driver fusions, in which the sequence of one gene is fused to another. Driver fusions might represent a particularly immunogenic subset of neoantigens because they create novel peptide sequences that diverge significantly from self-proteins. Targeting fusion-derived neoantigens could serve as an innovative new strategy to bring the curative potential of cancer immunotherapy to STSs.

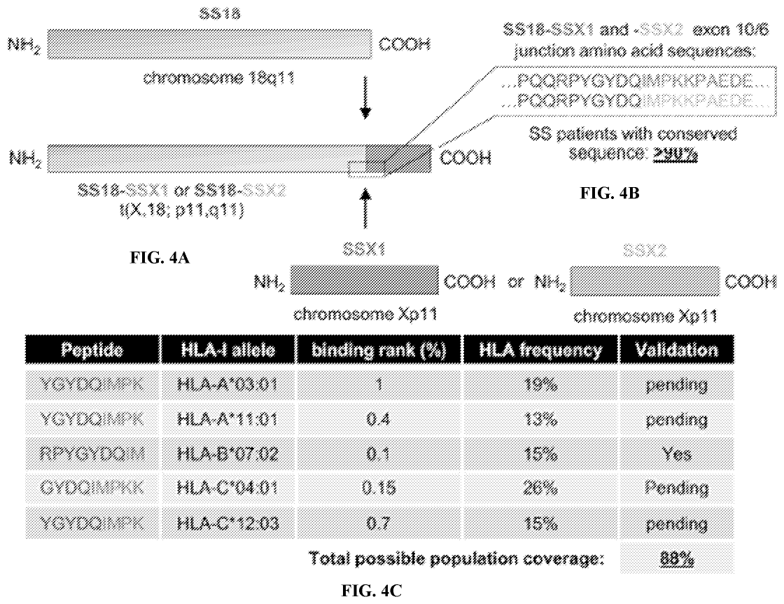

Synovial sarcoma (SS) is a prototypical fusion-driven sarcoma characterized by pathognomonic t(X;18)(p11.2;q11.2) chromosomal translocation that generates an in-frame fusion of SS18 with one of three SSX genes. In >90% of cases, the translocation breakpoint involves the intronic region between SS18 exons 10/11 and exons 5/6 of the highly homologous SSX1 or SSX2 genes (FIG. 4A). Critically, the amino acid sequences of SSX1 and SSX2 immediately surrounding the breakpoint site are identical (FIG. 4B). Thus, the junctional region of the chimeric SS18::SSX (1/2) protein is uniquely expressed by SS cells and shared by the majority of patients. In silico peptide/human leukocyte antigen (HLA) binding algorithms predict a series of junction spanning high-affinity binders to prevalent HLA class I (HLA-I) alleles (FIG. 4C). Collectively, these alleles are expressed by ~88% of North Americans. Notably, one of the predicted junction-spanning peptide sequences has previously been confirmed to be naturally presented and capable of stimulating a fusion-specific T cell response. Based on these findings, it was hypothesized that shared (or ‘public’) neoantigens derived from the SS18::SSX(1/2) proteins are immunogenic and targetable using a TCR gene therapy approach.

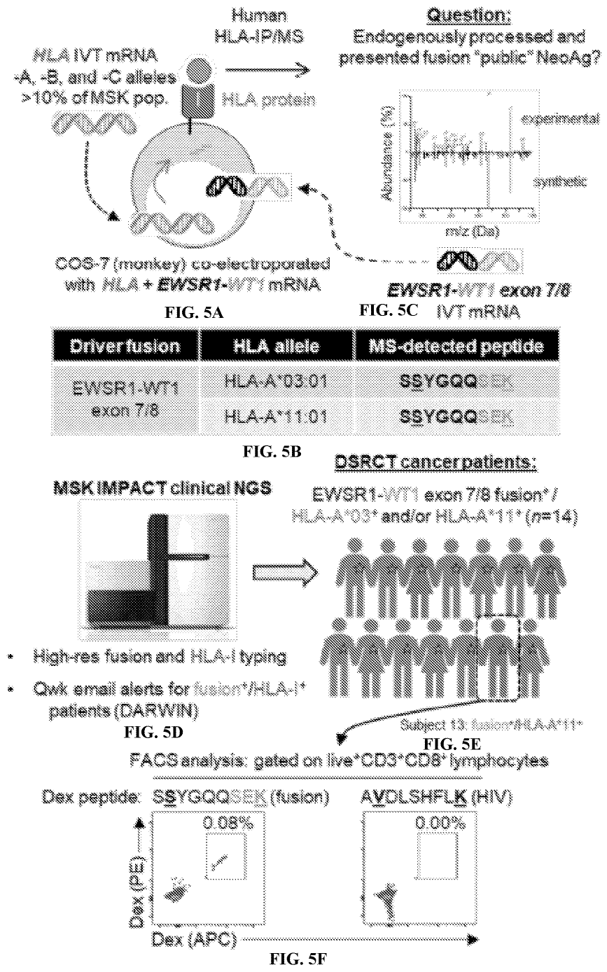

We recently developed an immuno-peptidomic screen to identify naturally processed peptides resulting from mutated driver oncogenes that are presented by prevalent HLA-I alleles. The method works as follows: COS-7 is co-electroporated with in vitro transcribed (IVT) mRNA encoding a full-length mutated driver of interest alongside individual mRNAs encoding HLA-A, -8, and -C alleles expressed by 10% of North Americans (FIG. 5A). COS-7 is a monkey-derived cell line that degrades proteins via proteasomal processing similar to human cells but lacks human HLA expression. Next, human HLA complexes are immune-precipitated from electroporated COS-7, HLA-bound peptides are eluted, and human peptide sequences are identified by mass spectrometry (HLA-IP/MS). The sequence of identified peptides is confirmed by comparing screen-derived MS spectra with spectra resulting from synthetic peptides of known identity. Finally, to establish whether physiologic expression of these proteins is sufficient for neoantigen formation, confirmatory HLA-IP/MS is performed on a cancer cell line or POX that expresses the mutated gene and HLA-I allele of interest.

In preliminary experiments, we applied this method to test whether a recurrent oncogenic gene fusion (EWSR1-WT1 exons 7/8 fusion) expressed by a STS (DSRCT) generates a fusion-derived public neoantigen (FIG. 5A). Using this approach, we discovered that a junction-spanning peptide (sequence: SSYGQQSEK (SEQ ID NO: 11)) resulting from the EWSR1-WT1 fusion protein is processed and presented in the context of HLA-A*03 and HLA-A*11 (FIGS. 5B-5C). Together, these two HLA-I alleles are expressed by ~⅓ of North Americans. HLA-A*03 and HLA-A*11 share 98.6% sequence homology⁶⁹ and frequently bind similar peptides. We subsequently established that physiologic levels of the fusion and HLA-I allele are sufficient to generate the neoantigen by performing HLA-IP/MS on BOD, an HLA-A*03+ DSRCT cell line harboring the EWSR1-WT1 exon 7/8 fusion.

Next, we tested whether MS-identified fusion neoantigens are immunogenic. We prospectively collected peripheral blood mononuclear cell (PBMC) samples from fusion⁺/HLA-I⁺ DSRCT patients under an IRS-approved protocol (#17-250) (FIGS. 5D-5E). As MSK-IMPACT clinical next-generation sequencing (NGS) platform can report both the fusion status and HLA-I haplotype of a patient's tumor for research purposes, we used DARWIN, an automated genotype-driven enrollment tool that matches NGS results with upcoming clinic visits, to flag fusion⁺/HLA-I⁺ DSRCT patients for biospecimen collection. In preliminary experiments, we measured by FACS the frequency of circulating T cells specific for the fusion neoantigen using PBMC samples from an HLA-A*03⁺ and -A*11⁺ DSRCT patient. To quantify antigen-specific T cells, we used fluorochrome-conjugated dextramers composed of synthetic HLA-A*03 and HLA-A*11 molecules loaded with the HLA-IP/MS identified fusion neoantigen peptides. As specificity controls, samples were stained with HLA-A*03 or -A*11 dextramers loaded with an irrelevant HIV-associated peptide. In both patients, we detected CD8⁺ T cells specific for the fusion-derived neoantigen (FIG. 5F and data not shown). In contrast, no T cells stained with the control dextramers. Together, these data demonstrate that our HLA-IP/MS screen can identify immunogenic fusion-derived neoantigens that drive the clonal expansion of antigen-specific CD8⁺ T cells in patients with a STS.

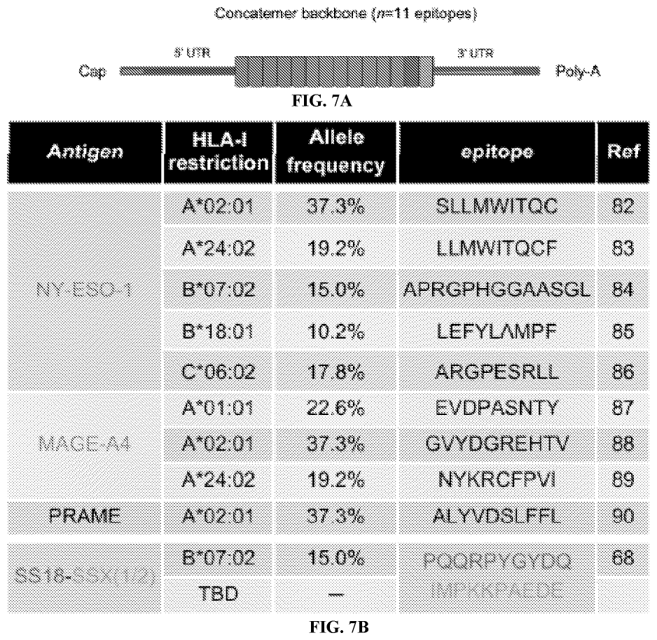

Defining the HLA-restricted immunopeptidome resulting from endogenous processing and presentation of the SS18:: SSX fusion protein. We hypothesized that the recurrent SS18::SSX1 and —SSX2 fusion proteins will generate shared neoantigens restricted by prevalent HLA-I alleles (FIG. 7). To test this hypothesis, we will perform an initial screen by co-electroporating COS-7 with in vitro transcribed (IVT) mRNA encoding the full-length SS18::SSX1 exon 10/6 fusion protein and mRNAs encoding individual HLA-A, —B, and —C alleles present in 10% of North Americans. As a positive control, COS-7 will be electroporated with IVT mRNA encoding HLA-B*07:02. An SS18::SSX1 fusion-derived peptide was previously shown to be presented in the context of HLA-B*07:02 and capable of stimulating antigen-specific T cells with specificity for an HLA-B*07:02+SS cell line. All screen “hits” will be replicated using the identified HLA-I allele and IVT mRNA encoding full-length SS18::SSX2. This will allow us to determine whether minor differences in the C-terminal sequences of SSX1 and SSX2 influence the proteasomal degradation of the fusion proteins. Identified peptide sequences will be validated by comparing the MS spectra of screen hits with that of synthetic peptides.

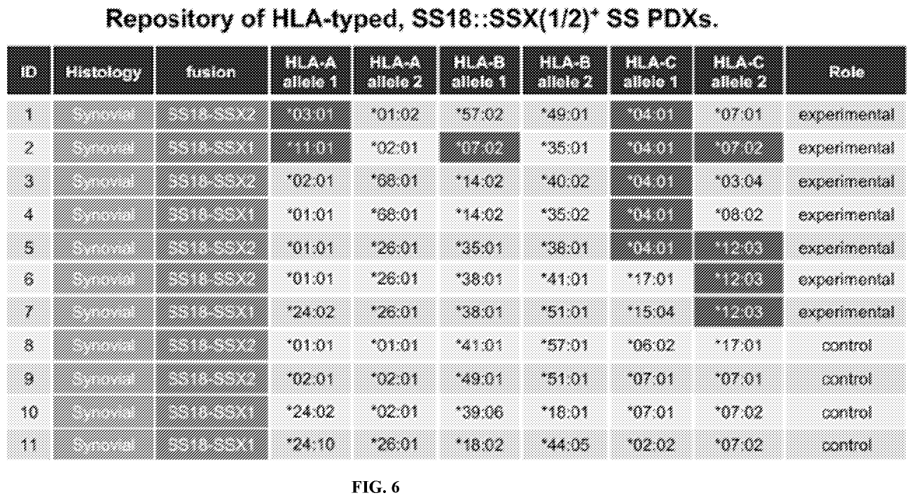

Once we have empirically determined that a junction-spanning peptide sequence is capable of presentation by a prevalent HLA-I allele, we will next establish whether physiologic expression levels of HLA-I and the SS18::SSX(1/2) fusion proteins are sufficient to generate a presented neopeptide. To accomplish this goal, we will use our unique collection of n=11 molecularly-typed SS PDXs of defined HLA-I and SS18::SSX fusion status (FIG. 6). In these experiments, PDXs containing at least one screen-identified HLA-I allele will be grown to ~1 cm² in NSG mice, excised, and made into a single-cell suspension without enzymatic digestion to preserve surface HLA expression. Tumor cells will then be immediately lysed for HLA-IP/MS.

Expected outcomes: We expect to identify ≥1 HLA-I restricted fusion neoantigen in the MS screen in addition to the previously described HLA-B*07:02-restricted peptide.

Retrieving TCR α/β gene sequences associated with clonally expanded SS patient-derived T cell populations that recognize an epitope derived from the SS18::SSX(J 2) junction sequence. We hypothesize that SS patients whose tumors express the SS18::SSX(1/2) exon 10/6 fusion will develop a T cell response to an HLA-IP/MS-identified fusion public neoantigen. To test this hypothesis, we will prospectively establish a biobank of PBMC samples from fusion⁺/HLA-I⁺ SS patients using the MSK-IMPACT NGS platform (FIG. 5D). By studying T cell responses to fusion-derived public neoantigens in a cohort of SS patients, we will be able to quantitatively measure the biologic variables that determine HLA-I restricted neoantigen immunogenicity and immune escape.

Given the prevalence of the HLA-I alleles predicted to bind an SS18::SSX(1/2) fusion neopeptide (FIG. 4C), we anticipate collecting PBMC samples from n≥6 unique fusion+ SS subjects for each HLA-I allele of interest over a 12-24 month period. We will screen by FACS for T cell responses to the fusion-derived neoantigen using dual-color dextramer staining. Samples containing a frequency of dextramer⁺ CD8⁺ T cells≥0.2% will be labeled with a DNA bar-coded version of the dextramer and submitted directly for 10× single-cell V(D)J plus feature barcode sequencing to retrieve paired α/β TCR gene sequences. Samples containing between 0.02%-0.19% of dextramer⁺ T cells will first undergo in vitro stimulation (IVS) to expand the frequency of fusion neoantigen-specific T cells prior to performing single-cell sequencing. This will ensure that a sufficient number of antigen-specific T cells are sampled to identify paired TCR gene sequences with high confidence. IVS will be performed by pulsing T cells with 1 μM of the HLA-IP/MS-identified fusion peptide(s) in media supplemented with the T cell homeostatic cytokines IL-21, IL-7, and IL-15. Samples containing <0.02% dextramer+ T cells will be considered non-reactive. TCR sequences associated with the DNA barcode will be cloned into γ-retroviral expression vectors (RV) for functional validation. TCR function will be assessed using two independent assays: 1) the capacity to bind fluorochrome-labeled dextramers loaded with the fusion peptide but not a control peptide, and 2) the capacity to release TNFα when cocultured with HLA-I⁺COS-7 target cells pulsed with 1 μM of the MS-identified fusion peptide but not a control peptide.

Expected outcomes: These results will help identify HLA haplotypes most likely to respond to the SS18::SSX mRNA component. Based on preliminary data, it is anticipated that PBMC from ≥1 patient with each HLA-I allele identified in our HLA-IP/MS screen will have a detectable public neoantigen specific T cell response. We expect that polyclonal CD8⁺ T cells retrovirally transduced with a retrieved TCR gene sequence will confer fusion neoantigen-specific HLA multimer binding and cytokine release to the fusion peptide but not a control peptide.

Validating reactivity of cloned fusion neoantigen TCR gene sequences to SS18::SSX fusion neoantigen⁺ PDXs in vitro and tumor regression in vivo in a preclinical model. We hypothesize that T cells genetically engineered with a functionally validated fusion neoantigen-reactive TCR will mediate HLA-dependent tumor cytolysis in vitro and regression of an established POX in vivo in an immune-deficient animal model. To test this hypothesis, we will use our panel of fusion neoantigen expressing SS PDXs (FIG. 6). Measurements of antigen-specific CD8⁺ T cell killing will be made using an xCELLigence impedance based real-time cytolytic assay. All experiments will contain a fusion⁺POX line that does not express a correct HLA-I allele (henceforth, HLA-I⁻) and CD8⁺ T cells transduced with an irrelevant influenza-specific TCR as controls for fusion neoantigen expression and TCR specificity, respectively. Assays will be performed with n=3 biologic replicates per condition and repeated ≥2× in cohorts of n=10 age-matched female NSG mice/condition. Mice will be implanted subcutaneously with 5×10⁶ POX cells (FIG. 6; HLA-I⁺ PDX=experimental; HLA-I⁻=controls) in 1:1 PBS/Matrigel. Once tumors are ~50-100 mm³ in volume, mice will be randomized to receive a single infusion of 5×10⁶ CD8⁺ TcR⁺ cells (SS18::SSX(1/2) fusion public neoantigen TCR=experimental; viral-specific TCR=control) by tail-vein injection or left untreated as controls. All mice will receive IL-15 (1 μg/dose) i.p. every 2 days to facilitate T cell engraftment. Tumor volume and animal survival will be measured by an investigator blinded to treatment groups.

Expected outcomes: We expect T cells transduced with a fusion neoantigen-specific TCR will mediate specific cytolysis of HLA-I⁺/fusion⁺ PDXs in vitro and delayed tumor growth and/or tumor regression in vivo but not impact HLA-I-fusion⁺ controls. We expect that T cells modified with a control TCR will have no impact.

### Example 3: Tolerability, Immunogenicity, and Antitumor Efficacy of a Multivalent mRNA Vaccine Encoding the SS18-SSX(1/2) Junction Sequence and Multiple CTAs Highly Expressed in SS Patients

SS typically expresses high levels of multiple CTAs, including NY-ESO-1, MAGE-A4, and PRAME. It is hypothesized that an “off-the-shelf” mRNA vaccine (SSmRNA vaccine) encoding the SS18::SSX(1/2) fusion sequence and multiple CTAs will be immunogenic in SS patients and capable of inducing objective antitumor responses.

We have previously shown that targeting CTAs using T cell therapy leads to promising efficacy for selected patients with SS and myxoid-round cell liposarcoma. These data further highlight the antitumor potential of immunologically targeting multiple members of the CTA family. In addition, the junctional region of the SS18::SSX (1/2) protein is expressed in nearly all patients with SS. Peptide/HLA binding algorithms predict a series of junction-spanning high-affinity binders to multiple prevalent HLA-I alleles (FIG. 4C). At least one of these peptide sequences has been shown to stimulate cytolytic T cell responses against SS cells in vitro.

This is a phase I/II, single-arm, open-label study of the safety, immunogenicity, and antitumor efficacy of SSmRNA vaccine with or without the anti-PD-L1 antibody envafolimab in patients with recurrent or metastatic synovial sarcoma. SSmRNA is composed of a lipid nanoparticle formulation encoding selected sequences of the cancer-germline antigens NY-ESO-1, MAGE-A4, PRAME proteins in addition to the SS18::SSX(1/2) junction amino acid sequence (FIG. 7A). Eligible patients will have metastatic or recurrent synovial sarcoma with RECIST evaluable disease at baseline and have at least one HLA-I allele previously established to bind a peptide contained in the vaccine cassette (FIG. 7B). Patients must be at least 18 years of age and have adequate hematological and end-organ function. Patients with clinically relevant autoimmune disease will be excluded. Patients will remain on study until disease progression or the patient withdraws consent. During the phase II portion of the clinical trial, patients will undergo baseline and on-treatment tumor biopsies.

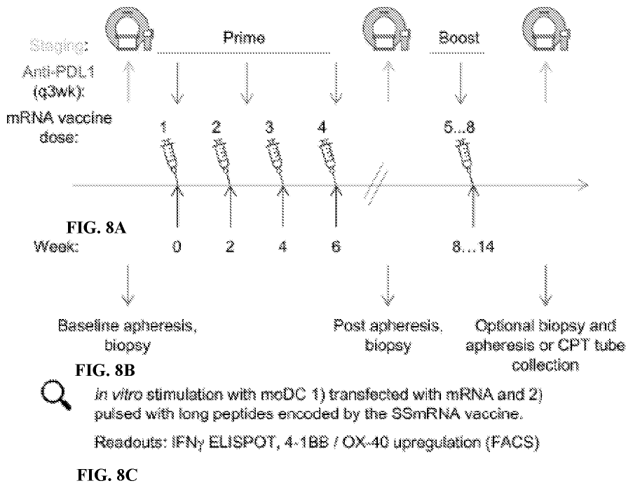

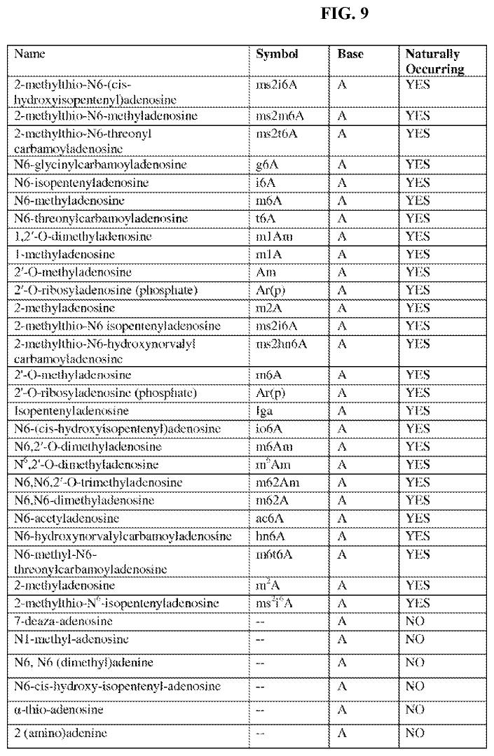

Establishing the safety, immunogenicity, and antitumor activity of an mRNA vaccine encoding an epitope “cassette” (SSmRNA) in combination with PD-L1 blockade in SS patients. Phase Ia. Safety and tolerability of the recommended phase 2 dose (RP2D) of SSmRNA (0.39 mg of total RNA) will be assessed during cycle 1. Participants will receive up to 8 doses of SSmRNA every other week (FIG. 8) via intramuscular injection. If 2 or more of the 6 patients at this dose level experience a dose-limiting toxicity, then a dose de-escalation schema will be introduced. Phase Ib. After the RP2D is confirmed, the combination therapy safety lead-in (n=6 patients) will the evaluate the RP2D of SSmRNA with a fixed dose of 300 mg envafolimab based on safety in a trial in melanoma.

Phase II. The primary outcome of phase II is objective/overall response rate (ORR), defined as the proportion of patients whose tumors display a complete or partial response after initiation of treatment as determined according to the Response Evaluation Criteria in Solid Tumors (RECIST v1.1). Secondary outcomes include clinical benefit rate, median duration of response, progression-free survival (PFS), and overall survival (OS), and will be calculated for all evaluable patients among the 30 enrolled. Exploratory objectives include determination of the preexisting variance and changes to the immune microenvironment that correlate with the clinical efficacy of combined SSmRNA and envafolimab. The immune microenvironment will be characterized by multiplex TIC (CD3, CD8, CD4, FOXP3 and PD-L1) and bulk RNAseq. RNAseq will employ the nCounter® RNA: Protein, PanCancer Immune Profiling Panel, which includes 770 immune-related genes. Expression of CTAs (by IHC through CF1) will be assessed at baseline.

Statistical considerations: Phase Ia. The number of participants enrolled will be dependent on the Dose-limiting toxicity (DLT) incidence. The estimated number of participants, a minimum of 2 evaluable and up to 6 (if no dose de-escalation), should provide adequate information for the safety objectives of the study. AEs will be summarized by preferred term, system organ class, NCI-CTCAE v5.0 grade of severity, and relationship to SSmRNA or envafolimab.

Phase Ib. Patients in this phase (n=6) will initiate treatment at the RP2D dose of SSmRNA in combination with envafolimab 300 mg SC q3 weeks. If there are more than 2 dose-limiting toxicities identified within the first cycle of treatment, this dose regimen will be deemed unsafe and a dose de-escalation schema will be introduced. The schema for de-escalation will depend on the type of DLT.

Phase I. The phase II portion of the study will use a one-stage design based on the exact binomial test. A 10% response rate will be considered not promising; a 30% response rate will be considered promising. This design has a type I error rate of 0.07 and a type II error rate of 0.08. The study will be called positive if there are 6 or more responses out of the 30 patients. Based on the exact binomial test, this study design provides a 92% probability of a positive result if the true response rate is at least 30% and a 93% probability of a negative result if the true response rate is 10% or lower. AEs of the combination will be summarized as described above. PFS and OS rates at 6 and 12 months will be calculated, along with median PFS and OS after ≥12 months of follow-up for all patients.

Measuring the T cell response to polypeptides encoded by the SSmRNA vaccine before and longitudinally after immunization. We hypothesize that SSmRNA vaccination will induce the priming and clonal expansion of CD8⁺ and CD4⁺ T cells specific for epitopes derived from the vaccine. We will test this hypothesis by comparing the frequency of circulating CD4⁺ and CD8⁺ T cells reactive to vaccine-encoded epitopes in pre- and post-priming (within 2 weeks following the 4^{th} dose) and post-boosting (after the 8^{th} dose, in patients with either stable disease or an unconfirmed objective response after priming) blood specimens using two complementary functional immunologic assays (ELISPOT and 4-1BB/OX-40 upregulation). Monocyte-derived dendritic cells (moDCs) will be generated from each patient using material from the baseline apheresis product. Autologous moDCs will either be electroporated with in vitro transcribed (IVT) mRNA encoding individual CTAs, IVT mRNA encoding the junction sequence of the SS18::SSX(1/2) fusion proteins, or pulsed with overlapping long peptides from these same antigens. Electroporation and peptide pulsing will ensure that epitopes are loaded into the endogenous and exogenous antigen presentation pathways, maximizing the likelihood of detecting both HLA-I and HLA class II (HLA-11)-restricted T cell responses. T cells will then be co-cultured at a ratio of 4:1 with moDCs for ~18 h in cytokine-free culture media. T cells cultured alone or co-cultured with moDCs that were not peptide-pulsed or electroporated will serve as negative controls. T cells cultured in the presence of PMA/ionomycin will serve as a positive control. After filters are collected for IFNγ ELISPOT, cells will be analyzed by FACS using fluorochrome-conjugated antibodies specific for CD3, CD4, CD8, 4-1BB (CD137), and OX-40 (CD134) in addition to a viability stain. 4-1BB and OX-40 are costimulatory markers in the TNF superfamily that have very low expression on resting T cells but are acutely upregulated following TCR ligation. All co-cultures will be performed in biologic triplicates. A positive response will be defined as: ELISPOT: ≥50 spots in the experimental group and >2.5× the number of spots present in the negative controls; 4-1BB and/or OX-40: ≥0.2% of gated CD8⁺ or CD4⁺ T cells and >2.5× the frequency of cells in the negative controls.

Resolving the TCR clonotypes and HLA restriction for T cells responding to the CTA and/or SS18::SSX fusion sequences. We hypothesize that clonally expanded TCR gene sequences will confer specific reactivity to vaccine-encoded CTAs and/or the SS18:SSX(1/2) junctional amino acid sequence. To test this hypothesis, we will FACS-sort T cells obtained from the circulation of patients who received the SSmRNA vaccine that upregulate 4-1BB and/or OX-40 following co-culture with autologous moDCs. Individual T cells will be sorted into a 96-well plate containing RT-PCR buffer, and TCR gene sequences will be obtained by 2 nested PCR reactions using multiple Vα and Vβ region primers and 1 primer for Cα and Cβ regions using a one-step RT-PCR kit. For the second amplification, an aliquot of the first RT-PCR product will be used as a template using HotStarTaq DNA polymerase and multiple internally nested Vα and Vβ region primers and 1 internally nested primer for Cα and Cβ regions. The PCR products will be purified and Sanger-sequenced with internally nested Cα and Cβ region primers. Retrieved TCR gene sequences will be cloned into retroviral expression vectors in which the TRA V-J encoding-sequences are fused to the mouse TCRβ constant chain, and TRB V-D-J-encoding sequences are fused to mouse TCRα constant chain. Murinization of the TCR constant chains will serve as a transduction marker and help facilitate proper TCR chain pairing.

To functionally validate retrieved TCR sequences, non-specific CD8⁺ and CD4⁺ T cells will be individually transduced with each retrieved TCR candidate, then co-cultured with autologous moDCs electroporated with the same IVT mRNA or long peptides used to isolate the T cell population. Controls will include T cells co-cultured with mock-electroporated moDCs or moDCs electroporated with an HIV-specific epitope (negative) or in the presence of PMA/ionomycin (positive). A functional TCR will be defined as a TCR that triggers upregulation of 4-1BB and/or OX-40 when transduced into non-specific T cells and co-cultured with moDCs expressing the cognate antigen but not negative controls. To infer whether the TCR is HLA-I or HLA-II restricted, T cells will be co-cultured in the presence of either an HLA-A/B/C blocking antibody (clone: W6/32), an HLA-DR/DP/DQ blocking antibody (clone: IVA12) or treated with a non-specific IgG control antibody. Based on these results, an aliquot of the patient's PBMC will undergo high-resolution HLA-I or HLA-II typing. COS-7 cells will then be co-electroporated with IVT mRNA encoding an individual HLA-I or HLA-II allele from the patient together with IVT mRNA encoding the target antigen. Transfected COS-7 cells will be plated overnight in a 96-well round-bottom plate in complete media to allow surface p/HLA-I or p/HLA-II complex expression, TCR-transduced T cells will be added in at an E:T ratio of 1:1, and 4-1BB/OX-40 upregulation will be measured by FACS as described above.

Expected outcomes. Given the activity already demonstrated with T cell therapy targeting CTAs, the immunogenicity of the SS18::SSX fusion and the mRNA vaccination approach, we expect that SSmRNA with envafolimab will be safe, tolerable, and immunogenic and provide a new therapeutic option for patients with fusion-driven sarcomas and other malignancies that are generally resistant to checkpoint inhibitors.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

1. A vaccine comprising a polypeptide comprising at least one SS18::SSX fusion junction epitope and an oligomer of at least one cancer-testis antigen (CTA) epitope, wherein the at least one SS18::SSX fusion junction sequence comprises the amino acid sequence of PQQRPYGYDQIMPKKPAEDE (SEQ ID NO: 1), optionally wherein the at least one SS18::SSX fusion junction epitope is operably linked to the oligomer.

2. A vaccine comprising a nucleic acid molecule encoding a polypeptide comprising at least one SS18::SSX fusion junction epitope and an oligomer of at least one cancer-testis antigen (CTA) epitope, wherein the at least one SS18::SSX fusion junction sequence comprises the amino acid sequence of PQQRPYGYDQIMPKKPAEDE (SEQ ID NO: 1), optionally wherein the at least one SS18::SSX fusion junction epitope is operably linked to the oligomer.

3. The vaccine of claim 1, wherein the at least one SS18::SSX fusion junction epitope is located at the N-terminus or the C-terminus of the oligomer.

4. The vaccine of claim 1, wherein the at least one SS18::SSX fusion junction epitope comprises exon 10 or exon 11 of SS18 and exon 5 or exon 11 of SSX1 or SSX2.

5. The vaccine of claim 1, wherein
the at least one CTA epitope is derived from one or more of NY-ESO-1, PRAME, or MAGEA4; or
the at least one CTA epitope comprises a NY-ESO-1 epitope comprising one or more amino acid sequences selected from among SLLMWITQC (SEQ ID NO: 2), LLMWITQCF (SEQ ID NO: 3), APRGPHGGAASGL (SEQ ID NO: 4), LEFYLAMPF (SEQ ID NO: 5) and ARGPESRLL (SEQ ID NO: 6); or
the at least one CTA epitope comprises a MAGE-A4 epitope comprising one or more amino acid sequences selected from among EVDPASNTY (SEQ ID NO: 7), GVYDGREHTV (SEQ ID NO: 8), and NYKRCFPVI (SEQ ID NO: 9); or
the at least one CTA epitope comprises a PRAME epitope comprising the amino acid sequence of ALYVDSLFFL (SEQ ID NO: 10); or
the oligomer is a dimer, a trimer, a tetramer, a pentamer, a hexamer, a heptamer, an octamer, a nonamer, a decamer, an 11-mer, a 12-mer, a 13-mer, a 14-mer, a 15-mer, a 16-mer, a 17-mer, an 18-mer, a 19-mer, a 20-mer, a 21-mer, a 22-mer, a 23-mer, a 24-mer, a 25-mer, a 26-mer, a 27-mer, a 28-mer, a 29-mer, or a 30-mer; or
the oligomer of the at least one CTA epitope comprises tandem repeats of a single CTA epitope, optionally wherein the tandem repeats of the single CTA epitope are linked via a linker; or
the oligomer of the at least one CTA epitope comprises at least 2 distinct CTA epitopes, optionally wherein the at least 2 distinct CTA epitopes are linked via a linker; or
the linker is a cleavable peptide linker or a rigid peptide linker; or
the oligomer of the at least one CTA epitope further comprises one or more sequences of any one of SEQ ID NOs: 2-10; or
the polypeptide further comprises a leader peptide at its N-terminus and an MHC class I trafficking signal (MITD) at its C terminus; or
the vaccine further comprises a pharmaceutically acceptable carrier selected from the group consisting of a cream, emulsion, gel, liposome, nanoparticle, or ointment; or
the vaccine is formulated within a nanoparticle, wherein the nanoparticle has a mean diameter of 50-200 nm; or
the SS18::SSX fusion junction epitope and/or the at least one CTA epitope are presented by one or more of HLA-A*02:01, HLA-A*03:01, HLA-A*11:01, HLA-B*07:02, HLA-C*04:01, HLA-C*12:03, HLA-A *01:01 HLA-A *24:02 HLA-A *24:10 HLA-A *01:02 HLA-A *68:01, HLA-A *26:01 HLA-B*57:02, HLA-B*14:02, HLA-B* 35:01, HLA-B*38:01, HLA-B*41:01, HLA-B*49:01, HLA-B*39:06, HLA-B* 18:02, HLA-B*40:02, HLA-B*35:02, HLA-B*51:01, HLA-B*57:01, HLA-B*18:01, HLA-B*44:05, HLA-C*17:01, HLA-C*15:04, HLA-C*06:02, HLA-C*07:01, HLA-C* 02:02, HLA-C*03:04, HLA-C*07:02, HLA-C*08:02, or HLA-C*07:02.

6. (canceled)

7. (canceled)

8. (canceled)

9. (canceled)

10. (canceled)

11. (canceled)

12. (canceled)

13. The vaccine of claim 5, wherein the linker comprises a GS4 linker (SEQ ID NO: 13), a GS3 linker (SEQ ID NO: 14), a P2A linker, a T2A linker, an E2A linker, a F2A linker, a BmCPV2A linker, an AAY linker, a GPGPG linker (SEQ ID NO: 15), an EAAAK linker (SEQ ID NO: 16), a HEYGAEALERAG linker (SEQ ID NO: 17), a KK linker, or an RVRR linker (SEQ ID NO: 18).

14. (canceled)

15. (canceled)

16. The vaccine of claim 2, wherein the nucleic acid molecule comprises RNA or DNA.

17. The vaccine of claim 2, wherein the nucleic acid molecule comprises RNA, and wherein each uracil residue in the RNA comprises a chemical modification and/or at least one 5′ terminal cap.

18. The vaccine of claim 17, wherein the chemical modification is in the 5-position of each uracil residue.

19. The vaccine of claim 17, wherein the chemical modification is selected from the group consisting of pseudouridine, N1-methylpseudouridine, 2-thiouridine, 4′-thiouridine, 5-methylcytosine, 2-thio-1-methyl-1-deaza-pseudouridine, 2-thio-l-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine, and 2′-O-methyl uridine.

20. The vaccine of claim 17, wherein the at least one 5′ terminal cap is a naturally occurring cap, a synthetic cap or an optimized cap.

21. (canceled)

22. (canceled)

23. The vaccine of claim 5, wherein the nanoparticle is a lipid nanoparticle.

24. The vaccine of claim 23, wherein the nanoparticle has a polydiversity value of less than 0.4 or a net neutral charge at a neutral pH.

25. The vaccine of claim 5, wherein the nanoparticle is a cationic lipid nanoparticle comprising a cationic lipid, a PEG-modified lipid, a sterol and a non-cationic lipid.

26. The vaccine of claim 25, wherein the cationic lipid is selected from the group consisting of 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319).

27. The vaccine of claim 25, wherein the cationic lipid nanoparticle has a molar ratio of about 20-60% cationic lipid, about 5-25% non-cationic lipid, about 25-55% sterol; and about 0.5-15% PEG-modified lipid.

28. (canceled)

29. A method for preventing or treating sarcoma in a patient in need thereof comprising administering to the patient an effective amount of the vaccine of claim 1.

30. The method of claim 29, wherein
the sarcoma is recurrent or metastatic; or
the patient has received at least one prior anti-cancer therapy, optionally wherein the at least one prior anti-cancer therapy is surgery, radiotherapy, or chemotherapy; or
the method further comprises sequentially, simultaneously or separately administering to the subject an effective amount of an immune checkpoint inhibitor; or
the patient harbors a loss-of-function in SETD2; or
the vaccine is administered pleurally, topically, parenterally, intramuscularly, intravenously, subcutaneously, intranodally, intratumorally, intrathecally, intrapleurally or intraperitoneally; or
the sarcoma is soft tissue sarcoma, synovial sarcoma, myxoid-round cell liposarcoma, malignant peripheral nerve sheath tumor, or undifferentiated pleiomorphic sarcoma.

31. (canceled)

32. (canceled)

33. The method of claim 30, wherein the immune checkpoint inhibitor comprises one or more of an anti-PD-1 antibody, an anti-PD-L1 antibody, an anti-PD-L2 antibody, an anti-CTLA-4 antibody, an anti-TIM3 antibody, an anti-4-1BB antibody, an anti-CD73 antibody, an anti-GITR antibody, and an anti-LAG-3 antibody; or wherein the immune checkpoint inhibitor is administered pleurally, topically, parenterally, intramuscularly, intravenously, subcutaneously, intranodally, intratumorally, intrathecally, intrapleurally or intraperitoneally.

34. The method of claim 33, wherein
the anti-PD-L1 antibody comprises envafolimab, atezolizumab, avelumab, or durvalumab; or
wherein the anti-PD-1 antibody comprises pembrolizumab, nivolumab, or Cemiplimab; or
wherein the CTLA-4 antibody comprises ipilimumab or tremelimumab.

35. (canceled)

36. (canceled)

37. (canceled)

38. (canceled)

39. (canceled)

40. (canceled)
